(19) United States
(12) Patent Application Publication
(10) Pub. No.: US 2025/0025575 A1
(43) Pub. Date: Jan. 23, 2025

(54) Compositions and Methods for Cell-Specific Expression of Target Genes

(71) Applicant: THE TRUSTEES OF PRINCETON UNIVERSITY, Princeton, NJ (US)

(72) Inventors: Carola Maturana, Princeton, NJ (US); Esteban Engel, Princeton, NJ (US)

(21) Appl. No.: 18/712,552

(22) PCT Filed: Nov. 23, 2022
(86) PCT No.: PCT/US2022/080438

§ 371 Date: May 22, 2024

Related U.S. Application Data
(60) Provisional application No. 63/264,464, filed on Nov. 23, 2021.

(51) Int. Cl. A61K 48/00; C12N 15/86

(52) U.S. Cl. CPC A61K 48/0058; A61K 48/0075; C12N 15/86; C12N 2750/14143; C12N 2830/008

(57) ABSTRACT

Provided are polynucleotides comprising a transcriptional control element comprising a promoter sequence having at least 80% sequence identity to SEQ ID NO: 1. SEQ ID NO:2. SEQ ID NO:3 or SEQ ID NO:4 and a cell- or tissue-specific enhancer element. Also provided are compositions (e.g., vectors, viruses, host cells, and pharmaceutical compositions) comprising said polynucleotides, methods that are useful for expressing a target gene in a host cell, and methods that are useful for expressing a target gene in a subject in need thereof.

### RELATED APPLICATION(S)

This application claims the benefit of U.S. Provisional Application No. 63/264,464, filed on Nov. 23, 2021. The entire teachings of the above application are incorporated herein by reference.

### GOVERNMENT SUPPORT

This invention was made with government support under Grant Nos. OD010996 and TR003017 awarded by the National Institutes of Health. The government has certain rights in the invention.

### INCORPORATION BY REFERENCE OF MATERIAL IN XML

This application incorporates by reference the Sequence Listing contained in the following extensible Markup Language (XML) file being submitted concurrently herewith:
a) File name: 53911034001.xml; created Nov. 23, 2022, 534,323 Bytes in size.

### BACKGROUND

Viruses infect their hosts and introduce their genetic material into cells of the host as part of their replication cycle. This genetic material contains basic “instructions” for producing more copies of these viruses by hijacking the body's normal production machinery to serve the needs of the virus. The host cell will carry out these instructions and produce additional copies of the virus, leading to more and more cells of the host becoming infected. As such, viruses can be used as vehicles to carry genes that may provide therapeutic benefits into a cell.

### SUMMARY

The disclosure generally relates to polynucleotides (e.g., vectors), viruses (e.g., AAV), host cells, and pharmaceutical compositions, methods that are useful for expressing a target gene in a host cell, and methods that are useful for expressing a target gene in a subject in need thereof.

In one aspect, the present disclosure provides a polynucleotide, wherein the isolated polynucleotide comprises a transcriptional control element comprising a promoter sequence having at least 80% sequence identity to SEQ ID NO:1, SEQ ID NO:2, SEQ ID NO: 3 or SEQ ID NO:4 and a cell- or tissue-specific enhancer element.

In some embodiments, the enhancer element comprises a nucleotide sequence identical to any one of SEQ ID NOs: 5-176 or SEQ ID NO: 350. In certain embodiments, the transcriptional control element comprises a nucleotide sequence that is identical to any one of SEQ ID NOs: 177-349.

In some embodiments, the polynucleotide further comprises a coding sequence for a target gene operably linked to the transcriptional control element. In particular embodiments, the coding sequence for the target gene comprises at least 4 kilobases.

In some embodiments, the polynucleotide is a vector. In certain embodiments, the vector is a gene therapy vector. In certain embodiments, the vector is a viral vector. In particular embodiments, the viral vector is an adeno-associated virus (AAV) vector.

In another aspect, the present disclosure provides a virus comprising one or more of the isolated polynucleotides described herein. In some embodiments, the virus is an AAV.

In another aspect, the present disclosure provides a host cell comprising one or more of the isolated polynucleotides or viruses described herein. In some embodiments, the host cell is a mammalian cell. In certain embodiments, the host cell is a myoblast, a pancreatic cell, a lung cell, a neonatal retina cell, a microglia cell, an astrocyte or a neuron.

In another aspect, the present disclosure provides a pharmaceutical composition comprising one or more of the isolated polynucleotides, viruses or host cells described herein, and one or more pharmaceutically acceptable excipients, diluents, or carriers.

In another aspect, the present disclosure provides a method of expressing a target gene in a host cell, wherein the method comprises contacting the host cell with any one of the polynucleotides or viruses described herein under conditions whereby the polynucleotide or the virus is introduced into the host cell, and expression of the target gene occurs in the host cell.

In another aspect, the present disclosure provides a method of expressing a target gene in a subject in need thereof, wherein the method comprises administering to the subject an effective amount of any one or more of the polynucleotides, viruses or pharmaceutical compositions described herein, whereby expression of the target gene occurs in a host cell of the subject.

### BRIEF DESCRIPTION OF THE DRAWINGS

The foregoing will be apparent from the following more particular description of example embodiments, as illustrated in the accompanying drawings in which like reference characters refer to the same parts throughout the different views. The drawings are not necessarily to scale, emphasis instead being placed upon illustrating embodiments.

FIG. 1 depicts the complete nucleotide sequence of PRV LAP of 902 base pairs (bp), and the sub-regions LAP1 of 498 bp (bold and underlined), LAP2 of 404 bp (underlined), and LAP1_2 of 880 bp. LAP1_2 includes most of the LAP1 and LAP2 sequences, but lacks the first 22 nucleotides of LAP1. The boxes without numbers depict consensus sequences for transcription factors (TFs) including, the GC box: specificity protein 1 and 3 (Sp1 and Sp3); the CCAAT box: nuclear factor Y (NF-Y); and the TATA box: TATA-binding protein (TBP). Numbered boxes indicate the coordinates for the binding motif sites of the TFs: 1: SRY-Box 10 (SOX10); 2: CAMP response element-binding protein (CREB); 3: CCCTC-binding factor (CTCF); 4: oligodendrocyte transcription factor 2 (Olig2); 5: signal transducer and activator of transcription (STAT1).

FIGS. 2A-2D show images of primary superior cervical ganglia (SCG) neurons transduced with AAV containing novel LAP2 variants. AAV-PHP.eB-driven mCherry expression in SCG neurons is shown at 5 days post infection with AAV containing LAP2 full length (SEQ ID NO:4) (FIG. 2A); EnhancerLAP2.169 (SEQ ID NO:345) (FIG. 2B); EnhancerLAP2.170 (SEQ ID NO:346) (FIG. 2C), or EnhancerLAP2.171 (SEQ ID NO:347) (FIG. 2D). SCG neurons were transduced with 3×10¹¹ AAV vg/dish. Scale bar, 50 μm.

FIGS. 3A-3C show MiniLAP2 drives transgene expression in mouse brain as efficiently as LAP2 following systemic administration. FIG. 3A shows the MiniLAP2 sequence. FIG. 3B shows representative immunofluorescence images of nuclei staining in Sham-negative control (top), LAP2 (middle), and MiniLAP2 (bottom). FIG. 3C shows representative immunofluorescence images of anti-mCherry staining for Sham-negative control (top), LAP2 (middle), and MiniLAP2 (bottom). Scale bar, 1 mm.

FIGS. 4A-4O show representative confocal images showing anti-mCherry signal for AAV-PHP.eB-MiniLAP2 in (FIG. 4A) anterior olfactory nucleus, (FIG. 4B) cortex, somatosensory areas, (FIG. 4C) cortex, visual areas, (FIG. 4D) striatum, (FIG. 4E) pallidum, (FIG. 4F) hippocampus CA1, (FIG. 4G) hippocampus CA2, (FIG. 4H) hippocampus CA3, (FIG. 4I) hippocampus dentate gyrus, (FIG. 4J) hypothalamus, (FIG. 4K) midbrain, superior colliculus, (FIG. 4L) midbrain, inferior colliculus, (FIG. 4M) hindbrain, pontine reticular, (FIG. 4N) medulla, spinal nucleus, and (FIG. 4O) cerebellum VI. All images are stacked confocal sections. Scale bar, 100 μm.

FIG. 4P shows the quantification of the indirect fluorescence intensity of anti-mCherry signal driven by AAV-LAP2 and AAV-MiniLAP2 at 30 days post injection (dpi) is shown in cortex, hippocampus, striatum, and cerebellum.

FIG. 4Q shows the quantification of percentage of anti-mCherry signal positive of total cells driven by AAV-LAP2 and AAV-MiniLAP2 at 30 days post injection (dpi) is shown in cortex, hippocampus, striatum, and cerebellum. Data are represented as mean±SEM; N=3 (three tissue sections were analyzed for each animal). Significance was determined with Student's t test. A p value<0.05 was statically significant (NS, non-significant).

FIGS. 5A1-5A4 show representative confocal images of sagittal cortex brain sections from mice, showing immunostaining with anti-mCherry antibody (top) and a neuronal marker (bottom). FIG. 5A1 shows expression in neurons. FIG. 5A2 shows expression in astrocytes. FIG. 5A3 shows expression in microglia. FIG. 5A4 shows expression in oligodendrocytes. Scale bar, 100 μm.

FIGS. 5B1-5B4 show representative confocal images of sagittal hippocampus brain sections from mice, showing immunostaining with anti-mCherry antibody (top) and neurons labeled with the pan-neuronal marker NeuN (bottom). FIG. 5B1 shows expression in neurons. FIG. 5B2 shows expression in astrocytes. FIG. 5B3 shows expression in microglia. FIG. 5B4 shows expression in oligodendrocytes. Scale bar, 100 μm.

FIGS. 6A-E show representative confocal images of sagittal brain sections from mice, showing immunostaining with anti-mCherry antibody (top) and a neuronal marker (bottom). FIG. 6A shows expression in the hippocampus CA1. FIG. 6B shows expression in the cerebellum VIII. FIG. 6C shows expression in the substantia nigra. FIG. 6D is a representative confocal image of cross lumbar spinal cord. FIG. 6E is a higher magnification image of the dorsal horn. Scale bar, 100 μm and 200 μm.

FIG. 6F shows quantification of anti-mCherry signal positive of total cells driven by AAV-LAP2 (73%) and AAV-MiniLAP2 (2%) at 30 dpi. A p value<0.05 was statically significant (*** p<0.001; NS, non-significant). Two-way ANOVA with Turkey's post hoc test.

FIGS. 7A-7C show the localization of PHP.eB-LAP2 and PHP.eB-MiniLAP2 in kidney (FIG. 7A), skeletal muscle (FIG. 7B), and liver (FIG. 7C).

FIG. 7D shows representative images for mCherry immunostaining and corresponding morphometric analysis used to quantify mCherry staining from PHP.eB-LAP2 in liver.

FIG. 7E shows representative images for mCherry immunostaining and corresponding morphometric analysis used to quantify mCherry staining from PHP.eB-MiniLAP2 in liver. Higher magnification image of the liver lobule area analyzed. Scale bar, 1 mm, 200 μm and 100 μm.

FIG. 7F shows quantification of anti-mCherry positive nuclei per area analyzed. N=3 (two tissue sections were analyzed per animal).

### DETAILED DESCRIPTION

A description of example embodiments follows.

Several aspects of the disclosure are described below, with reference to examples for illustrative purposes only. It should be understood that numerous specific details, relationships, and methods are set forth to provide a full understanding of the disclosure. One having ordinary skill in the relevant art, however, will readily recognize that the disclosure can be practiced without one or more of the specific details or practiced with other methods, protocols, reagents, cell lines and animals. The present disclosure is not limited by the illustrated ordering of acts or events, as some acts may occur in different orders and/or concurrently with other acts or events. Furthermore, not all illustrated acts, steps or events are required to implement a methodology in accordance with the present disclosure. Many of the techniques and procedures described, or referenced herein, are well understood and commonly employed using conventional methodology by those skilled in the art.

### Polynucleotides of the Disclosure

In one aspect, the present disclosure provides a polynucleotide (e.g., an isolated polynucleotide), wherein the polynucleotide comprises a transcriptional control element comprising a promoter sequence having at least 80% sequence identity to SEQ ID NO:1, SEQ ID NO:2, SEQ ID NO:3 or SEQ ID NO:4 and a cell- or tissue-specific enhancer element.

In some embodiments, the polynucleotide comprises deoxyribonucleotides. In certain embodiments, the polynucleotide comprises ribonucleotides. Non-limiting examples of polynucleotides include single-, double- or multi-stranded DNA or RNA, DNA-RNA hybrids, or a polymer comprising purine and pyrimidine bases, or other natural, chemically, or biochemically modified, non-natural, or derivatized nucleotide bases. The backbone of the polynucleotide can comprise sugars and phosphate groups, modified or substituted sugar or phosphate groups, a polymer of synthetic subunits such as phosphoramidates, or a combination thereof.

In some embodiments, the polynucleotide is an isolated polynucleotide. An “isolated polynucleotide” refers to a polynucleotide that has been separated from other cellular components normally associated with native DNA, including proteins and other DNA sequences.

Polynucleotides can be produced recombinantly or synthetically, using methods, techniques and reagents that are well known in the art, such as routine and well-known molecular cloning techniques and solid-phase synthesis techniques. In some embodiments, a polynucleotide of the disclosure is a recombinant polynucleotide.

### Promoter Sequences

In some embodiments, the promoter sequence in a polynucleotide of the disclosure comprises a nucleotide sequence having at least 50%, at least 55%, at least 60%, at least 65%, at least 70%, at least 75%, at least 80%, at least 85%, at least 90%, at least 95%, at least 96%, at least 97%, at least 98%, or at least 99% sequence identity to a nucleic acid sequence selected from SEQ ID NO:1, SEQ ID NO:2, SEQ ID NO:3 and SEQ ID NO:4.

(SEQ ID NO: 1)
ATATATCTTTTTTTATTTTGTCTGGGCCTGGAGACCCGCAGCAGGAGCG
GAGGTGGGTGCGGGGCCGGGAGCCGGAGCAGGACCGGGAACAGGAACAG
GAACAGGAACAGGAACAGGAACAGGAGTGGGGCCGGGAGCAGGAGCAGG
AGCGGGAGCCGAAGTGGGGGCAGGAGCGGCGGCGGCCGCAGCAGCAACA
GGGTCGCCCCAGTCCGCGGCGAGGAAGAGGGAGCTC
(SEQ ID NO: 2)
CACCCATCACAGCAGCCGCGGACGCTGCGCGCCGGAGCGGTCCATCTC
GCCAGCCAGCCAACCAGCCGAGCCGCCCAGCCGACCCGAGAGCCCCGAG
AGCCAGACTCCCTCAGCCATAGAAGACACCGGGCGGGAGAGACGGACTG
AAAAAATATATCTTTTTTTATTTTGTCTGGGCCTGGAGACCCGCAGCAG
GAGCGGAGGTGGGTGCGGGGCCGGGAGCCGGAGCAGGACCGGGAACAGG
AACAGGAACAGGAACAGGAACAGGAACAGGAGTGGGGCCGGGAGCAGGA
GCAGGAGCGGGAGCCGAAGTGGGGGCAGGAGCGGCGGCGGCCGCAGCAG
CAACA
(SEQ ID NO: 3)
CACCCATCACAGCAGCCGCGGACGCTGCGCGCCGGAGCGGTCCATCTC
GCCAGCCAGCCAACCAGCCGAGCCGCCCAGCCGACCCGAGAGCCCCGAG
AGCCAGACTCCCTCAGCCATAGAAGACACCGGGCGGGAGAGACGGACTG
AAAAAATATATCTTTTTTTATTTTGTCTGGGCCTGGAGACCCGCAGCAG
GAGCGGAGGTGGGTGCGGGGCCGGGAGCCGGAGCAGGACCGGGAACAGG
AACAGGAACAGGAACAGGAACAGGAACAGGAGTGGGGCCGGGAGCAGGA
GCAGGAGCGGGAGCCGAAGTGGGGGCAGGAGCGGCGGCGGCCGCAGCAG
CAACAGGGTCGCCCCAGTCCGCGGCGAGGAAGAGGGAGCTC
(SEQ ID NO: 4)
ATCCCCGGTCCGCGCTCCGCCCACCCATCACAGCAGCCGCGGACGCTG
CGCGCCGGAGCGGTCCATCTCGCCAGCCAGCCAACCAGCCGAGCCGCCC
AGCCGACCCGAGAGCCCCGAGAGCCAGACTCCCTCAGCCATAGAAGACA
CCGGGCGGGAGAGACGGACTGAAAAAATATATCTTTTTTTATTTTGTCT
GGGCCTGGAGACCCGCAGCAGGAGCGGAGGTGGGTGCGGGGCCGGGAGC
CGGAGCAGGACCGGGAACAGGAACAGGAACAGGAACAGGAACAGGAACA
GGAGTGGGGCCGGGAGCAGGAGCAGGAGCGGGAGCCGAAGTGGGGGCAG
GAGCGGCGGCGGCCGCAGCAGCAACAGGGTCGCCCCAGTCCGCGGCGAG
GAAGAGGGAGCTC

As used herein, the term “sequence identity,” refers to the extent to which two nucleotide sequences have the same residues at the same positions when the sequences are aligned to achieve a maximal level of identity, expressed as a percentage. For sequence alignment and comparison, typically one sequence is designated as a reference sequence, to which a test sequences are compared. The sequence identity between reference and test sequences is expressed as the percentage of positions across the entire length of the reference sequence where the reference and test sequences share the same nucleotide or amino acid upon alignment of the reference and test sequences to achieve a maximal level of identity. As an example, two sequences are considered to have 70% sequence identity when, upon alignment to achieve a maximal level of identity, the test sequence has the same nucleotide residue at 70% of the same positions over the entire length of the reference sequence.

Alignment of sequences for comparison to achieve maximal levels of identity can be readily performed by a person of ordinary skill in the art using an appropriate alignment method or algorithm. In some instances, the alignment can include introduced gaps to provide for the maximal level of identity. Examples include the local homology algorithm of Smith & Waterman, Adv. Appl. Math. 2:482 (1981), the homology alignment algorithm of Needleman & Wunsch, J. Mol. Biol. 48:443 (1970), the search for similarity method of Pearson & Lipman, Proc. Nat'l. Acad. Sci. USA 85:2444 (1988), computerized implementations of these algorithms (GAP, BESTFIT, FASTA, and TFASTA in the Wisconsin Genetics Software Package, Genetics Computer Group, 575 Science Dr., Madison, Wis.), and visual inspection (see generally Ausubel et al., Current Protocols in Molecular Biology).

In some embodiments, the promoter sequence comprises a nucleotide sequence selected from SEQ ID NO:1, SEQ ID NO:2, SEQ ID NO:3 and SEQ ID NO:4.

In some embodiments, the promoter sequence comprises a nucleotide sequence having at least 50%, at least 55%, at least 60%, at least 65%, at least 70%, at least 75%, at least 80%, at least 85%, at least 90%, at least 95%, at least 96%, at least 97%, at least 98%, or at least 99% sequence identity to a nucleic acid sequence selected from SEQ ID NO:1. In some embodiments, the promoter sequence comprises a nucleotide sequence selected from SEQ ID NO:1.

In some embodiments, the promoter sequence comprises a nucleotide sequence having at least 50%, at least 55%, at least 60%, at least 65%, at least 70%, at least 75%, at least 80%, at least 85%, at least 90%, at least 95%, at least 96%, at least 97%, at least 98%, or at least 99% sequence identity to a nucleic acid sequence selected from SEQ ID NO:2. In some embodiments, the promoter sequence comprises a nucleotide sequence selected from SEQ ID NO:2.

In some embodiments, the promoter sequence comprises a nucleotide sequence having at least 50%, at least 55%, at least 60%, at least 65%, at least 70%, at least 75%, at least 80%, at least 85%, at least 90%, at least 95%, at least 96%, at least 97%, at least 98%, or at least 99% sequence identity to a nucleic acid sequence selected from SEQ ID NO:3. In some embodiments, the promoter sequence comprises a nucleotide sequence selected from SEQ ID NO:3.

In some embodiments, the promoter sequence comprises a nucleotide sequence having at least 50%, at least 55%, at least 60%, at least 65%, at least 70%, at least 75%, at least 80%, at least 85%, at least 90%, at least 95%, at least 96%, at least 97%, at least 98%, or at least 99% sequence identity to a nucleic acid sequence selected from SEQ ID NO:4. In some embodiments, the promoter sequence comprises a nucleotide sequence selected from SEQ ID NO:4.

### Enhancer Elements

As used herein, “enhancer element” or “enhancer sequence” refers to a sequence of nucleotides that is present in a polynucleotide (e.g., a vector) at a site that is sufficiently close to a gene transcription unit to enhance production of mRNA, and is operative in either position, upstream or downstream, and in either orientation with respect to the gene transcription unit. In some embodiments, the enhancer element or sequence is a sequent that is not present in a LAP2 promoter sequence disclosed herein.

In some embodiments, the isolated polynucleotide comprises an enhancer element that is upstream of the transcription unit. In some embodiments, the isolated polynucleotide comprises an enhancer element that is downstream of the transcription unit. In some embodiments, the isolated polynucleotide comprises an enhancer element that is downstream of the transcription unit and an enhancer element that is downstream of the transcription unit.

In some embodiments, the enhancer element has a length of about 300 nucleotides or less, for example, about: 280, 275, 260, 250, 240, 225, 220, 200, 180, 175, 160, 150, 140, 125, 120, 100, 80, 75, 60 or 50 nucleotides or less. In some embodiments, the enhancer element has a length of about 50 nucleotides to about 300 nucleotides, for example, about: 50-275, 60-300, 60-280, 75-275, 80-280, 75-250, 80-260, 80-240, 100-240, 100-250, 100-225, 100-220, 120-220, 120-200, 125-225, 125-200, 140-200, 140-180, 150-200, 150-175 or 160-180 nucleotides.

In some embodiments, the enhancer element comprises a nucleotide sequence having at least about 70% sequence identity to any one of SEQ ID NOs: 5-176 or SEQ ID NO: 350, for example, having at least about: 75%, 80%, 85%, 90%, 95%, 98%, 99% or 100% sequence identify to any one of SEQ ID NOs: 5-176 or SEQ ID NO: 350. In certain embodiments, the enhancer element comprises a nucleotide sequence that is identical to any one of SEQ ID NOs: 5-176 or SEQ ID NO: 350.

In some embodiments, the enhancer element comprises a nucleotide sequence having at least about 85% sequence identity to any one of SEQ ID NOs: 5-22, SEQ ID NOs: 23-43, SEQ ID NOs: 44-52, SEQ ID NOs: 53-90, SEQ ID NOs: 91-108, SEQ ID NOs: 109-113, SEQ ID NOs: 114-126, SEQ ID NOs: 127-160, SEQ ID NO: 161 or SEQ ID NO: 162, SEQ ID NO: 163 or SEQ ID NO: 164, SEQ ID NOs: 165-169, SEQ ID NO: 170, SEQ ID NO: 171 or SEQ ID NO: 172, or SEQ ID NOs: 173-176, or SEQ ID NO: 350, for example, having at least about: 90%, 91%, 92%, 93%, 94%, 95%, 96%, 97%, 98%, 99% or 100% sequence identity to any one of SEQ ID NOs: 5-22, SEQ ID NOs: 23-43, SEQ ID NOs: 44-52, SEQ ID NOs: 53-90, SEQ ID NOs: 91-108, SEQ ID NOs: 109-113, SEQ ID NOs: 114-126, SEQ ID NOs: 127-160, SEQ ID NO: 161 or SEQ ID NO: 162, SEQ ID NO: 163 or SEQ ID NO: 164, SEQ ID NOs: 165-169, SEQ ID NO: 170, SEQ ID NO: 171 or SEQ ID NO: 172, or SEQ ID NOs: 173-176, or SEQ ID NO: 350.

In certain embodiments, the transcriptional control element comprises a nucleotide sequence that is identical to any one of SEQ ID NOs: 5-22, SEQ ID NOs: 23-43, SEQ ID NOs: 44-52, SEQ ID NOs: 53-90, SEQ ID NOs: 91-108, SEQ ID NOs: 109-113, SEQ ID NOs: 114-126, SEQ ID NOs: 127-160, SEQ ID NO: 161 or SEQ ID NO: 162, SEQ ID NO: 163 or SEQ ID NO: 164, SEQ ID NOs: 165-169, SEQ ID NO: 170, SEQ ID NO: 171 or SEQ ID NO: 172, or SEQ ID NOs: 173-176, or SEQ ID NO: 350.

In some embodiments, the enhancer element is gastrocnemius muscle myoblast-specific. In certain embodiments, the enhancer element comprises a nucleotide sequence having at least about 85% sequence identity to any one of SEQ ID NOs: 5-22, for example, having at least about: 90%, 91%, 92%, 93%, 94%, 95%, 96%, 97%, 98%, 99% or 100% sequence identity to any one of SEQ ID NOs: 5-22. In particular embodiments, the enhancer element comprises a nucleotide sequence that is identical to any one of SEQ ID NOs: 5-22.

In some embodiments, the enhancer element is pancreas cell-specific. In certain embodiments, the enhancer element comprises a nucleotide sequence having at least about 85% sequence identity to any one of SEQ ID NOs: 23-43, for example, having at least about: 90%, 91%, 92%, 93%, 94%, 95%, 96%, 97%, 98%, 99% or 100% sequence identity to any one of SEQ ID NOs: 23-43. In particular embodiments, the enhancer element comprises a nucleotide sequence that is identical to any one of SEQ ID NOs: 23-43.

In some embodiments, the enhancer element is lung cell-specific. In certain embodiments, the enhancer element comprises a nucleotide sequence having at least about 85% sequence identity to any one of SEQ ID NOs: 44-52, for example, having at least about: 90%, 91%, 92%, 93%, 94%, 95%, 96%, 97%, 98%, 99% or 100% sequence identity to any one of SEQ ID NOs: 44-52. In particular embodiments, the enhancer element comprises a nucleotide sequence that is identical to any one of SEQ ID NOs: 44-52.

In some embodiments, the enhancer element is retina cell-specific (e.g., neonatal retina cell-specific). In certain embodiments, the enhancer element comprises a nucleotide sequence having at least about 85% sequence identity to any one of SEQ ID NOs: 53-90, for example, having at least about: 90%, 91%, 92%, 93%, 94%, 95%, 96%, 97%, 98%, 99% or 100% sequence identity to any one of SEQ ID NOs: 53-90. In particular embodiments, the enhancer element comprises a nucleotide sequence that is identical to any one of SEQ ID NOs: 53-90.

In some embodiments, the enhancer element is microglia-specific. In certain embodiments, the enhancer element comprises a nucleotide sequence having at least about 85% sequence identity to any one of SEQ ID NOs: 91-108, for example, having at least about: 90%, 91%, 92%, 93%, 94%, 95%, 96%, 97%, 98%, 99% or 100% sequence identity to any one of SEQ ID NOs: 91-108. In particular embodiments, the enhancer element comprises a nucleotide sequence that is identical to any one of SEQ ID NOs: 91-108.

In some embodiments, the enhancer element is astrocyte-specific. In certain embodiments, the enhancer element comprises a nucleotide sequence having at least about 85% sequence identity to any one of SEQ ID NOs: 109-113, for example, having at least about: 90%, 91%, 92%, 93%, 94%, 95%, 96%, 97%, 98%, 99% or 100% sequence identity to any one of SEQ ID NOs: 109-113. In particular embodiments, the enhancer element comprises a nucleotide sequence that is identical to any one of SEQ ID NOs: 109-113.

In some embodiments, the enhancer element is nucleus accumbens-specific. In certain embodiments, the enhancer element comprises a nucleotide sequence having at least about 85% sequence identity to any one of SEQ ID NOs: 114-126, for example, having at least about: 90%, 91%, 92%, 93%, 94%, 95%, 96%, 97%, 98%, 99% or 100% sequence identity to any one of SEQ ID NOs: 114-126. In particular embodiments, the enhancer element comprises a nucleotide sequence that is identical to any one of SEQ ID NOs: 114-126.

In some embodiments, the enhancer element is cerebral cortex-specific. In certain embodiments, the enhancer element comprises a nucleotide sequence having at least about 85% sequence identity to any one of SEQ ID NOs: 127-160, for example, having at least about: 90%, 91%, 92%, 93%, 94%, 95%, 96%, 97%, 98%, 99% or 100% sequence identity to any one of SEQ ID NOs: 127-160. In particular embodiments, the enhancer element comprises a nucleotide sequence that is identical to any one of SEQ ID NOs: 127-160.

In some embodiments, the enhancer element is glutamatergic neuron-specific. In certain embodiments, the enhancer element comprises a nucleotide sequence having at least about 85% sequence identity to SEQ ID NO: 161 or SEQ ID NO:162, for example, having at least about: 90%, 91%, 92%, 93%, 94%, 95%, 96%, 97%, 98%, 99% or 100% sequence identity to SEQ ID NO: 161 or SEQ ID NO:162. In particular embodiments, the enhancer element comprises a nucleotide sequence that is identical to SEQ ID NO: 161 or SEQ ID NO: 162.

In some embodiments, the enhancer element is GABAergic neuron-specific. In certain embodiments, the enhancer element comprises a nucleotide sequence having at least about 85% sequence identity to SEQ ID NO:163 or SEQ ID NO: 164, for example, having at least about: 90%, 91%, 92%, 93%, 94%, 95%, 96%, 97%, 98%, 99% or 100% sequence identity to SEQ ID NO:163 or SEQ ID NO:164. In particular embodiments, the enhancer element comprises a nucleotide sequence that is identical to SEQ ID NO: 163 or SEQ ID NO: 164.

In some embodiments, the enhancer element is dopaminergic neuron-specific. In certain embodiments, the enhancer element comprises a nucleotide sequence having at least about 85% sequence identity to any one of SEQ ID NOs: 165-169, for example, having at least about: 90%, 91%, 92%, 93%, 94%, 95%, 96%, 97%, 98%, 99% or 100% sequence identity to any one of SEQ ID NOs: 165-169. In particular embodiments, the enhancer element comprises a nucleotide sequence that is identical to any one of SEQ ID NOs: 165-169.

In some embodiments, the enhancer element is cholinergic neuron-specific. In certain embodiments, the enhancer element comprises a nucleotide sequence having at least about 85% sequence identity to SEQ ID NO:170, for example, having at least about: 90%, 91%, 92%, 93%, 94%, 95%, 96%, 97%, 98%, 99% or 100% sequence identity to SEQ ID NO: 170. In particular embodiments, the enhancer element comprises a nucleotide sequence that is identical to SEQ ID NO:170.

In some embodiments, the enhancer element is motor neuron-specific. In certain embodiments, the enhancer element comprises a nucleotide sequence having at least about 85% sequence identity to SEQ ID NO: 171 or SEQ ID NO: 172, for example, having at least about: 90%, 91%, 92%, 93%, 94%, 95%, 96%, 97%, 98%, 99% or 100% sequence identity to SEQ ID NO:171 or SEQ ID NO: 172. In particular embodiments, the enhancer element comprises a nucleotide sequence that is identical to SEQ ID NO: 171 or SEQ ID NO:172.

In some embodiments, the enhancer element is neuron-specific. In certain embodiments, the enhancer element comprises a nucleotide sequence having at least about 85% sequence identity to any one of SEQ ID NOs: 173-176 or SEQ ID NO: 350, for example, having at least about: 90%, 91%, 92%, 93%, 94%, 95%, 96%, 97%, 98%, 99% or 100% sequence identity to any one of SEQ ID NOs: 173-176 or SEQ ID NO: 350. In particular embodiments, the enhancer element comprises a nucleotide sequence that is identical to any one of SEQ ID NOs: 173-176 or SEQ ID NO: 350.

### Transcriptional Control Element

As used herein, “transcriptional control element” refers to a DNA segment that, under cellular conditions (e.g., in a host cell), possesses a transcription-controlling activity (e.g., an activity associated with regulation of transcription initiation), for example, with respect to expression of a target gene. In some embodiments, the transcriptional control element comprises a promoter sequence having at least 80% sequence identity to SEQ ID NO: 1, SEQ ID NO:2, SEQ ID NO:3 or SEQ ID NO:4 operably-linked to a cell- or tissue-specific enhancer element.

In some embodiments, the transcriptional control element has a length of about 700 nucleotides or less, for example, about: 675, 650, 625, 600, 575, 550, 525, 500, 475, 450, 425, 400, 375, 350, 325, 300, 275, 250, 225, 200 nucleotides or less. In some embodiments, the transcriptional control element has a length of about 200 nucleotides to about 700 nucleotides, for example, about: 200-675, 225-675, 225-650, 250-650, 250-625, 275-625, 275-600, 300-600, 300-575, 325-575, 325-550, 350-550, 350-525, 375-525, 375-500, 400-500, 400-475, 425-475 or 425-450 nucleotides.

In some embodiments, the transcriptional control element comprises a nucleotide sequence having at least about 70% sequence identity to any one of SEQ ID NOs: 177-349, for example, having at least about: 75%, 80%, 85%, 90%, 95%, 98%, 99% or 100% sequence identify to any one of SEQ ID NOs: 177-349. In certain embodiments, the transcriptional control element comprises a nucleotide sequence that is identical to any one of SEQ ID NOs: 177-349.

In some embodiments, the transcriptional control element comprises a nucleotide sequence having at least about 70% sequence identity to any one of SEQ ID NOs: 177-194, SEQ ID NOs: 195-215, SEQ ID NOs: 216-224, SEQ ID NOs: 225-262, SEQ ID NOs: 263-280, SEQ ID NOs: 281-285, SEQ ID NOs: 286-298, SEQ ID NOs: 299-332, SEQ ID NO:333 or SEQ ID NO:334, SEQ ID NO:335 or SEQ ID NO:336, SEQ ID NOs: 337-341, SEQ ID NO: 342, SEQ ID NO:343 or SEQ ID NO:344, or SEQ ID NOs: 345-349, for example, having at least about: 75%, 80%, 85%, 90%, 95%, 98%, 99% or 100% sequence identify to any one of SEQ ID NOs: 177-194, SEQ ID NOs: 195-215, SEQ ID NOs: 216-224, SEQ ID NOs: 225-262, SEQ ID NOs: 263-280, SEQ ID NOs: 281-285, SEQ ID NOs: 286-298, SEQ ID NOs: 299-332, SEQ ID NO:333 or SEQ ID NO:334, SEQ ID NO:335 or SEQ ID NO:336, SEQ ID NOs: 337-341, SEQ ID NO:342, SEQ ID NO:343 or SEQ ID NO:344, or SEQ ID NOs: 345-349.

In certain embodiments, the transcriptional control element comprises a nucleotide sequence that is identical to any one of SEQ ID NOs: 177-194, SEQ ID NOs: 195-215, SEQ ID NOs: 216-224, SEQ ID NOs: 225-262, SEQ ID NOs: 263-280, SEQ ID NOs: 281-285, SEQ ID NOs: 286-298, SEQ ID NOs: 299-332, SEQ ID NO:333 or SEQ ID NO:334, SEQ ID NO: 335 or SEQ ID NO:336, SEQ ID NOs: 337-341, SEQ ID NO:342, SEQ ID NO:343 or SEQ ID NO:344, or SEQ ID NOs: 345-349.

In some embodiments, the transcriptional control element comprises a nucleotide sequence having at least about 70% sequence identity to any one of SEQ ID NOs: 177-194, for example, having at least about: 75%, 80%, 85%, 90%, 95%, 98%, 99% or 100% sequence identify to any one of SEQ ID NOs: 177-194. In certain embodiments, the transcriptional control element comprises a nucleotide sequence that is identical to any one of SEQ ID NOs: 177-194.

In some embodiments, the transcriptional control element comprises a nucleotide sequence having at least about 70% sequence identity to any one of SEQ ID NOs: 195-215, for example, having at least about: 75%, 80%, 85%, 90%, 95%, 98%, 99% or 100% sequence identify to any one of SEQ ID NOs: 195-215. In certain embodiments, the transcriptional control element comprises a nucleotide sequence that is identical to any one of SEQ ID NOs: 195-215.

In some embodiments, the transcriptional control element comprises a nucleotide sequence having at least about 70% sequence identity to any one of SEQ ID NOs: 216-224, for example, having at least about: 75%, 80%, 85%, 90%, 95%, 98%, 99% or 100% sequence identify to any one of SEQ ID NOs: 216-224. In certain embodiments, the transcriptional control element comprises a nucleotide sequence that is identical to any one of SEQ ID NOs: 216-224.

In some embodiments, the transcriptional control element comprises a nucleotide sequence having at least about 70% sequence identity to any one of SEQ ID NOs: 225-262, for example, having at least about: 75%, 80%, 85%, 90%, 95%, 98%, 99% or 100% sequence identify to any one of SEQ ID NOs: 225-262. In certain embodiments, the transcriptional control element comprises a nucleotide sequence that is identical to any one of SEQ ID NOs: 225-262.

In some embodiments, the transcriptional control element comprises a nucleotide sequence having at least about 70% sequence identity to any one of SEQ ID NOs: 263-280, for example, having at least about: 75%, 80%, 85%, 90%, 95%, 98%, 99% or 100% sequence identify to any one of SEQ ID NOs: 263-280. In certain embodiments, the transcriptional control element comprises a nucleotide sequence that is identical to any one of SEQ ID NOs: 263-280.

In some embodiments, the transcriptional control element comprises a nucleotide sequence having at least about 70% sequence identity to any one of SEQ ID NOs: 281-285, for example, having at least about: 75%, 80%, 85%, 90%, 95%, 98%, 99% or 100% sequence identify to any one of SEQ ID NOs: 281-285. In certain embodiments, the transcriptional control element comprises a nucleotide sequence that is identical to any one of SEQ ID NOs: 281-285.

In some embodiments, the transcriptional control element comprises a nucleotide sequence having at least about 70% sequence identity to any one of SEQ ID NOs: 286-298, for example, having at least about: 75%, 80%, 85%, 90%, 95%, 98%, 99% or 100% sequence identify to any one of SEQ ID NOs: 286-298. In certain embodiments, the transcriptional control element comprises a nucleotide sequence that is identical to any one of SEQ ID NOs: 286-298.

In some embodiments, the transcriptional control element comprises a nucleotide sequence having at least about 70% sequence identity to any one of SEQ ID NOs: 299-332, for example, having at least about: 75%, 80%, 85%, 90%, 95%, 98%, 99% or 100% sequence identify to any one of SEQ ID NOs: 299-332. In certain embodiments, the transcriptional control element comprises a nucleotide sequence that is identical to any one of SEQ ID NOs: 299-332.

In some embodiments, the transcriptional control element comprises a nucleotide sequence having at least about 70% sequence identity to SEQ ID NO:333 or SEQ ID NO: 334, for example, having at least about: 75%, 80%, 85%, 90%, 95%, 98%, 99% or 100% sequence identify to SEQ ID NO:333 or SEQ ID NO:334. In certain embodiments, the transcriptional control element comprises a nucleotide sequence that is identical to SEQ ID NO: 333 or SEQ ID NO:334.

In some embodiments, the transcriptional control element comprises a nucleotide sequence having at least about 70% sequence identity to SEQ ID NO:335 or SEQ ID NO: 336, for example, having at least about: 75%, 80%, 85%, 90%, 95%, 98%, 99% or 100% sequence identify to SEQ ID NO:335 or SEQ ID NO:336. In certain embodiments, the transcriptional control element comprises a nucleotide sequence that is identical to SEQ ID NO: 335 or SEQ ID NO:336.

In some embodiments, the transcriptional control element comprises a nucleotide sequence having at least about 70% sequence identity to any one of SEQ ID NOs: 337-341, for example, having at least about: 75%, 80%, 85%, 90%, 95%, 98%, 99% or 100% sequence identify to any one of SEQ ID NOs: 337-341. In certain embodiments, the transcriptional control element comprises a nucleotide sequence that is identical to any one of SEQ ID NOs: 337-341.

In some embodiments, the transcriptional control element comprises a nucleotide sequence having at least about 70% sequence identity to SEQ ID NO:342, for example, having at least about: 75%, 80%, 85%, 90%, 95%, 98%, 99% or 100% sequence identify to SEQ ID NO:342. In certain embodiments, the transcriptional control element comprises a nucleotide sequence that is identical to SEQ ID NO:342.

In some embodiments, the transcriptional control element comprises a nucleotide sequence having at least about 70% sequence identity to SEQ ID NO:343 or SEQ ID NO: 344, for example, having at least about: 75%, 80%, 85%, 90%, 95%, 98%, 99% or 100% sequence identify to SEQ ID NO:343 or SEQ ID NO:344. In certain embodiments, the transcriptional control element comprises a nucleotide sequence that is identical to SEQ ID NO: 343 or SEQ ID NO:344.

In some embodiments, the transcriptional control element comprises a nucleotide sequence having at least about 70% sequence identity to any one of SEQ ID NOs: 345-349, for example, having at least about: 75%, 80%, 85%, 90%, 95%, 98%, 99% or 100% sequence identify to any one of SEQ ID NOs: 345-349. In certain embodiments, the transcriptional control element comprises a nucleotide sequence that is identical to any one of SEQ ID NOs: 345-349.

In some embodiments, the transcriptional control element further comprises one or more inducer elements, silencer elements, 5′ untranslated regions (UTRs), 3′UTRs, terminator elements, CAAT boxes, CCAAT boxes, Pribnow boxes, SECIS elements, polyadenylation signals, A-boxes, Z-boxes, C-boxes, E-boxes, G-boxes, Cis-regulatory elements (CREs), or a combination thereof.

### Target Genes

In some embodiments, a polynucleotide of the disclosure comprises a coding sequence for a target gene operably linked to the transcriptional control element.

As used herein, the phrase “operably linked” means that the nucleic acid is positioned in the polynucleotide, e.g., vector, in such a way that enables expression of the nucleic acid under control of the element (e.g., promoter) to which it is linked.

In certain embodiments, the coding sequence for the target gene comprises at least about 3 kilobases, for example, at least about: 3.1, 3.2, 3.3, 3.4, 3.5, 3.6, 3.7, 3.8, 3.9, 4.0, 4.1, 4.2, 4.3 or 4.4 kilobases. In certain embodiments, the coding sequence for the target gene comprises about 3.0-4.5 kilobases, for example, at least about: 3.2-4.5, 3.2-4.4, 3.4-4.4, 3.4-4.3, 3.5-4.3, 3.5-4.2, 3.6-4.2, 3.6-4.1, 3.7-4.1, 3.7-4.0, 3.8-4.0 or 3.8-3.9 kilobases. In particular embodiments, the coding sequence for the target gene comprises at least about 4 kilobases.

In some embodiments, the polynucleotide comprises two or more coding sequences for two or more target genes operably linked to the transcriptional control element. In certain embodiments, the sum of the two or more coding sequences comprises at least about 3 kilobases, for example, at least about: 3.1, 3.2, 3.3, 3.4, 3.5, 3.6, 3.7, 3.8, 3.9, 4.0, 4.1, 4.2, 4.3 or 4.4 kilobases. In certain embodiments, the two or more coding sequences comprise about 3.0-4.5 kilobases, for example, at least about: 3.2-4.5, 3.2-4.4, 3.4-4.4, 3.4-4.3, 3.5-4.3, 3.5-4.2, 3.6-4.2, 3.6-4.1, 3.7-4.1, 3.7-4.0, 3.8-4.0 or 3.8-3.9 kilobases. In particular embodiments, the two or more coding sequences comprise at least about 4 kilobases.

In some embodiments, the polynucleotide comprises one or more cloning sites for insertion of a coding sequence for a target gene. In some embodiments, the polynucleotide comprises one or more cloning sites in place of the target gene. As used herein, a “cloning site” refers to a short segment of nucleotides in the vector that contain one or more unique restriction sites that allow for insertion of a nucleotide “target gene” or “gene of interest” into the vector.

### Additional Elements

In some embodiments, the polynucleotide further comprises an inducer element. Non-limiting examples of inducer elements include: Cre-Lox, Flp-Frt, Cre-ER, Flp-ER, DIO (double-floxed inverse ORF), Tet-On/Off (tetracycline-inducible), tTA (tetracycline transactivator), rtTA (reverse tetracycline transactivator), optogenetic elements, chemogenetic elements, a riboswitch, small molecule-binding elements, and nanobody-binding elements, among others.

In some embodiments, the polynucleotide further comprises a selectable marker element.

As used herein, a “selectable marker element” is an element that confers a trait suitable for artificial selection. Examples of selectable marker elements useful in the present disclosure include, but are not limited to, beta-lactamase, neomycin resistance genes, mutant Fabl genes conferring triclosan resistance, URA3 elements, fluorescent gene products, affinity tags such as GST, His, CBP, MBP, and epitope tags such as Myc HA, FLAG. Selectable marker elements can be negative or positive selection markers.

### Vectors

In some embodiments, the polynucleotide is a vector.

A “vector” is a nucleic acid molecule which may be employed to introduce a nucleic acid sequence or gene into a cell, either in vitro, ex vivo, or in vivo.

In some embodiments, the vector is a non-viral vector. Non-limiting examples of non-viral vectors include: linear DNA, circular DNA, plasmid DNA, linear RNA, circRNA, mRNA, siRNA, shRNA, miRNA, among others. Such non-viral vectors can be delivered to a cell by physical (e.g., electroporation, sonoporation, ultrasound, photoporation, magnetofection, hydroporation, gene gun, microinjection) or chemical (e.g., lipofection, lipoplexes, dendrimers, polymers, polyplexes, solid lipid nanoparticles, liposomes, exosomes, vesicles, synthetic nanoparticles, lipid nanoparticles, cell-penetrating peptides) methods known in the art.

In other embodiments, the vector is a viral vector. Non-limiting examples of viral vectors include adeno-associated virus (AAV) vectors, adenovirus vectors, anellovirus vectors, coronavirus vectors, herpes virus vectors, lentivirus vectors, polyomavirus vectors, rabies virus vectors, recombinant simian virus 40 vectors, reovirus vectors, retrovirus vectors, rhinovirus vectors, sindbis virus vectors, vaccinia virus vectors, vesicular stomatitis virus vectors, semliki forest virus vectors and yellow fever virus vectors.

In some embodiments, the viral vector (e.g., AAV vector) is a gene therapy vector.

In certain embodiments, the viral vector is a recombinant AAV vector. A “recombinant AAV vector” refers to a polynucleotide vector comprising one or more heterologous sequences flanked by at least one AAV inverted terminal repeat sequence (ITR). Recombinant AAV vectors can be replicated and packaged into infectious viral particles when present in a host cell that is expressing suitable helper functions (e.g., has been infected with a suitable helper virus or transfected with helper genes), AAV rep and cap gene products. Recombinant AAV vectors include plasmids and plasmid fragments encapsulated in a viral particle, e.g., an AAV particle. A rAAV vector can be packaged into an AAV virus capsid to generate a recombinant adeno-associated viral particle (rAAV particle).

In some embodiments, the recombinant AAV vector is a plasmid. In certain embodiments, the recombinant AAV vector is a linear artificial chromosome.

### Viruses

In another aspect, the present disclosure provides a virus comprising one or more of the polynucleotides or vectors described herein. In some embodiments, the virus is AAV, adenovirus, anellovirus, coronavirus, herpes virus, lentivirus, polyomavirus, rabies virus, recombinant simian virus 40, reovirus, retrovirus, rhinovirus, sindbis virus, vaccinia virus, vesicular stomatitis virus, semliki forest virus, yellow fever virus or a combination thereof. In some embodiments, the virus is an AAV.

### Host Cells

In another aspect, the present disclosure provides a host cell comprising one or more of the polynucleotides (e.g., vectors) or viruses described herein.

In some embodiments, the host cell is a mammalian cell.

In certain embodiments, the host cell is a myoblast, a pancreatic cell, a lung cell, a neonatal retina cell, a microglia cell, an astrocyte or a neuron.

In some embodiments, the host cell is an in vitro cell.

In certain embodiments, the host cell is an ex vivo cell.

In some embodiments, the host cell is a cell of a subject. The terms “subject” and “patient” can be used interchangeably herein. “Patient in need thereof” or “subject in need thereof” refers to a mammalian subject, preferably human, who has been diagnosed with or is suspected of having a disease. “Patient in need thereof” or “subject in need thereof” includes those subjects already with the undesired physiological change or disease well as those subjects prone to have the physiological change or disease.

In certain embodiments, the host cell is a myoblast, a pancreatic cell, a lung cell, a neonatal retina cell, a microglia cell, an astrocyte or a neuron.

In particular embodiments, the neuron is a neuron of the nucleus accumbens, a neuron of the cerebral cortex, a glutamatergic neuron, a GABAergic neuron, a dopaminergic neuron, a cholinergic neuron, or a motor neuron.

In some embodiments, the host cell is a cell of a subject and the enhancer element comprises a nucleotide sequence having at least about 85% sequence identity to any one of SEQ ID NOs: 5-22, SEQ ID NOs: 23-43, SEQ ID NOs: 44-52, SEQ ID NOs: 53-90, SEQ ID NOs: 91-108, SEQ ID NOs: 109-113, SEQ ID NOs: 114-126, SEQ ID NOs: 127-160, SEQ ID NO: 161 or SEQ ID NO:162, SEQ ID NO: 163 or SEQ ID NO: 164, SEQ ID NOs: 165-169, SEQ ID NO: 170, SEQ ID NO:171 or SEQ ID NO: 172, or SEQ ID NOs: 173-176, or SEQ ID NO: 350, for example, having at least about: 90%, 91%, 92%, 93%, 94%, 95%, 96%, 97%, 98%, 99% or 100% sequence identity to any one of SEQ ID NOs: 5-22, SEQ ID NOs: 23-43, SEQ ID NOs: 44-52, SEQ ID NOs: 53-90, SEQ ID NOs: 91-108, SEQ ID NOs: 109-113, SEQ ID NOs: 114-126, SEQ ID NOs: 127-160, SEQ ID NO:161 or SEQ ID NO:162, SEQ ID NO: 163 or SEQ ID NO: 164, SEQ ID NOs: 165-169, SEQ ID NO: 170, SEQ ID NO: 171 or SEQ ID NO: 172, or SEQ ID NOs: 173-176, or SEQ ID NO: 350.

In some embodiments, the host cell is a myoblast of gastrocnemius muscle, and the enhancer sequence comprises a nucleotide sequence having at least 85% sequence identity to any one of SEQ ID NOs: 5-22, for example, having at least about: 90%, 91%, 92%, 93%, 94%, 95%, 96%, 97%, 98%, 99% or 100% sequence identity to any one of SEQ ID NOs: 5-22.

In some embodiments, the host cell is a pancreatic cell, and the enhancer sequence comprises a nucleotide sequence having at least 85% sequence identity to any one of SEQ ID NOs: 23-43, for example, having at least about: 90%, 91%, 92%, 93%, 94%, 95%, 96%, 97%, 98%, 99% or 100% sequence identity to any one of SEQ ID NOs: 23-43.

In some embodiments, the host cell is a lung cell, and the enhancer sequence comprises a nucleotide sequence having at least 85% sequence identity to any one of SEQ ID NOs: 44-52, for example, having at least about: 90%, 91%, 92%, 93%, 94%, 95%, 96%, 97%, 98%, 99% or 100% sequence identity to any one of SEQ ID NOs: 44-52.

In some embodiments, the host cell is a neonatal retina cell, and the enhancer sequence comprises a nucleotide sequence having at least 85% sequence identity to any one of SEQ ID NOs: 53-90, for example, having at least about: 90%, 91%, 92%, 93%, 94%, 95%, 96%, 97%, 98%, 99% or 100% sequence identity to any one of SEQ ID NOs: 53-90.

In some embodiments, the host cell is a microglia cell, and the enhancer sequence comprises a nucleotide sequence having at least 85% sequence identity to any one of SEQ ID NOs: 91-108, for example, having at least about: 90%, 91%, 92%, 93%, 94%, 95%, 96%, 97%, 98%, 99% or 100% sequence identity to any one of SEQ ID NOs: 91-108.

In some embodiments, the host cell is an astrocyte, and the enhancer sequence comprises a nucleotide sequence having at least 85% sequence identity to any one of SEQ ID NOs: 109-113, for example, having at least about: 90%, 91%, 92%, 93%, 94%, 95%, 96%, 97%, 98%, 99% or 100% sequence identity to any one of SEQ ID NOs: 109-113.

In some embodiments, the host cell is a neuron of the nucleus accumbens, and the enhancer sequence comprises a nucleotide sequence having at least 85% sequence identity to any one of SEQ ID NOs: 114-126, for example, having at least about: 90%, 91%, 92%, 93%, 94%, 95%, 96%, 97%, 98%, 99% or 100% sequence identity to any one of SEQ ID NOs: 114-126.

In some embodiments, the host cell is a neuron of the cerebral cortex, and the enhancer sequence comprises a nucleotide sequence having at least 85% sequence identity to any one of SEQ ID NOs: 127-160, for example, having at least about: 90%, 91%, 92%, 93%, 94%, 95%, 96%, 97%, 98%, 99% or 100% sequence identity to any one of SEQ ID NOs: 127-160.

In some embodiments, the host cell is a glutamatergic neuron, and the enhancer sequence comprises a nucleotide sequence having at least 85% sequence identity to SEQ ID NO: 161 or SEQ ID NO: 162, for example, having at least about: 90%, 91%, 92%, 93%, 94%, 95%, 96%, 97%, 98%, 99% or 100% sequence identity to SEQ ID NO:161 or SEQ ID NO: 162.

In some embodiments, the host cell is a GABAergic neuron, and the enhancer sequence comprises a nucleotide sequence having at least 85% sequence identity to SEQ ID NO: 163 or SEQ ID NO:164, for example, having at least about: 90%, 91%, 92%, 93%, 94%, 95%, 96%, 97%, 98%, 99% or 100% sequence identity to SEQ ID NO:163 or SEQ ID NO: 164.

In some embodiments, the host cell is a dopaminergic neuron, and the enhancer sequence comprises a nucleotide sequence having at least 85% sequence identity to any one of SEQ ID NOs: 165-169, for example, having at least about: 90%, 91%, 92%, 93%, 94%, 95%, 96%, 97%, 98%, 99% or 100% sequence identity to any one of SEQ ID NOs: 165-169.

In some embodiments, the host cell is a cholinergic neuron, and the enhancer sequence comprises a nucleotide sequence having at least 85% sequence identity to SEQ ID NO: 170, for example, having at least about: 90%, 91%, 92%, 93%, 94%, 95%, 96%, 97%, 98%, 99% or 100% sequence identity to SEQ ID NO:170.

In some embodiments, the host cell is a motor neuron, and the enhancer sequence comprises a nucleotide sequence having at least 85% sequence identity to SEQ ID NO:171 or SEQ ID NO: 172, for example, having at least about: 90%, 91%, 92%, 93%, 94%, 95%, 96%, 97%, 98%, 99% or 100% sequence identity to SEQ ID NO:171 or SEQ ID NO:172.

In some embodiments, the host cell is a neuron, and the enhancer sequence comprises a nucleotide sequence having at least 85% sequence identity to any one of SEQ ID NOs: 173-176 or SEQ ID NO: 350, for example, having at least about: 90%, 91%, 92%, 93%, 94%, 95%, 96%, 97%, 98%, 99% or 100% sequence identity to any one of SEQ ID NOs: 173-176 or SEQ ID NO: 350.

In some embodiments, the transcriptional control element comprises a nucleotide sequence having at least about 70% sequence identity to any one of SEQ ID NOs: 177-349, for example, having at least about: 75%, 80%, 85%, 90%, 95%, 98%, 99% or 100% sequence identity to any one of SEQ ID NOs: 177-349.

In some embodiments, the transcriptional control element comprises a nucleotide sequence identical to any one of SEQ ID NOs: 177-349.

In some embodiments, the host cell is a myoblast of gastrocnemius muscle, and the transcriptional control element comprises a nucleotide sequence having at least 70% sequence identity to any one of SEQ ID NOs: 177-194, for example, having at least about: 75%, 80%, 85%, 90%, 95%, 98%, 99% or 100% sequence identity to any one of SEQ ID NOs: 177-194.

In some embodiments, the host cell is a pancreatic cell, and the transcriptional control element comprises a nucleotide sequence having at least 70% sequence identity to any one of SEQ ID NOs: 195-215, for example, having at least about: 75%, 80%, 85%, 90%, 95%, 98%, 99% or 100% sequence identity to any one of SEQ ID NOs: 195-215.

In some embodiments, the host cell is a lung cell, and the transcriptional control element comprises a nucleotide sequence having at least 70% sequence identity to any one of SEQ ID NOs: 216-224, for example, having at least about: 75%, 80%, 85%, 90%, 95%, 98%, 99% or 100% sequence identity to any one of SEQ ID NOs: 216-224.

In some embodiments, the host cell is a neonatal retina cell, and the transcriptional control element comprises a nucleotide sequence having at least 70% sequence identity to any one of SEQ ID NOs: 225-262, for example, having at least about: 75%, 80%, 85%, 90%, 95%, 98%, 99% or 100% sequence identity to any one of SEQ ID NOs: 225-262.

In some embodiments, the host cell is a microglia cell, and the transcriptional control element comprises a nucleotide sequence having at least 70% sequence identity to any one of SEQ ID NOs: 263-280, for example, having at least about: 75%, 80%, 85%, 90%, 95%, 98%, 99% or 100% sequence identity to any one of SEQ ID NOs: 263-280.

In some embodiments, the host cell is an astrocyte, and the transcriptional control element comprises a nucleotide sequence having at least 70% sequence identity to any one of SEQ ID NOs: 281-285, for example, having at least about: 75%, 80%, 85%, 90%, 95%, 98%, 99% or 100% sequence identity to any one of SEQ ID NOs: 281-285.

In some embodiments, the host cell is a neuron of the nucleus accumbens, and the transcriptional control element comprises a nucleotide sequence having at least 70% sequence identity to any one of SEQ ID NOs: 286-298, for example, having at least about: 75%, 80%, 85%, 90%, 95%, 98%, 99% or 100% sequence identity to any one of SEQ ID NOs: 286-298.

In some embodiments, the host cell is a neuron of the cerebral cortex, and the transcriptional control element comprises a nucleotide sequence having at least 70% sequence identity to any one of SEQ ID NOs: 299-332, for example, having at least about: 75%, 80%, 85%, 90%, 95%, 98%, 99% or 100% sequence identity to any one of SEQ ID NOs: 299-332.

In some embodiments, the host cell is a glutamatergic neuron, and the transcriptional control element comprises a nucleotide sequence having at least 70% sequence identity to SEQ ID NO:333 or SEQ ID NO:334, for example, having at least about: 75%, 80%, 85%, 90%, 95%, 98%, 99% or 100% sequence identity to SEQ ID NO:333 or SEQ ID NO:334.

In some embodiments, the host cell is a GABAergic neuron, and the transcriptional control element comprises a nucleotide sequence having at least 70% sequence identity to SEQ ID NO:335 or SEQ ID NO:336, for example, having at least about: 75%, 80%, 85%, 90%, 95%, 98%, 99% or 100% sequence identity to SEQ ID NO:335 or SEQ ID NO:336.

In some embodiments, the host cell is a dopaminergic neuron, and the transcriptional control element comprises a nucleotide sequence having at least 70% sequence identity to any one of SEQ ID NOs: 337-341, for example, having at least about: 75%, 80%, 85%, 90%, 95%, 98%, 99% or 100% sequence identity to any one of SEQ ID NOs: 337-341.

In some embodiments, the host cell is a cholinergic neuron, and the transcriptional control element comprises a nucleotide sequence having at least 70% sequence identity to SEQ ID NO:342, for example, having at least about: 75%, 80%, 85%, 90%, 95%, 98%, 99% or 100% sequence identity to SEQ ID NO:342.

In some embodiments, the host cell is a motor neuron, and the transcriptional control element comprises a nucleotide sequence having at least 70% sequence identity to SEQ ID NO:343 or SEQ ID NO:344, for example, having at least about: 75%, 80%, 85%, 90%, 95%, 98%, 99% or 100% sequence identity to SEQ ID NO:343 or SEQ ID NO:344.

In some embodiments, the host cell is a neuron, and the transcriptional control element comprises a nucleotide sequence having at least 70% sequence identity to any one of SEQ ID NOs: 345-349, for example, having at least about: 75%, 80%, 85%, 90%, 95%, 98%, 99% or 100% sequence identity to any one of SEQ ID NOs: 345-349.

In some embodiments, a polynucleotide of the disclosure is introduced into the host cell by a physical method, for example, by electroporation, gene gun, hydroporation, magnetofection, microinjection, photoporation, sonoporation or ultrasound. In certain embodiments, a polynucleotide of the disclosure is introduced into the host cell by a chemical method, for example, via dendrimers, exosomes, lipid nanoparticles lipofection, lipoplexes, liposomes, polymers, polyplexes, solid lipid nanoparticles, synthetic nanoparticles or vesicles.

In some embodiments, a virus of the disclosure is introduced into a host cell by infection.

### Pharmaceutical Compositions

In another aspect, the present disclosure provides a pharmaceutical composition comprising one or more of the polynucleotides, viruses or host cells described herein, and one or more pharmaceutically acceptable carriers, excipients, stabilizers, diluents or tonifiers (Remington's Pharmaceutical Sciences 16th edition, Osol, A. Ed. (1980)). Suitable pharmaceutically acceptable carriers, excipients, or stabilizers are nontoxic to recipients at the dosages and concentrations employed. Non-limiting examples of pharmaceutically acceptable carriers, excipients, stabilizers, diluents or tonifiers include buffers (e.g., phosphate, citrate, histidine), antioxidants (e.g., ascorbic acid or methionine), preservatives, proteins (e.g., serum albumin, gelatin or immunoglobulins); hydrophilic polymers, amino acids, carbohydrates (e.g., monosaccharides, disaccharides, glucose, mannose or dextrins); chelating agents (e.g., EDTA), sugars (e.g., sucrose, mannitol, trehalose or sorbitol), salt-forming counter-ions (e.g., sodium), metal complexes (e.g., Zn-protein complexes); non-ionic surfactants (e.g., Tween), PLURONICS™ and polyethylene glycol (PEG).

In some embodiments, the pharmaceutical composition is formulated for a suitable administration schedule and route. Non-limiting examples of administration routes include intra-arterial, intracameral, intracisternal, intradermal, intradiaphragmatic, intramuscular, intranasal, intraperitoneal, intrapleural, intraportal, intrathecal, intravenous, intravitreal, pancreatic intraductal, retro-orbital, subconjunctival, subcutaneous, subretinal, and sub-Tenon. In certain embodiments, the pharmaceutical composition is formulated for infusion (e.g., intravenous infusion). In some embodiments, the pharmaceutical composition is formulated for intrathecal injection. In particular embodiments, the intrathecal injection is an intrathecal lumbar injection.

In some embodiments, the pharmaceutical composition is formulated to be administered with a second therapeutic agent as a combination therapy.

In some embodiments, the pharmaceutical composition is stored in the form of an aqueous solution or a dried formulation (e.g., lyophilized).

### Methods of Target Gene Expression

In another aspect, the present disclosure provides a method of expressing a target gene in any one of the host cells described herein, wherein the method comprises contacting the host cell with any one of the polynucleotides or viruses described herein under conditions whereby the polynucleotide or the virus is introduced into the host cell, and expression of the target gene occurs in the host cell.

In some embodiments, the host cell is a mammalian cell, for example a human cell.

In some embodiments, the host cell is an in vitro cell. In other embodiments, the host cell is an ex vivo cell.

In particular embodiments, the host cell is a cell of a subject.

In some embodiments, the cell of the subject is allogeneic. In other embodiments, the cell of the subject is autologous or syngeneic.

In some embodiments, the polynucleotide is a non-viral vector. In certain embodiments, the non-viral vector is introduced into the host cell by a physical method, for example, by electroporation, gene gun, hydroporation, magnetofection, microinjection, photoporation, sonoporation or ultrasound. In certain embodiments, the non-viral vector is introduced into the host cell by a chemical method, for example, via dendrimers, exosomes, lipid nanoparticles lipofection, lipoplexes, liposomes, polymers, polyplexes, solid lipid nanoparticles, synthetic nanoparticles or vesicles.

In another aspect, the present disclosure provides a method of expressing a target gene in a subject in need thereof, wherein the method comprises administering to the subject an effective amount of any one of the polynucleotides, viruses or pharmaceutical compositions described herein, whereby expression of the target gene occurs in a host cell of the subject.

In some embodiments, the host cell is a myoblast, a pancreatic cell, a lung cell, a neonatal retina cell, a microglia cell, an astrocyte or a neuron.

In certain embodiments, the target gene is expressed in a neuron of the subject. In particular embodiments, the neuron is a neuron of the nucleus accumbens, a neuron of the cerebral cortex, a glutamatergic neuron, a GABAergic neuron, a dopaminergic neuron, a cholinergic neuron, or a motor neuron.

In some embodiments, the subject is a human.

In some embodiments, the subject is 18 years of age or older, e.g., 18 to less than 40 years of age, 18 to less than 45 years of age, 18 to less than 50 years of age, 18 to less than 55 years of age, 18 to less than 60 years of age, 18 to less than 65 years of age, 18 to less than 70 years of age, 18 to less than 75 years of age, 40 to less than 75 years of age, 45 to less than 75 years of age, 50 to less than 75 years of age, 55 to less than 75 years of age, 60 to less than 75 years of age, 65 to less than 75 years of age, 60 to less than 75 years of age, 40 years of age or older, 45 years of age or older, 50 years of age or older, 55 years of age or older, 60 years of age or older, 65 years of age or older, 70 years of age or older or 75 years of age or older.

In some embodiments, the subject is a child. In some embodiments, the subject is 18 years of age or younger, e.g., 0-18 years of age, 0-12 years of age, 0-16 years of age, 0-17 years of age, 2-12 years of age, 2-16 years of age, 2-17 years of age, 2-18 years of age, 3-12 years of age, 3-16 years of age, 3-17 years of age, 3-18 years of age, 4-12 years of age, 4-16 years of age, 4-17 years of age, 4-18 years of age, 6-12 years of age, 6-16 years of age, 6-17 years of age, 6-18 years of age, 9-12 years of age, 9-16 years of age, 9-17 years of age, 9-18 years of age, 12-16 years of age, 12-17 years of age or 12-18 years of age.

In some embodiments, the subject has a condition selected from the group consisting of: Alzheimer's disease (AD), amyotrophic lateral sclerosis (ALS), ataxia telangietacsia, cancer, Dopa-responsive dystonia, a fatty liver disease, Fragile X syndrome, Friedreich ataxia, Giant axonal neuropathy, glycogen storage disease type 2 (Pompe), Huntington's disease (HD), Krabbe disease, multiple sclerosis (MS), multiple system atrophy (MSA), neuronal ceroid lipofuscinosis (Batten disease) Niemann-Pick disease A, B and C, pain, Parkinson's disease (PD), Pelizaeus-Merzbacher disease, primary lateral sclerosis, a prion disease, proximal myotonic myopathy, spinal muscular atrophy (SMA), Spinocerebellar ataxia type 1, 2 and 3, Tay-Says disease, and X-linked adrenoleukodystrophy.

In some embodiments, the subject has α-1 Fabry disease, adrenoleukodystrophy, antitrypsin deficiency, Becker muscular dystrophy, β-thalassaemia, Canavan disease, chronic granulomatous disease, Duchenne muscular dystrophy (DMD), familial adenomatous polyposis, familial hypercholesterolaemia, Fanconi anaemia, galactosialidosis, Gaucher's disease, gyrate atrophy, hemophilia A, hemophilia B, Hurler syndrome, Hunter syndrome, Huntington's chorea, junctional epidermolysis bullosa, late infantile neuronal ceroid lipofuscinosis, Leber congenital amaurosis (LCA), leukocyte adherence deficiency, limb-girdle muscular dystrophy (LGMD), lipoprotein lipase deficiency, mucopolysaccharidosis type VII, Ornithine transcarbamylase deficiency, Pompe Disease, Purine nucleoside phosphorylase deficiency, recessive dystrophic epidermolysis bullosa, Sickle cell disease; Tay Sachs disease, Wiskott-Aldrich syndrome, X-linked myotubular myopathy or X-linked severe combined immunodeficiency (SCID-X1), and the method expresses the target gene in a myoblast.

In some embodiments, the subject has diabetes mellitus, pancreatic adenocarcinoma, pancreatic cancer or pancreatitis, and the method expresses the target gene in a pancreas cell.

In some embodiments, the subject has acute lung injury (ALI), acute respiratory distress syndrome (ARDS), alpha-1 antitrypsin deficiency, asthma, chronic obstructive pulmonary disease (COPD), cystic fibrosis, idiopathic pulmonary fibrosis, lung cancer, pneumoconiosis, primary ciliary dyskinesia (PCD) or pulmonary arterial hypertension (PAH), and the method expresses the target gene in a lung cell.

In some embodiments, the subject has Alzheimer's disease, amyotrophic lateral sclerosis (ALS), autoimmune encephalomyelitis, depression, frontotemporal dementia, metachromatic leukodystrophy (MLD), multiple sclerosis, cancer or Parkinson's disease, and the method expresses the target gene in microglia. In certain embodiments, the cancer is glioblastoma.

In some embodiments, the subject has Alexander diseases, Alzheimer's disease, amyotrophic lateral sclerosis (ALS), cancer, Huntington disease (HD), Korsakoff-Wernicke syndrome, a major depressive disorder (MDD), schizophrenia, stroke, temporal lobe epilepsy or toxic encephalopathies, and the method expresses the target gene in an astrocyte. In certain embodiments, the cancer is anaplastic astrocytoma, glioblastoma or gliosarcoma.

In some embodiments, the subject has bipolar disorder, major depressive disorder (MDD) or schizophrenia, and the method expresses the target gene in a nucleus accumbens neuron.

In some embodiments, the subject has Alzheimer's disease (AD), Huntington disease (HD), Parkinson's disease or Pick disease, and the method expresses the target gene in a cerebral cortex neuron.

In some embodiments, the subject has Alzheimer's disease, autism spectrum disorder, amyotrophic lateral sclerosis (ALS), bipolar disorder, epilepsy, Huntington's disease, a major depressive disorder (MDD) or Parkinson's disease or schizophrenia, and the method expresses the target gene in a glutamatergic neuron.

In some embodiments, the subject has Alzheimer's disease, Angelman syndrome, autism spectrum disorder, epilepsy, GABA-transaminase deficiency, major depressive disorder (MDD), Rett syndrome, schizophrenia, succinate semialdehyde dehydrogenase (SSADH) deficiency, Tourette's syndrome or X syndrome (FXS), and the method expresses the target gene in a GABAergic neuron.

In some embodiments, the subject has amyotrophic lateral sclerosis (ALS), bipolar disorder, chronic hyperprolactinemia or Parkinson's disease, and the method expresses the target gene in a dopaminergic neuron.

In some embodiments, the subject has Alzheimer's disease, Huntington's disease (HD), multiple sclerosis or Parkinson's disease, and the method expresses the target gene in a cholinergic neuron.

In some embodiments, the subject has amyotrophic lateral sclerosis (ALS), Friedreich ataxia, giant axonal neuropathy, Kennedy's disease, post-herpetic neuralgia, primary lateral sclerosis (PLS), progressive bulbar palsy (PBP), spinal muscular atrophy (SMA), trigeminal neuralgia or Werdnig-Hoffmann disease, and the method expresses the target gene in a motor neuron.

In some embodiments, the subject has aromatic 1-amino acid decarboxylase (AADC) deficiency, Batten disease, late infantile neuronal ceroid lipofuscinosis or mucopolysaccharidoses, and the method expresses the target gene in a neuron.

In some embodiments, the subject has achromatopsia, age-related macular degeneration (AMD), choroideremia, glaucoma, Leber congenital amaurosis 2, Leber hereditary optic neuropathy (LHON), retinitis pigmentosa, Stargardt disease, Usher syndrome type 1B, X-linked retinoschisis or X-linked retinitis pigmentosa, and the method expresses the target gene in a retina cell.

In some embodiments, the polynucleotide or the virus is introduced into the subject by intra-arterial, intracameral, intracisternal, intradermal, intradiaphragmatic, intramuscular, intranasal, intraperitoneal, intrapleural, intraportal, intravenous, intravitreal, pancreatic intraductal, retro-orbital, subconjunctival, subcutaneous, subretinal or sub-Tenon administration.

In some embodiments, the polynucleotide or the virus is introduced into the subject by intrathecal injection. In certain embodiments, the intrathecal injection is an intrathecal lumbar injection.

In some embodiments, the expression of the target gene occurs in the subject for at least about 1 month, for example, at least about: 2, 3, 4, 5, 6, 7, 8, 9, 10, 11 or 12 months. In some embodiments, the expression of the target gene occurs in the subject for at least 6 months.

Unless otherwise defined, all terms of art, notations and other scientific terms or terminology used herein are intended to have the meanings commonly understood by those of skill in the art to which this disclosure pertains. In some cases, terms with commonly understood meanings are defined herein for clarity and/or for ready reference, and the inclusion of such definitions herein should not necessarily be construed to represent a substantial difference over what is generally understood in the art. It will be further understood that terms, such as those defined in commonly used dictionaries, should be interpreted as having a meaning that is consistent with their meaning in the context of the relevant art and/or as otherwise defined herein.

The terminology used herein is for the purpose of describing particular embodiments only and is not intended to be limiting.

As used herein, the indefinite articles “a,” “an” and “the” should be understood to include plural reference unless the context clearly indicates otherwise.

Throughout this specification and the claims which follow, unless the context requires otherwise, the word “comprise,” and variations such as “comprises” and “comprising”, will be understood to imply the inclusion of, e.g., a stated integer or step or group of integers or steps, but not the exclusion of any other integer or step or group of integer or step. When used herein, the term “comprising” can be substituted with the term “containing” or “including.”

As used herein, “consisting of” excludes any element, step, or ingredient not specified in the claim element. When used herein, “consisting essentially of” does not exclude materials or steps that do not materially affect the basic and novel characteristics of the claim. Any of the terms “comprising,” “containing,” “including,” and “having,” whenever used herein in the context of an aspect or embodiment of the disclosure, can in some embodiments, be replaced with the term “consisting of,” or “consisting essentially of” to vary scopes of the disclosure.

As used herein, the conjunctive term “and/or” between multiple recited elements is understood as encompassing both individual and combined options. For instance, where two elements are conjoined by “and/or,” a first option refers to the applicability of the first element without the second. A second option refers to the applicability of the second element without the first. A third option refers to the applicability of the first and second elements together. Any one of these options is understood to fall within the meaning, and, therefore, satisfy the requirement of the term “and/or” as used herein. Concurrent applicability of more than one of the options is also understood to fall within the meaning, and, therefore, satisfy the requirement of the term “and/or.”

It should be understood that for all numerical bounds describing some parameter in this application, such as “about,” “at least,” “less than,” and “more than,” the description also necessarily encompasses any range bounded by the recited values. Accordingly, for example, the description “at least 1, 2, 3, 4, or 5” also describes, inter alia, the ranges 1-2, 1-3, 1-4, 1-5, 2-3, 2-4, 2-5, 3-4, 3-5, and 4-5, et cetera.

For all patents, applications, or other reference cited herein, such as non-patent literature and reference sequence information, it should be understood that they are incorporated by reference in their entirety for all purposes as well as for the proposition that is recited. Where any conflict exists between a document incorporated by reference and the present application, this application will control. All information associated with reference gene sequences disclosed in this application, such as GeneIDs or accession numbers (typically referencing NCBI accession numbers), including, for example, genomic loci, genomic sequences, functional annotations, allelic variants, and reference mRNA (including, e.g., exon boundaries or response elements) and protein sequences (such as conserved domain structures), as well as chemical references (e.g., PubChem compound, PubChem substance, or PubChem Bioassay entries, including the annotations therein, such as structures and assays, et cetera), are hereby incorporated by reference in their entirety.

Headings used in this application are for convenience only and do not affect the interpretation of this application.

Preferred features of each of the aspects provided by the disclosure are applicable to all of the other aspects of the disclosure mutatis mutandis and, without limitation, are exemplified by the dependent claims and also encompass combinations and permutations of individual features (e.g., elements, including numerical ranges and exemplary embodiments) of particular embodiments and aspects of the disclosure, including the working examples. For example, particular experimental parameters exemplified in the working examples can be adapted for use in the claimed disclosure piecemeal without departing from the disclosure. For example, for materials that are disclosed, while specific reference of each of the various individual and collective combinations and permutations of these compounds may not be explicitly disclosed, each is specifically contemplated and described herein. Thus, if a class of elements A, B, and C are disclosed as well as a class of elements D, E, and F and an example of a combination of elements A-D is disclosed, then, even if each is not individually recited, each is individually and collectively contemplated. Thus, in this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-groups of A-E, B-F, and C-E are specifically contemplated and should be considered disclosed from disclosure of A, B, and C; D, E, and F; and the example combination A-D. This concept applies to all aspects of this application, including elements of a composition of matter and steps of method of making or using the compositions.

The forgoing aspects of the disclosure, as recognized by the person having ordinary skill in the art following the teachings of the specification, can be claimed in any combination or permutation to the extent that they are novel and non-obvious over the prior art-thus, to the extent an element is described in one or more references known to the person having ordinary skill in the art, they may be excluded from the claimed disclosure by, inter alia, a negative proviso or disclaimer of the feature or combination of features.

### EXEMPLIFICATION

Translational neuroscience in recent years has brought new technologies closer to patients with the most common disabling neurological conditions. AAV gene therapy has emerged as a promising alternative strategy for translational medicine (Tuszynski M H et al. 2016). Recombinant adeno-associated viral vectors (AAV) are used as gene therapy vectors for treating inherited or acquired diseases (e.g., CNS diseases) and as essential research tools (e.g., in neuroscience), given their safety, serotype-dependent broad tropism and transduction efficiency (Aschauer, Kreuz et al. 2013; Samulski and Muzyczka 2014; Mak, Rajapaksha et al. 2017). AAV gene transfer into the central nervous system (CNS), especially neurons, has been widely used for mapping neural circuits in vivo and in vitro due to their high transfection efficiency and stable transgene expression (Hui Y et al. 2022).

However, one of the major barriers in targeted gene transfer is the specificity of transgene expression only in the cell types of interest. Despite their safety and broad tropism, important issues need to be addressed such as a limited payload capacity and a lack of small transcriptional control elements providing long-term, pan-neuronal transgene expression (e.g., in the CNS).

The use of a cell type-specific promoter may improve targeting and avoid transgene expression in antigen-presenting cells, abrogating unwanted immune responses against desired transgenes. Most neuron specific promoters are, in general, relatively weak in driving transgene expression. Commonly used gene promoters are relatively large and can be repressed a few months after viral transduction, risking long-term performance of gene therapy applications.

Recent improvements to recombinant AAVs, including capsid engineering and novel gene promoters to optimize transgene expression have substantially improved gene therapy applications (Mak, Rajapaksha et al. 2017; Hudry and Vandenberghe 2019; Ogden, Kelsic et al. 2019). AAV-9 variant PHP.eB (Chan, Jang et al. 2017), with an enhanced ability to permeate the mouse blood-brain barrier (BBB) and broadly transduce neurons both in the brain and spinal cord after peripheral vascular administration, is one example of recent capsid improvements (Dayton, Grames et al. 2018). A major limitation of recombinant AAVs is their small capsid with limited payload capacity of only ˜ 4.9 kb (Russell and Hirata 1998). Accordingly, the discovery of short transcriptional control element sequences that sustain strong and long-lived transcription is paramount to expand the transgene payload and achieve chronic therapeutic effect, particularly with one viral dose.

To offset this, high doses of AAV vectors are used, with the consequent induction of virus-associated toxicity and a strong host immune response to viral vectors. Several strategies have been used for targeted transgene expression in neural cells include engineering native cellular promoters, using recombinant transcriptional activators to achieve transcriptional amplification, and constructing expression cassettes by combining viral regulatory elements and a native cellular promoter.

Tissue specific promoters provide the advantage of limiting the expression to the desired cell or tissue. However, low levels of expression and/or large promoter size may limit their use. This could be a critical aspect for gene therapy where one or several therapeutic genes have to be expressed in cell-type specific fashion and for the lifespan of the patient/host.

For nervous system promoters, the pan-neuronal promoter neuron-specific enolase (NSE) drives stronger expression than ubiquitous promoters (Xu et al., 2001); however, its size of 2.2 kb limits its use in smaller vectors such as AAV. The sequences of the present disclosure have smaller size when compared to similar promoters such as EF1a, CAG, TH, CaMKIIa among others. This is an important advantage in the context of AAV vectors that have a limited genetic payload of 4.7 kb.

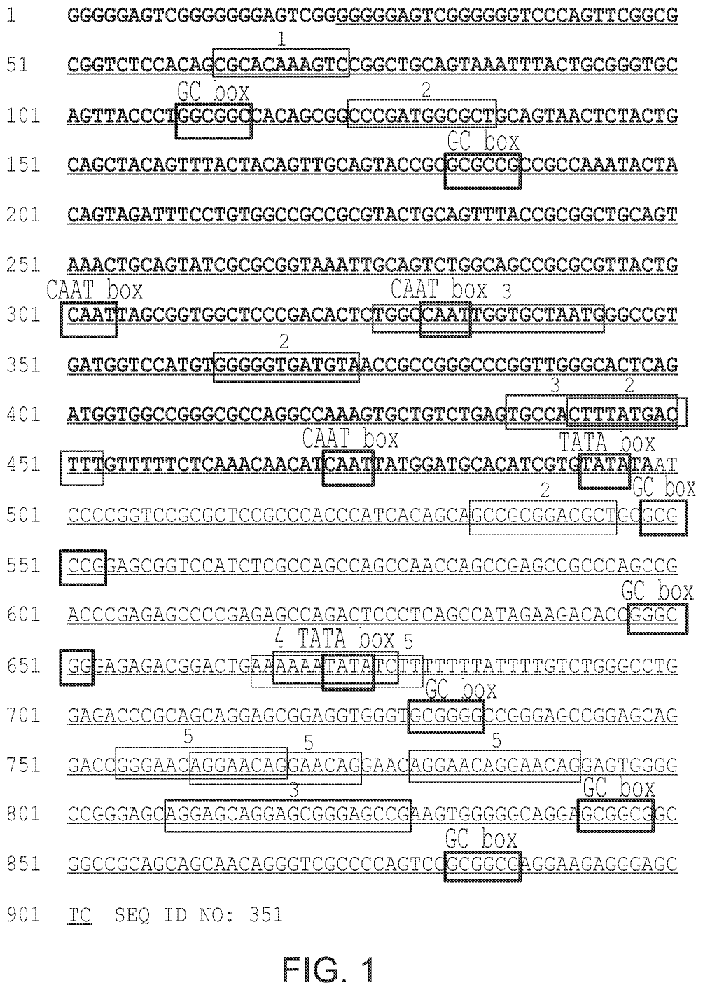

The LAP region of PRV encompasses two independent promoters, LAP1 and LAP2 (Cheung 1989; Cheung and Smith 1999; Jin and Scherba 1999; Jin, Schnitzlein et al. 2000) (FIG. 1). PRV LAP1 contain two GC boxes and three CAAT boxes upstream of the first TATA box. PRV LAP2 containing two GC boxes before the second TATA box (Cheung 1989; Jin and Scherba 1999; Taharaguchi, Kobayashi et al. 2002). It has been proposed that the binding of different transcription factor (TFs) to consensus promoter elements present in LAP, may facilitate escape from nucleosome silencing during the latent infection during the latent infection (Deshmane and Fraser 1989; Leib, Bogard et al. 1989; Devi-Rao, Goodart et al. 1991; Jin, Schnitzlein et al. 2000; Ono, Tomioka et al. 2007). In transgenic mouse lines, PRV LAP promoted transcription is neuron-specific in the absence of PRV infection (Taharaguchi, Yoshino et al. 2003). However, in transient expression assays, PRV LAP1 and LAP2 promote transcription both in cultured neuronal as well as non-neuronal cells (Cheung and Smith 1999; Taharaguchi, Kobayashi et al. 2002). Furthermore, the activity of tandem LAP1 and LAP2 sequences is significantly increased compared to LAP1 or LAP2 alone (Cheung and Smith 1999).

Given that the neural cell-specific promoters available to CNS clinical trial are either too large, and/or induce relatively weak gene expression, the LAP2 promoter architecture was modified in the studies described herein to improve the binding affinity of transcription factors and disrupt the binding of repressors, thus affecting promoter strength. A short, potent, and persistent promoter obtained from the genome of the herpesvirus pseudorabies virus (PRV) called alphaherpesvirus latency-associated promoter (LAP) was used. PRV LAP contains a cluster of two tandem promoter sequences, LAP1 and LAP2 that chronically drive transcription of latency-associated transcripts (LATs). Three small gene promoters were isolated from the genome of the alphaherpesvirus pseudorabies virus (PRV), called LAP1, LAP2, and LAP 1_2 (described in PCT Application No. PCT/US2020/016787, which is incorporated by reference in its entirety). These promoters show efficient and long-term transgene expression in the mouse CNS after a single viral vector administration.

The LAP2 promoter was originally isolated from the genome of herpesviruses where they chronically drive transcription of the latency-associated transcripts (LAT), even under highly repressible and adverse conditions. It was previously demonstrated that AAV-LAP2 is as a pan-neuronal promoter, with a potent and persistent transgene expression profile in the brain and spinal cord (Maturana C J, et al. 2020). Other commonly used promoters for expression in brain tissue such as the synapsin promoter, has been shown to decay or get repressed only after 5 months (Jackson et al., 2016). It has already been established that LAP2 can drive stable expression for 6 months in vivo and expect that expression is persistent for the lifespan of the host/patient.

The native nucleotide sequence of LAP2 (404 bp) was reduced to a minimal sequence of 232 bp for deletion of the first 160 bp upstream of the transcription start site (TATA box). To maintain the neural cell-specific transgene expression of native promoter, the transcription factor binds to CRE (CAMP response element) and CREB of 12 bp were conserved. Additionally, the promoter strength, using an enhancer element (modified CCCTC-binding factor, CTCF of 34 bp) positioned upstream of the minimized promoter, was increased. The new engineered promoter of 278 bp total size in combination with AAV-PHP.eB showed specific and potent transgene expression in the neuronal cells of the mouse brain after intravenous administration. In spinal cord and liver, a very low expression level was observed, while in kidney and skeletal muscle the transgene expression was not detected. This finding suggests a new approach to enhance gene expression through the combined incorporation of selected regulatory elements into an expression cassette. In addition, this MiniPromoter (SEQ ID NO: 349) improves the payload capacity of AAV vectors, expanding treatments for brain disorders.

### Example 1. Materials and Methods

Construction and Production of AAVs. AAV plasmids were packaged into AAV-PHP.eB as previously described in Maturana C J, et al. “Small Alphaherpesvirus Latency-Associated Promoters Drive Efficient and Long-Term Transgene Expression in the CNS” published in Mol. Ther. Methods Clin. Dev. 2020; 17:843-57 (the teachings of which are herein incorporated by reference in their entirety). The AAV titer was measured by qPCR using TaqMan (Thermo Fisher Scientific, Rockford, IL, USA) and reported as genome copies (gc)/ml (described in Chan A, et al. Optimized formulation buffer preserves Adeno-associated virus-9 infectivity after 4 degrees C. storage and freeze/thawing cycling. J Virol Methods. 2022:114598 and Maturana C J, et al. Single-Cell Quantification of Triple-AAV Vector Genomes Coexpressed in Neurons. Curr Protoc. 2022; 2(5):e430; the teachings of which are herein incorporated in their entirety by reference). AAV vectors were produced by the PNI Viral Core Facility (Princeton Neuroscience Institute, Princeton University). AAV plasmids contain mCherry driven by the LAP2 or MiniLAP2 promoter and include WPRE (woodchuck hepatitis virus post-transcriptional regulatory element) and SV40 polyA signal (simian virus 40 polyadenylation).

Animals. Timed-pregnancy Sprague-Dawley rats (Rattus norvegicus) were obtained from Hilltop Labs Inc. (Scottsdale, PA, USA). Embryonic day 17 [E17] rat embryos were harvested for isolation of superior cervical ganglia (SCG).

Wild type C57BL/6 male mice (Jackson Laboratories, Bar Harbor, ME) were used at five weeks of age. The mice were housed in 12-hr light/dark cycle with access to food and water ad libitum. All animal procedures were approved by the Institutional Animal Care and Use Committee of Princeton University (protocols 1947-19). Intravenous administration of rAAV vectors was performed via injection into the retro-orbital sinus. Mice each received 5×10¹¹ gc (in 100 ul) of a PHP.eB-LAP2-mCherry, PHP.eB-miniLAP2-mCherry or formulation buffer as control. Animals were sacrificed 30 days post injection by an overdose of ketamine (400 mg/kg)/xylazine (50 mg/kg) intraperitoneally and perfused with 4% paraformaldehyde (Fisher Scientific, Waltham, MA).

Primary Superior Cervical Ganglia Cell Culture. SCGs were dissociated with trypsin (2.5 mg/mL, Sigma-Aldrich, The Woodlands, TX, USA) and plated on poly-O-ornithine and laminin-coated dishes with media containing neurobasal media supplemented with 2% B-27, 100 ng/mL nerve growth factor (NGF), and 1% penicillin-streptomycin-glutamine (Thermo Fisher Scientific, Rockford, IL, USA). Three days after seeding, culture medium was treated with 0.1 mM cytosine-D-arabinofuranoside (Ara C) (Sigma-Aldrich, The Woodlands, TX, USA) for at least 2 days to eliminate dividing, non-neuronal cells. Neurons were grown at 37° C., 5% CO₂ for 10 days prior to AAV transduction with 3×10¹¹ AAV vg/dish.

Histology. Brain and spinal cord were post-fixed for 2 h at RT and liver, kidney, and quadriceps for 24 h at 4° C. Then all tissues were dehydrated in sucrose gradient as previously described in Laval K, et al. Mouse Footpad Inoculation Model to Study Viral-Induced Neuroinflammatory Responses. J Vis Exp. 2020 (160) and Maturana C J, et al. The Alphaherpesvirus Latency Associated Promoter 2 (LAP2) Drives Strong Transgene Expression in Peripheral Tissue Depending on Administration Route and AAV Serotype. bioRxiv. 2022:2022.08.04.502832 (the entire teachings of which are herein incorporated by reference). The tissues were sent to Histowiz Inc. (Brooklyn, NY) for immunohistochemistry (IHC) and in situ Hybridization (ISH). Paraffin block preparation and sectioning at 5 μm thickness for ISH and frozen block at 10 μm for IHC and immunofluorescence (IF) staining were ordered.

IHC and IF Staining. For chromogenic IHC analysis of mCherry the tissues were counterstained with hematoxylin. All the staining were performed using the Leica Bond RX automated Stainer (Leica Microsystems, Histowiz Inc). IF staining of brain and spinal cord sections was performed as previously described in Maturana C J, et al. Small Alphaherpesvirus Latency-Associated Promoters Drive Efficient and Long-Term Transgene Expression in the CNS. Mol Ther Methods Clin Dev. 2020; 17:843-57 (the entirety of which is herein incorporated by reference). Cryosections were blocked with 3% bovine serum albumin (BSA), 2% donkey serum, and 0.5% Triton X-100 (Sigma-Aldrich, St. Louis, MO) for 1 h at RT. The sections were incubated with primary antibodies overnight at 4° C. and further incubated with appropriate secondary antibodies for 1 h at RT. Cell nuclei were counterstained with DAPI (Thermo Fisher Scientific) and mounted using Vectashield Vibrance antifade mounting media (Molecular Probes, Eugene, OR).

RNA/DNA ISH. mCherry RNA ISH was performed using RNAscope 2.5 High Definition (HD) Red assay (Advanced Cell Diagnostics [ACD], Newark, CA, USA). Briefly, brain sections were boiled in the target retrieval solution for 15 min and pretreated with proteinase plus for 30 min at 40° C. The RNAscope target probes was incubated for 2 h at 40° C. and counterstained with hematoxylin. To detect mCherry DNA ISH, the protocol of RNA ISH was adapted according to the recommendations of the manufacturer. The chromogenic detection of DNA was performance using a RNase A treatment (5 mg/ml, QIAGEN, Hilden, Germany) for 30 min at 40° C. right before the target probe hybridization. The DNAscope target probes were incubated overnight at 40° C. and counterstained with hematoxylin.

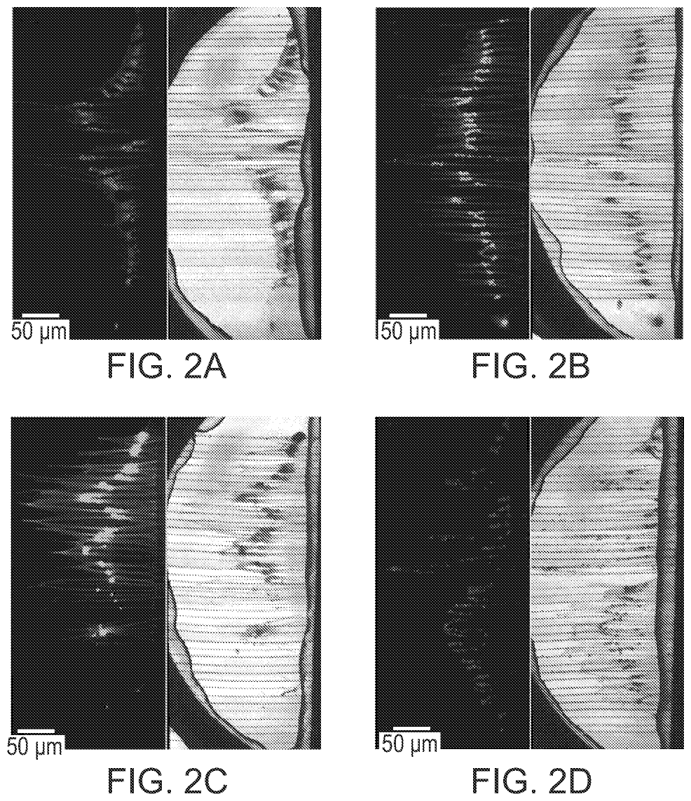

Imaging. Five days post infection, neurons were fixed and imaged in a Nikon Ti-E inverted epifluorescence microscope (Nikon Instruments, Tokyo, Japan), with a CoolSNAP ES2 camera (Photometrics, Tucson, AZ, USA) and the Nikon NIS-Elements software. Images (FIGS. 2A-2D) were captured using a 4× magnification objective.

Whole-slide scanning (40×) was performed on an Aperio AT2 (Leica Biosystems, Wetzlar, Germany) and histology image morphometry was performed using HALO software (Histowiz Inc). Fluorescent images were captured on a Leica SP8-LSCM confocal microscope (20×) and whole-brain were imaged with a NanoZoomer S600 microscope scanner (Hamamatsu, Hamamatsu, Japan). Image reconstructions of z-stacks and intensity projection images were generated in ImageJ software. Image quantifications were analyzed using QuPath 0.3.0 software (Maturana C J, et al. The Alphaherpesvirus Latency Associated Promoter 2 (LAP2) Drives Strong Transgene Expression in Peripheral Tissue Depending on Administration Route and AAV Serotype. bioRxiv. 2022:2022.08.04.502832; the entirety of which is herein incorporated by reference). The fluorescence intensity was calculated by adapting the corrected total cell fluorescence formula (Maturana C J, et al. High glucocorticoid levels during gestation activate the inflammasome in hippocampal oligodendrocytes of the offspring. Dev Neurobiol. 2017; 77(5):625-42; the entirety of which is herein incorporated by reference).

Statistical Analysis. Two-tailed Student's t test was used to compare between two groups. To compare among multiple groups, one-way ANOVA was used followed by Tukey's multiple comparisons test. A p statistically significant. Data are represented as the mean with SEM. Statistical analysis was performed with the GraphPad Prism 9.0 software (GraphPad, La Jolla, CA, USA).

### Example 2. Modified LAP2 Transcriptional Control Element Sequences

Using several computational tools such as EnhancerAtlas 2.0 and TRANSFAC 2.0 databases, JASPAR, CTCFBSDB 2.0 and Neural Network Promoter Prediction software, the LAP2 sequence was modified and optimized to arrive at the sequences referenced in the Table. These sequences are shorter than the original LAP2 sequence. Having transcription factor binding sites and enhancer sequences to restrict LAP2 transgene expression to certain cell types or tissues, these sequences are expected to maintain similar transcription activity as shown in vitro in primary neurons.

TABLE
Cell-/Tissue-specific Enhancer and Transcriptional
Control Element Sequences

| | | SEQ ID | |
|---|---|---|---|
| | | | Transcriptional |
| | | Enhancer | Control |
| Cell Type | Tissue | Elements | Elements |
| myoblast | gastrocnemius | NOs: 5-22 | NOs: 177-194 |
| | muscle | | |
| whole pancreas | pancreas | NOs: 23-43 | NOs: 195-215 |
| cells | | | |
| whole lung cells | lung | NOs: 44-52 | NOs: 216-224 |
| whole retina cells | neonate retina | NOs: 53-90 | NOs: 225-262 |
| microglia | brain/spinal cord | NOs: 91-108 | NOs: 263-280 |
| astrocyte | brain/spinal cord | NOs: 109-113 | NOs: 281-285 |
| neuron | nucleus accumbens | NOs: 114-126 | NOs: 286-298 |
| | (forebrain) | | |
| neuron | cerebral cortex | NOs: 127-160 | NOs: 299-332 |
| | (brain) | | |
| glutamatergic | brain/spinal cord | NO: 161 or 162 | NO: 333 or 334 |
| neuron | | | |
| GABAergic | brain/spinal cord | NO: 163 or 164 | NO: 335 or 336 |
| neuron | | | |
| dopaminergic | brain/spinal cord | NOs: 165-169 | NOs: 337-341 |
| neuron | | | |
| cholinergic | brain/spinal cord | NO: 170 | NO: 342 |
| neuron | | | |
| motor neuron | brain/spinal cord | NO: 171 or 172 | NO: 343 or 344 |
| neuron | brain/spinal cord | NOs: 173-176, | NOs: 345-349 |
| | | 350 | |

These novel variants of LAP2 transcriptional control elements are expected to drive cell-type or tissue-specific expression to levels similar or higher than that of full length LAP2 in vivo. The sequences could be used to express therapeutic transgenes in several organs and tissues of animal models and humans. Following systemic administration of viral or non-viral vectors, tissue-specific transcriptional control elements can be used to restrict expression to liver, muscle, pancreas, lung, retina, or brain/spinal cord. These sequences can potentially improve gene therapy and research such as neuroscience research to dissect brain connectivity and function in diverse types such as cell-glutamatergic, GABAergic, dopaminergic, cholinergic, and motor neurons, astrocytes, and microglia. Further, by reducing the size of LAP2, there will be additional payload in the AAV capsid that can be devoted to deliver larger or multiple therapeutic transgenes. The AAV capsid can package up to 4,700 bp and the use of smaller promoters is critical for certain diseases requiring delivery of large payloads. Finally, these sequences can be used in gene therapy applications besides central nervous system in diseases affecting peripheral organs.

### Example 3. Transcriptional Activity in Primary Superior Cervical Ganglion (SCG) Neurons

The sequences of the present disclosure are shorter than the original LAP2 sequence but maintain similar transcriptional activity in primary SCG neurons. The addition of exogenous transcription factor binding sites and enhancers to LAP2, can increase transgene expression levels by the formation of transcription regulatory complexes, as shown for EnhancerLAP2.169 (SEQ ID NO:345) and EnhancerLAP2.170 (SEQ ID NO:346). It is expected that these novel sequences can provide cell-type or tissue-specific expression to levels similar or higher than that of full length LAP2 in vivo.

Four AAV recombinants were packaged into serotype PHP.eB capsids by standard methods, each containing LAP2 full length (SEQ ID NO:4; 404 bp), EnhancerLAP2.169 (SEQ ID NO:345; 260 bp), EnhancerLAP2.170 (SEQ ID NO:346; 258 bp), or EnhancerLAP2.171 (SEQ ID NO:347; 278 bp) promoter sequences. LAP2 full length was used as a positive transgene expression control. AAV-PHP.eB, an engineered capsid variant, derived from serotype 2/9, was selected for this study. To verify the in vitro performance of each promoter, primary SCG neuronal cultures were transduced with 3×10¹¹ viral genomes (vg) of each AAV.

All four AAV recombinants expressed the fluorescent reporter mCherry (FIGS. 2A-2D). Both EnhancerLAP2.169 (SEQ ID NO:345) and EnhancerLAP2.170 (SEQ ID NO: 346) drove strong transgene expression, all the SCG neurons in the dish showed intense fluorescent reporter expression (FIGS. 2B and 2C). EnhancerLAP2.171 (SEQ ID NO:347), however provided weak expression (FIG. 2D) when compared to LAP2 full length (FIG. 2A).

In addition to AAV, it is believed that LAP promoters can be used in other types of gene expression vectors, such as plasmid, BAC, cosmid or other non-viral or viral system to express therapeutic transgene(s). AAV serotype PHP.eB was selected because this AAV can cross the blood-brain barrier with high affinity and systemically infect the whole brain, allowing for a broader validation of vectors.

It is envisioned that the promoter sequences of the present disclosure will be useful when strong, long-term transgene expression with tissues/cell-type specificity is needed in vitro, ex vivo or in vivo in animals and humans, and may be especially useful in situations where there is payload constrains but long-lasting promoter transcriptional activity is needed. AAV's are the gold standard for human gene therapy with FDA approved AAV vectors already in use in the clinic. It is expected that LAP2 variants to will work well when used in other viral vectors such as lentiviruses, retroviruses, and adenoviruses and also in non-viral gene delivery modalities.

Gene promoters are essential regulatory elements to achieve stable gene expression. For certain diseases, therapeutic gene expression has to be stable, long-lasting and targeted to specific organs and even cell-types within the that organ. Because of the important size restriction of AAV vectors, such promoters need to be as short as possible to allow expression or large or multiple therapeutic genes. The novel sequences provided herein are less prone to repression or inactivation than most commonly used promoters, making them particularly useful for gene therapies requiring chronic expression of the therapeutic transgene after one single vector administration.

### Example 4: MiniLAP2

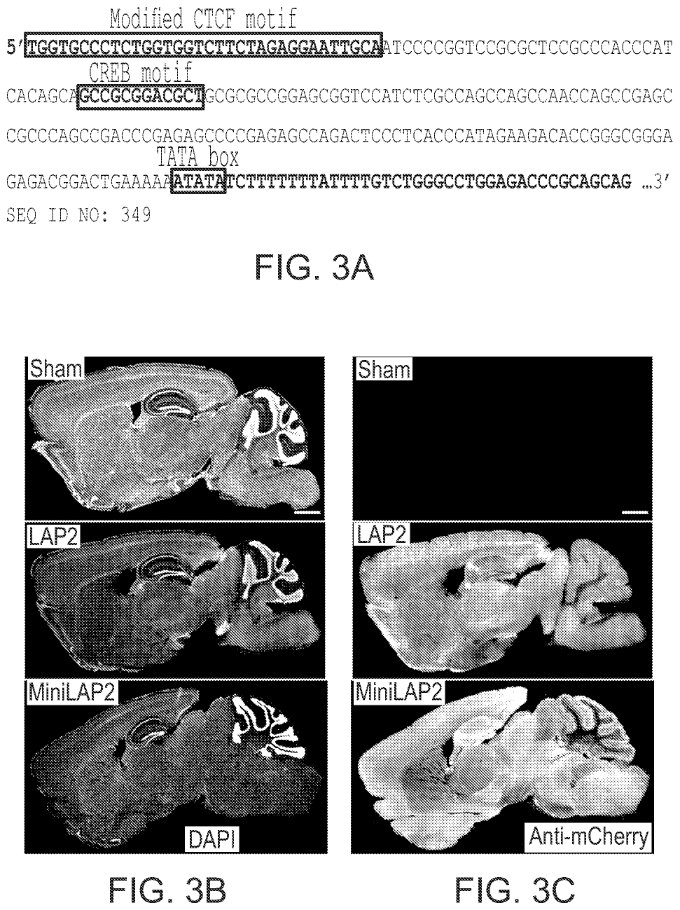

To generate the MiniLAP2 promoter, the LAP2 sequence was modified upstream or at the 5′ end of the transcription start site (TATA box). The first 160 base pairs (bp) were removed and only the transcription factor binds to CRE (CREB) was preserved. The modified CTCF motif was added upstream of the CREB site. FIG. 3A.

The LAP2 (404 bp) and MiniLAP2 (278 bp) promoter was packaged into AAV-PHP.eB vector driving transcription of the mCherry fluorescent reporter. Expression cassette includes WPRE (woodchuck hepatitis virus post-transcriptional regulatory element) and SV40 polyA (simian virus 40 polyadenylation) polyadenylation site. Each AAV vector was administered either by unilateral intravenous administration into retro-orbital sinus at a dose 5×10¹¹ vg per C57BL/6 mice. Sham-negative control was injected with formulation buffer. Brain, spinal cord, liver, kidney, and quadriceps was collected at 30 days post AAV administration for analysis.

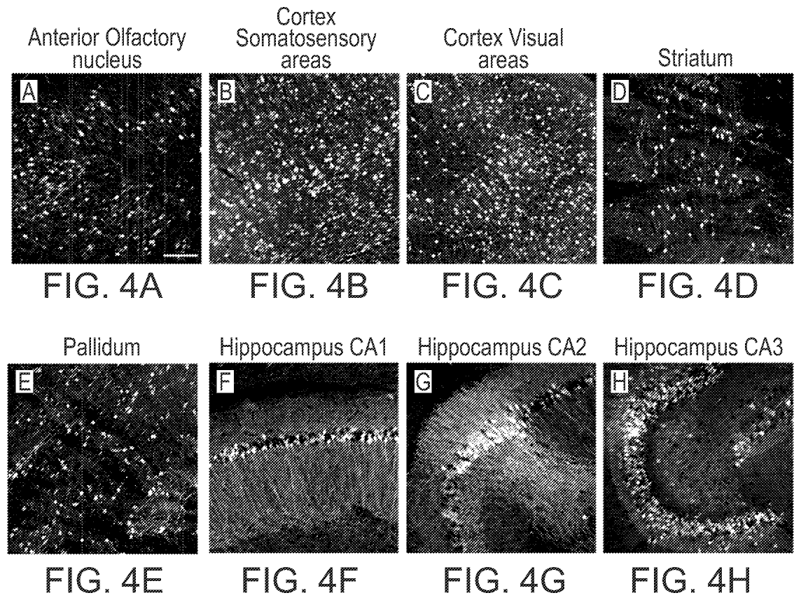

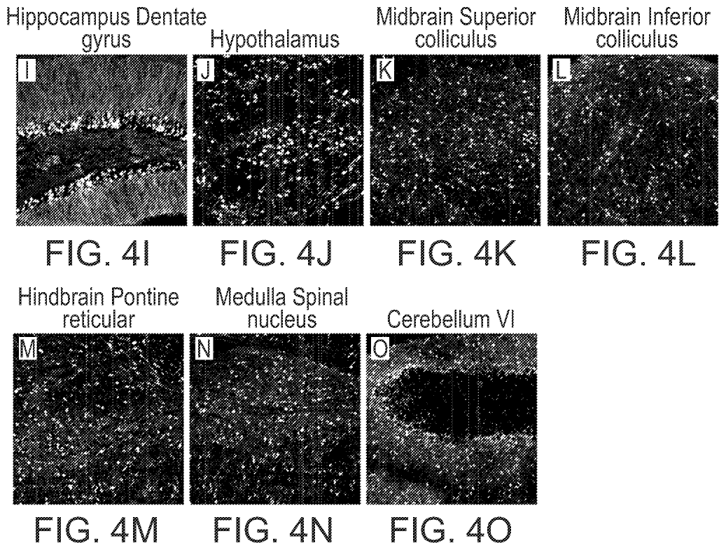

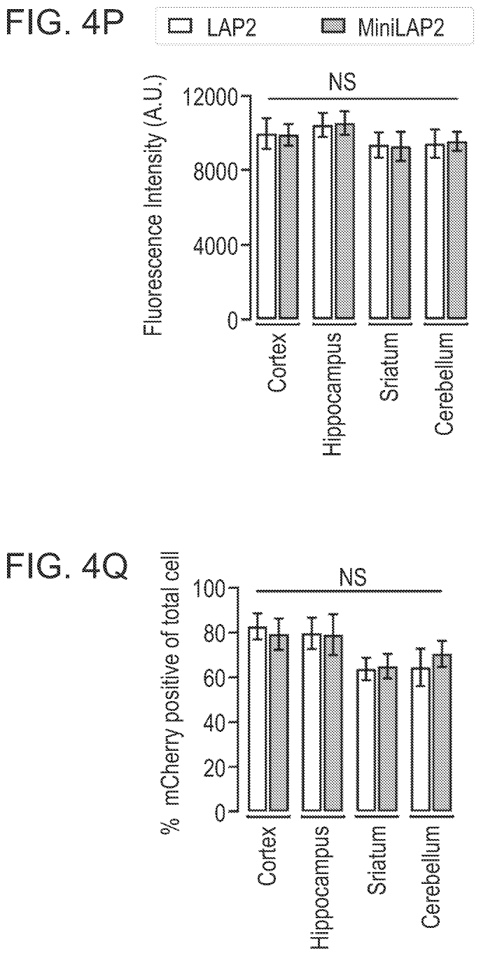

Sagittal sections showing whole-brain distribution were analyzed (FIG. 3B and FIG. 3C). Next, confocal images show that MiniLAP2-mCherry drives widespread and potent expression throughout the mouse brain (FIGS. 4A-4Q).

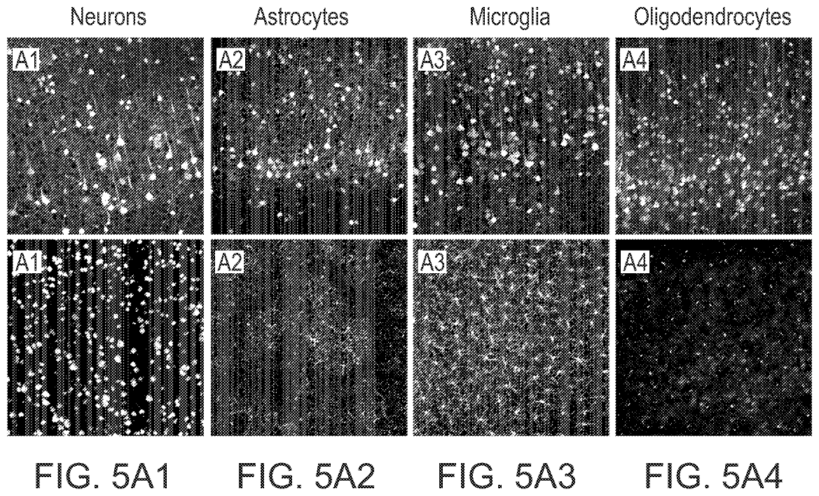

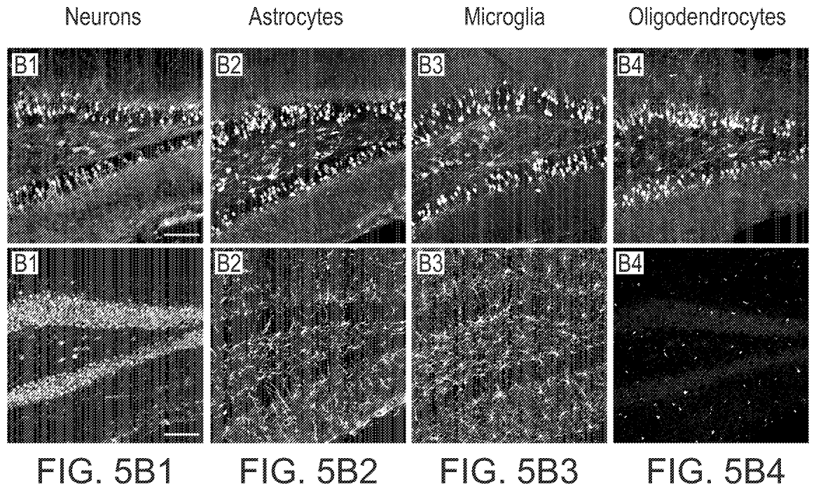

To determine AAV-MiniLAP2 transgene expression, sagittal brain sections were taken from mice and immunostained with anti-mCherry or a nueronal marker in the cortex (FIGS. 5A1-5A4) and hippocampus (FIGS. 5B1-5B4). FIGS. 5A1 and 5B1 show localization using anti-mCherry antibody and the pan-neuronal marker NeuN. FIGS. 5A2 and 5B2 show localization in astrocytes using anti-mCherry antibody and the astrocytic marker GFAP. FIGS. 5A3 and 5B3 show localization in microglia using anti-mCherry antibody and the microglia marker Iba1. FIGS. 5A4 and 5B4 show localization in oligodendrocytes using anti-mCherry antibody and the oligodendrocyte marker Olig2. The NeuN, GFAP, and Iba1 signal can localize with the cell nucleus as well as the cytoplasm, while the staining for the Olig2 signal is mostly nuclear. Quantification of these data show the percentage of neurons expressing transgene in cortex (75%) and hippocampus (72%), astrocytes in cortex (2%) and hippocampus (3%), microglia in cortex (1%) and hippocampus (1%), and oligodendrocyte in cortex (2%) and hippocampus (2%). Three tissue sections were analyzed per animal.

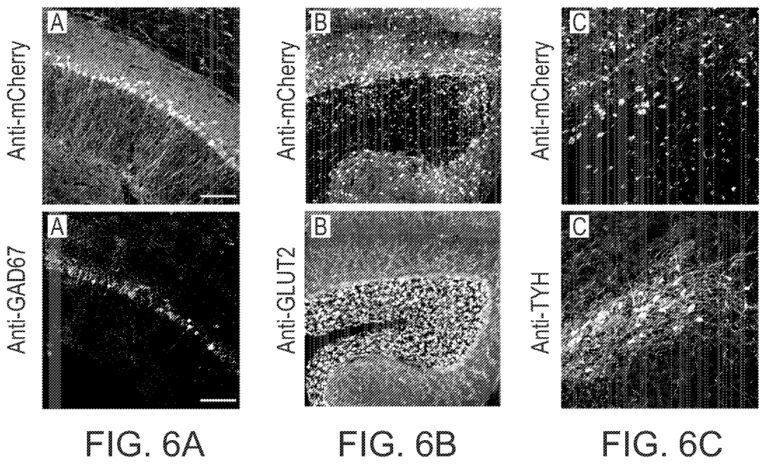

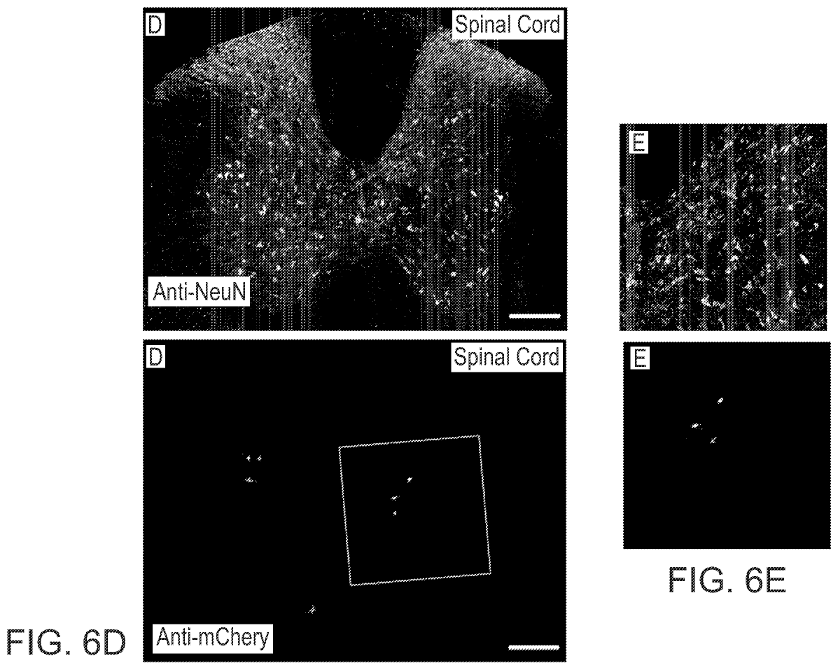

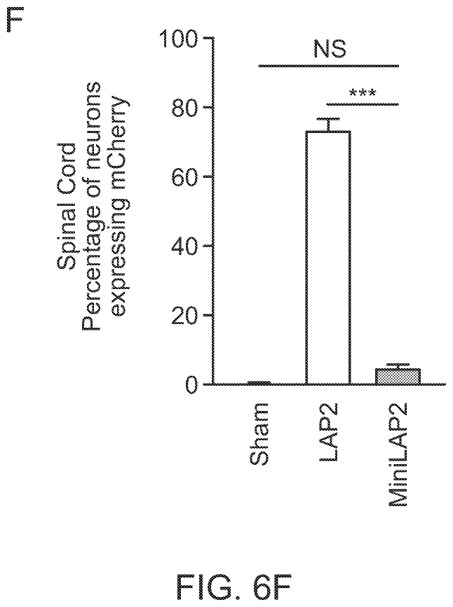

Further experimentation shows that MiniLAP2 drives pan-neuronal transgene expression after systemic AAV-PHP.eB administration. Using confocal microcopy in combination with immunostaining, sagittal sections of brain tissue were analyzed (FIGS. 6A-6E). GABAergic neurons in the hippocampus CA1 were stained using the glutamic acid decarboxylase 67 marker (GAD67) (FIG. 6A). Glutamatergic neurons in the cerebellum VIII were stained using the vesicular glutamate transporter 2 marker (vGLUT2) (FIG. 6B). Dopaminergic neurons in the substantia nigra were stained using the tyrosine hydroxylase marker (TYH) (FIG. 6C). Next, confocal images of cross lumbar spinal cord sections were analyzed using anti-mCherry antibody and the pan-neuronal marker NeuN (FIGS. 6D and 6E). Higher magnification of the dorsal horn shows minimal transgene expression (FIG. 6E).

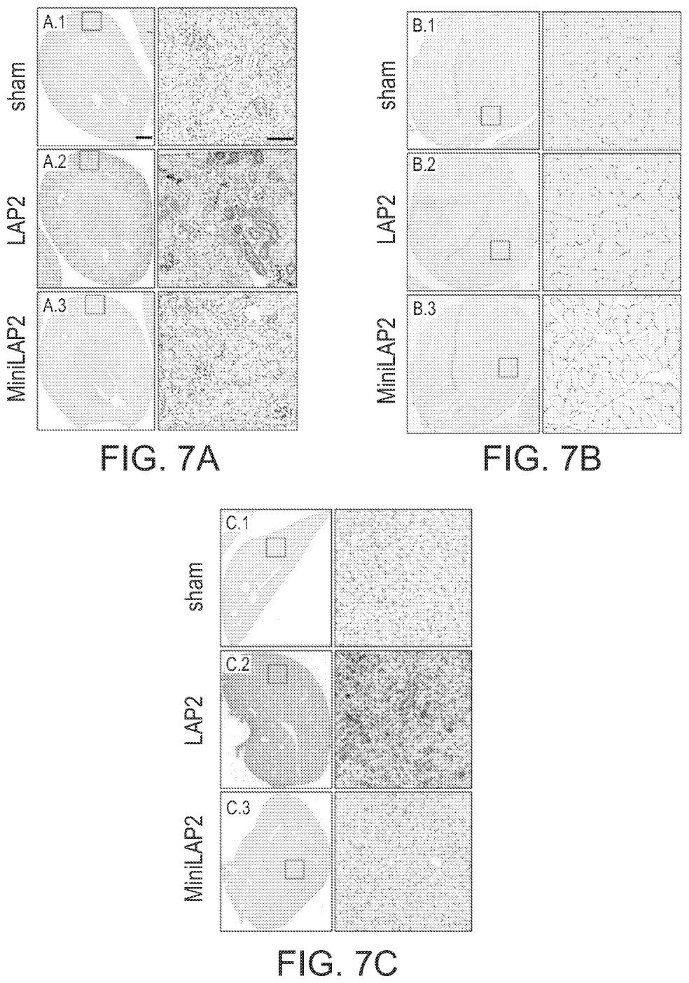

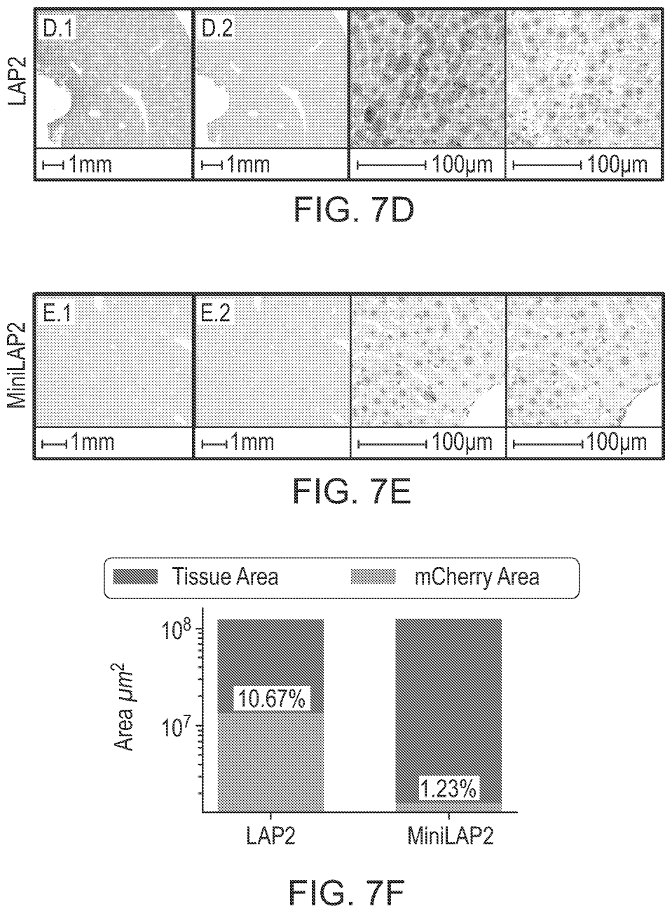

Systemic administration of PHP.eB-MiniLAP2 leads decreased liver targeting and negative enrichment in the kidney and muscle. Immunohistochemical staining was used to compare sham treatment (negative control) with PHP.eB-LAP2 and PHP.eB-MiniLAP2 treatment of various tissue types (FIGS. 7A-7E). While AAV-LAP2 shows targeting in kidney, muscle, and liver tissues, the absence of signal in kidney and muscle from AAV-MiniLAP2 was noted. Quantification of positively stained nuclei per area analyzed confirms this observation where AAV-LAP2 has 10.67% and AAV-MiniLAP2 only 1.23% (FIG. 7F).

The teachings of all patents, published applications and references cited herein are incorporated by reference in their entirety.

While example embodiments have been particularly shown and described, it will be understood by those skilled in the art that various changes in form and details may be made therein without departing from the scope of the embodiments encompassed by the appended claims.

What is claimed is:

1. A polynucleotide comprising a transcriptional control element, wherein the transcriptional control element comprises a promoter sequence having at least 80% sequence identity to SEQ ID NO:1, SEQ ID NO:2, SEQ ID NO:3 or SEQ ID NO:4 and a cell- or tissue-specific enhancer element.

2. The polynucleotide of claim 1, wherein the enhancer element comprises a nucleotide sequence having at least 90% sequence identity to any one of SEQ ID NOs: 5-176 or SEQ ID NO: 350.

3. The polynucleotide of claim 2, wherein the enhancer element comprises a nucleotide sequence identical to any one of SEQ ID NOs: 5-176 or SEQ ID NO: 350.

4. The polynucleotide of claim 2, wherein the enhancer element comprises a nucleotide sequence having at least 90% sequence identity to any one of SEQ ID NOs: 5-22, SEQ ID NOs: 23-43, SEQ ID NOs: 44-52, SEQ ID NOs: 53-90, SEQ ID NOs: 91-108, SEQ ID NOs: 109-113, SEQ ID NOs: 114-126, SEQ ID NOs: 127-160, SEQ ID NO:161 or SEQ ID NO:162, SEQ ID NO:163 or SEQ ID NO:164, SEQ ID NOs: 165-169, SEQ ID NO: 170, SEQ ID NO:171 or SEQ ID NO: 172, or SEQ ID NOs: 173-176 or SEQ ID NO: 350.

5. The polynucleotide of claim 1, wherein the transcriptional control element comprises a nucleotide sequence having at least 80% sequence identity to any one of SEQ ID NOs: 177-349.

6. The polynucleotide of claim 5, wherein the transcriptional control element comprises a nucleotide sequence that is identical to any one of SEQ ID NOs: 177-349.

7. The polynucleotide of claim 5, wherein the transcriptional control element comprises a nucleotide sequence having at least 80% sequence identity to any one of SEQ ID NOs: 177-194, SEQ ID NOs: 195-215, SEQ ID NOs: 216-224, SEQ ID NOs: 225-262, SEQ ID NOs: 263-280, SEQ ID NOs: 281-285, SEQ ID NOs: 286-298, SEQ ID NOs: 299-332, SEQ ID NO:333 or SEQ ID NO:334, SEQ ID NO:335 or SEQ ID NO: 336, SEQ ID NOs: 337-341, SEQ ID NO:342, SEQ ID NO:343 or SEQ ID NO: 344, or SEQ ID NOs: 345-349.

8. The polynucleotide of any one of claims 1-7, further comprising a cloning site for insertion of a coding sequence for a target gene.

9. The polynucleotide of any one of claims 1-7, further comprising a coding sequence for a target gene operably linked to the transcriptional control element.

10. The polynucleotide of claim 9, wherein the coding sequence for the target gene comprises at least 4 kilobases.

11. The polynucleotide of any one of claims 1-10, further comprising an inducer element.

12. The polynucleotide of any one of claims 1-11, further comprising a selectable marker element.

13. The polynucleotide of any one of claims 1-12, wherein the polynucleotide is a vector.

14. The polynucleotide of claim 13, wherein the vector is a gene therapy vector.

15. The polynucleotide of claim 13 or 14, wherein the vector is a viral vector.

16. The polynucleotide of claim 15, wherein the viral vector is an adeno-associated virus (AAV) vector.

17. The polynucleotide of claim 13 or 14, wherein the vector is a non-viral vector.

18. A virus comprising the polynucleotide of any one of claims 1-16.

19. The virus of claim 18, wherein the virus is an adeno-associated virus (AAV).

20. A host cell comprising the polynucleotide of any one of claims 1-17 or the virus of claim 18 or 19.

21. The host cell of claim 20, wherein the host cell is a mammalian cell.

22. The host cell of claim 20 or 21, wherein the host cell is an in vitro cell or an ex vivo cell.

23. The host cell of claim 20 or 21, wherein the host cell is a cell of a subject.

24. The host cell of any one of claims 20-23, wherein the host cell is a myoblast, a pancreatic cell, a lung cell, a neonatal retina cell, a microglia cell, an astrocyte or a neuron.

25. The host cell of claim 24, wherein the neuron is a neuron of the nucleus accumbens, a neuron of the cerebral cortex, a glutamatergic neuron, a GABAergic neuron, a dopaminergic neuron, a cholinergic neuron, or a motor neuron.

26. The host cell of any one of claims 20-25, wherein the enhancer element comprises a nucleotide sequence having at least 90% sequence identity to any one of SEQ ID NOs: 5-176 or SEQ ID NO: 350.

27. The host cell of claim 26, wherein the enhancer element comprises a nucleotide sequence identical to any one of SEQ ID NOs: 5-176 or SEQ ID NO: 350.

28. The host cell of claim 26, wherein:
the host cell is a myoblast of gastrocnemius muscle, and the enhancer element comprises a nucleotide sequence having at least 90% sequence identity to any one of SEQ ID NOs: 5-22;
the host cell is a pancreatic cell, and the enhancer element comprises a nucleotide sequence having at least 90% sequence identity to any one of SEQ ID NOs: 23-43;
the host cell is a lung cell, and the enhancer element comprises a nucleotide sequence having at least 90% sequence identity to any one of SEQ ID NOs: 44-52;
the host cell is a neonatal retina cell, and the enhancer element comprises a nucleotide sequence having at least 90% sequence identity to any one of SEQ ID NOs: 53-90;
the host cell is a microglia cell, and the enhancer element comprises a nucleotide sequence having at least 90% sequence identity to any one of SEQ ID NOs: 91-108;
the host cell is an astrocyte, and the enhancer element comprises a nucleotide sequence having at least 90% sequence identity to any one of SEQ ID NOs: 109-113;
the host cell is a neuron of the nucleus accumbens, and the enhancer element comprises a nucleotide sequence having at least 90% sequence identity to any one of SEQ ID NOs: 114-126;
the host cell is a neuron of the cerebral cortex, and the enhancer element comprises a nucleotide sequence having at least 90% sequence identity to any one of SEQ ID NOs: 127-160;
the host cell is a glutamatergic neuron, and the enhancer element comprises a nucleotide sequence having at least 90% sequence identity to SEQ ID NO:161 or SEQ ID NO: 162;
the host cell is a GABAergic neuron, and the enhancer element comprises a nucleotide sequence having at least 90% sequence identity to SEQ ID NO:163 or SEQ ID NO: 164;
the host cell is a dopaminergic neuron, and the enhancer element comprises a nucleotide sequence having at least 90% sequence identity to any one of SEQ ID NOs: 165-169;
the host cell is a cholinergic neuron, and the enhancer element comprises a nucleotide sequence having at least 90% sequence identity to SEQ ID NO:170;
the host cell is a motor neuron, and the enhancer element comprises a nucleotide sequence having at least 90% sequence identity to SEQ ID NO:171 or SEQ ID NO: 172; or
the host cell is a neuron, and the enhancer element comprises a nucleotide sequence having at least 90% sequence identity to any one of SEQ ID NOs: 173-176 or SEQ ID NO: 350.

29. The host cell of any one of claims 20-25, wherein the transcriptional control element comprises a nucleotide sequence having at least 80% sequence identity to any one of SEQ ID NOs: 177-349.

30. The host cell of claim 29, wherein the transcriptional control element comprises a nucleotide sequence identical to any one of SEQ ID NOs: 177-349.

31. The host cell of claim 29, wherein:
the host cell is a myoblast of gastrocnemius muscle, and the transcriptional control element comprises a nucleotide sequence having at least 80% sequence identity to any one of SEQ ID NOs: 177-194;
the host cell is a pancreatic cell, and the transcriptional control element comprises a nucleotide sequence having at least 80% sequence identity to any one of SEQ ID NOs: 195-215;
the host cell is a lung cell, and the transcriptional control element comprises a nucleotide sequence having at least 80% sequence identity to any one of SEQ ID NOs: 216-224;
the host cell is a neonatal retina cell, and the transcriptional control element comprises a nucleotide sequence having at least 80% sequence identity to any one of SEQ ID NOs: 225-262;
the host cell is a microglia cell, and the transcriptional control element comprises a nucleotide sequence having at least 80% sequence identity to any one of SEQ ID NOs: 263-280;
the host cell is an astrocyte, and the transcriptional control element comprises a nucleotide sequence having at least 80% sequence identity to any one of SEQ ID NOs: 281-285;
the host cell is a neuron of the nucleus accumbens, and the transcriptional control element comprises a nucleotide sequence having at least 80% sequence identity to any one of SEQ ID NOs: 286-298;
the host cell is a neuron of the cerebral cortex, and the transcriptional control element comprises a nucleotide sequence having at least 80% sequence identity to any one of SEQ ID NOs: 299-332;
the host cell is a glutamatergic neuron, and the transcriptional control element comprises a nucleotide sequence having at least 80% sequence identity to SEQ ID NO: 333 or SEQ ID NO:334;
the host cell is a GABAergic neuron, and the transcriptional control element comprises a nucleotide sequence having at least 80% sequence identity to SEQ ID NO: 335 or SEQ ID NO:336;
the host cell is a dopaminergic neuron, and the transcriptional control element comprises a nucleotide sequence having at least 80% sequence identity to any one of SEQ ID NOs: 337-341;
the host cell is a cholinergic neuron, and the transcriptional control element comprises a nucleotide sequence having at least 80% sequence identity to SEQ ID NO: 342;
the host cell is a motor neuron, and the transcriptional control element comprises a nucleotide sequence having at least 80% sequence identity to SEQ ID NO: 343 or SEQ ID NO:344; or
the host cell is a neuron, and the transcriptional control element comprises a nucleotide sequence having at least 80% sequence identity to any one of SEQ ID NOs: 345-349.

32. A pharmaceutical composition, comprising the polynucleotide of any one of claims 1-17, the virus of claim 18 or 19, or the host cell of any one of claims 20-31, and one or more pharmaceutically acceptable excipients, diluents, or carriers.

33. A method of expressing a target gene in a host cell, comprising contacting the host cell with the polynucleotide of any one of claims 9-17 or the virus of claim 18 or 19 under conditions whereby the polynucleotide or the virus is introduced into the host cell, and expression of the target gene occurs in the host cell.

34. The method of claim 33, wherein the host cell is a mammalian cell.

35. The method of claim 33 or 34, wherein the host cell is an in vitro cell or an ex vivo cell.

36. The method of any one of claims 33-35, wherein the host cell is a cell of a subject.

37. The method of claim 36, wherein the cell of the subject is allogeneic.

38. The method of claim 36, wherein the cell of the subject is autologous or syngeneic.

39. The method of any one of claims 33-38, wherein the polynucleotide is introduced into the host cell by transfection or infection.

40. A method of expressing a target gene in a subject in need thereof, comprising administering to the subject an effective amount of the polynucleotide of any one of claims 9-17, the virus of claim 18 or 19, or the pharmaceutical composition of claim 32, whereby expression of the target gene occurs in a host cell of the subject.

41. The method of any one of claims 33-40, wherein the host cell is a myoblast, a pancreatic cell, a lung cell, a neonatal retina cell, a microglia cell, an astrocyte or a neuron.

42. The method of claim 41, wherein the target gene is expressed in a neuron of the subject.

43. The method of claim 42, wherein the neuron is a neuron of the nucleus accumbens, a neuron of the cerebral cortex, a glutamatergic neuron, a GABAergic neuron, a dopaminergic neuron, a cholinergic neuron, or a motor neuron.

44. The method of any one of claims 36-43, wherein the subject is a human.

45. The method of any one of claims 36-44, wherein the subject has a condition selected from the group consisting of: Alzheimer's disease (AD), amyotrophic lateral sclerosis (ALS), ataxia telangietacsia, cancer, Dopa-responsive dystonia, a fatty liver disease, Fragile X syndrome, Friedreich ataxia, Giant axonal neuropathy, glycogen storage disease type 2 (Pompe), Huntington's disease (HD), Krabbe disease, multiple sclerosis (MS), multiple system atrophy (MSA), neuronal ceroid lipofuscinosis (Batten disease) Niemann-Pick disease A, B and C, pain, Parkinson's disease (PD), Pelizaeus-Merzbacher disease, primary lateral sclerosis, a prion disease, proximal myotonic myopathy, spinal muscular atrophy (SMA), Spinocerebellar ataxia type 1, 2 and 3, Tay-Says disease, and X-linked adrenoleukodystrophy.

46. The method of any one of claims 36-45, wherein the polynucleotide or the virus is introduced into the subject by intra-arterial, intracameral, intracisternal, intradermal, intradiaphragmatic, intramuscular, intranasal, intraperitoneal, intrapleural, intraportal, intrathecal, intravenous, intravitreal, pancreatic intraductal, retro-orbital, subconjunctival, subcutaneous, subretinal or sub-Tenon administration.

47. The method of claim 46, wherein the polynucleotide or the virus is introduced into the subject by intrathecal injection.

48. The method of claim 47, wherein the intrathecal injection is an intrathecal lumbar injection.

49. The method of any one of claims 36-48, wherein the expression of the target gene occurs in the subject for at least 6 months.


SEQUENCE LISTING (351 sequences)

SEQ ID NO: 1 - DNA, 232 bp, Pseudorabies virus
  source 1..232: mol_type = genomic DNA
atatatctttttttattttgtctgggcctggagacccgcagcaggagcggaggtgggtgcggggccgggagccggagcaggaccgggaacaggaacaggaacaggaacaggaacaggaacaggagtggggccgggagcaggagcaggagcgggagccgaagtgggggcaggagcggcggcggccgcagcagcaacagggtcgccccagtccgcggcgaggaagagggagctc

SEQ ID NO: 2 - DNA, 347 bp, synthetic construct
  misc_feature 1..347: note = Synthetic Promoter
  source 1..347: mol_type = other DNA
cacccatcacagcagccgcggacgctgcgcgccggagcggtccatctcgccagccagccaaccagccgagccgcccagccgacccgagagccccgagagccagactccctcagccatagaagacaccgggcgggagagacggactgaaaaaatatatctttttttattttgtctgggcctggagacccgcagcaggagcggaggtgggtgcggggccgggagccggagcaggaccgggaacaggaacaggaacaggaacaggaacaggaacaggagtggggccgggagcaggagcaggagcgggagccgaagtgggggcaggagcggcggcggccgcagcagcaaca

SEQ ID NO: 3 - DNA, 383 bp, synthetic construct
  misc_feature 1..383: note = Synthetic Promoter
  source 1..383: mol_type = other DNA
cacccatcacagcagccgcggacgctgcgcgccggagcggtccatctcgccagccagccaaccagccgagccgcccagccgacccgagagccccgagagccagactccctcagccatagaagacaccgggcgggagagacggactgaaaaaatatatctttttttattttgtctgggcctggagacccgcagcaggagcggaggtgggtgcggggccgggagccggagcaggaccgggaacaggaacaggaacaggaacaggaacaggaacaggagtggggccgggagcaggagcaggagcgggagccgaagtgggggcaggagcggcggcggccgcagcagcaacagggtcgccccagtccgcggcgaggaagagggagctc

SEQ ID NO: 4 - DNA, 404 bp, Pseudorabies virus
  source 1..404: mol_type = genomic DNA
atccccggtccgcgctccgcccacccatcacagcagccgcggacgctgcgcgccggagcggtccatctcgccagccagccaaccagccgagccgcccagccgacccgagagccccgagagccagactccctcagccatagaagacaccgggcgggagagacggactgaaaaaatatatctttttttattttgtctgggcctggagacccgcagcaggagcggaggtgggtgcggggccgggagccggagcaggaccgggaacaggaacaggaacaggaacaggaacaggaacaggagtggggccgggagcaggagcaggagcgggagccgaagtgggggcaggagcggcggcggccgcagcagcaacagggtcgccccagtccgcggcgaggaagagggagctc

SEQ ID NO: 5 - DNA, 80 bp, synthetic construct
  misc_feature 1..80: note = EnhancerLAP2.1
  source 1..80: mol_type = other DNA
cccaggaactcaggcttaccaggaaccccaatatttaacataagggtagcaactcagtaggaatactaagaatccaaatg

SEQ ID NO: 6 - DNA, 100 bp, synthetic construct
  misc_feature 1..100: note = EnhancerLAP2.2
  source 1..100: mol_type = other DNA
tgtctcctgaaaagggaagcactgagtgactgactgcagggctttcagcccctcccttagccccaggagtgggtgctcctttctagacatcagatcccct

SEQ ID NO: 7 - DNA, 100 bp, synthetic construct
  misc_feature 1..100: note = EnhancerLAP2.3
  source 1..100: mol_type = other DNA
gaggtgtggccttgggagggtggagtaggggcagtgtgtgaagagggaagggcaggacttggtctcctgttccctcccctgattcccagtgtctttgcct

SEQ ID NO: 8 - DNA, 80 bp, synthetic construct
  misc_feature 1..80: note = EnhancerLAP2.4
  source 1..80: mol_type = other DNA
agttaaaagaaaaaagaaaagacagcaagaaatcaaccttggtatcaagtgagaaactctaaggctgatgagcctcttac

SEQ ID NO: 9 - DNA, 80 bp, synthetic construct
  misc_feature 1..80: note = EnhancerLAP2.5
  source 1..80: mol_type = other DNA
gtaaggaagatgtcctttatggtatatttggccgtgttatttgtggctgcagagatggaggtgggcgttcacgtcgtgat

SEQ ID NO: 10 - DNA, 100 bp, synthetic construct
  misc_feature 1..100: note = EnhancerLAP2.6
  source 1..100: mol_type = other DNA
ctgaattccttcacctttgctacctttttctgctcttgctggctgatgcaactcagctgttctggctcagactcctctcctagctgactgattcaatctg

SEQ ID NO: 11 - DNA, 60 bp, synthetic construct
  misc_feature 1..60: note = EnhancerLAP2.7
  source 1..60: mol_type = other DNA
atgctgtaaagtagaagagataggatgagtttgattgttacagccaaccaacagaaatat

SEQ ID NO: 12 - DNA, 90 bp, synthetic construct
  misc_feature 1..90: note = EnhancerLAP2.8
  source 1..90: mol_type = other DNA
gggctggaaaataaaaagtggggttgttgtggttaggggacaattagggaagtgatgagggaggtgggaggagcgagggtggagagctca

SEQ ID NO: 13 - DNA, 80 bp, synthetic construct
  misc_feature 1..80: note = EnhancerLAP2.9
  source 1..80: mol_type = other DNA
tgggcaggtatgcatcacctttttagtggatgaggagagggagagccatgatggcacggtgccatgacagtgtcctctgt

SEQ ID NO: 14 - DNA, 100 bp, synthetic construct
  misc_feature 1..100: note = EnhancerLAP2.10
  source 1..100: mol_type = other DNA
accgttgctagaaggggaaggaaggaggcagaggacaggtgagctcaccccaggccagctcagaaagtaccacaccaactctgcccacctaactctcctt

SEQ ID NO: 15 - DNA, 60 bp, synthetic construct
  misc_feature 1..60: note = EnhancerLAP2.11
  source 1..60: mol_type = other DNA
aggtccctgagcagaaagggacagagagcaggtggagccagctctgtctccctctgtttt

SEQ ID NO: 16 - DNA, 80 bp, synthetic construct
  misc_feature 1..80: note = EnhancerLAP2.12
  source 1..80: mol_type = other DNA
ccctggggaacaaaatgctgtgtgaagctacacatctggtcattctctgagtccctctgtatgtaaccctggttgccacc

SEQ ID NO: 17 - DNA, 90 bp, synthetic construct
  misc_feature 1..90: note = EnhancerLAP2.13
  source 1..90: mol_type = other DNA
gtctcacctaaagggacaaaaaagaaatatgagcaaggagctagggttggagagagctagggttggacctggtaagaagctgaccctaga

SEQ ID NO: 18 - DNA, 80 bp, synthetic construct
  misc_feature 1..80: note = EnhancerLAP2.14
  source 1..80: mol_type = other DNA
ctggaggaggagaaatacaggctatgttgggtcatgccgtggggccatgagtaccccttggaatggtgggaggggtaccc

SEQ ID NO: 19 - DNA, 90 bp, synthetic construct
  misc_feature 1..90: note = EnhancerLAP2.15
  source 1..90: mol_type = other DNA
gtggaacccacagatatagagggtcaatgacaggtacactgtttaaaggtataacattacaaaacaaaacaaaactagcacactcataaa

SEQ ID NO: 20 - DNA, 70 bp, synthetic construct
  misc_feature 1..70: note = EnhancerLAP2.16
  source 1..70: mol_type = other DNA
atacttgggacttccagaatgctatccctaccctgcctgccaggcatcagtgagtatgatgacatttgga

SEQ ID NO: 21 - DNA, 70 bp, synthetic construct
  misc_feature 1..70: note = EnhancerLAP2.17
  source 1..70: mol_type = other DNA
cgccttccgccccgcccgcgccccgcctcgtccgcccgcgtcccggtctccgccgcacgcccgcagagtg

SEQ ID NO: 22 - DNA, 90 bp, synthetic construct
  misc_feature 1..90: note = EnhancerLAP2.18
  source 1..90: mol_type = other DNA
ccaggaggtgagggcagccaccccacagcccctgcatccctgtccccaccccatggcttttcccacactgacatcatgtccccgtctcct

SEQ ID NO: 23 - DNA, 70 bp, synthetic construct
  misc_feature 1..70: note = EnhancerLAP2.19
  source 1..70: mol_type = other DNA
gggtaactccgcggggctcggggcgggctggccgcgcgggtcggcgtcgctgcacctcagcgagcgcctg

SEQ ID NO: 24 - DNA, 100 bp, synthetic construct
  misc_feature 1..100: note = EnhancerLAP2.20
  source 1..100: mol_type = other DNA
tgcagcagtcccaagttcaagaaggttccaagagataaggagggcccagtgctatctctaggagccagtgccaaggtttgcagccaaaatcaccgagacc

SEQ ID NO: 25 - DNA, 80 bp, synthetic construct
  misc_feature 1..80: note = EnhancerLAP2.21
  source 1..80: mol_type = other DNA
ggcgcaagggtcccgggactggtcaccagaggggaacttagggtgaggagggcagcgctcctcggtgtggctctaactca

SEQ ID NO: 26 - DNA, 60 bp, synthetic construct
  misc_feature 1..60: note = EnhancerLAP2.22
  source 1..60: mol_type = other DNA
tatgaggtagcatctttggtttccttcatcttattactgagcgctggggccagagacctt

SEQ ID NO: 27 - DNA, 90 bp, synthetic construct
  misc_feature 1..90: note = EnhancerLAP2.23
  source 1..90: mol_type = other DNA
actaaatagggatggatatgcagggcggaggtaaggaccctgcagccaaactagcctcgttggactaagggggctacaatgtggtgtcta

SEQ ID NO: 28 - DNA, 70 bp, synthetic construct
  misc_feature 1..70: note = EnhancerLAP2.24
  source 1..70: mol_type = other DNA
ggtctgtggtgaccctgtccctctgtacatacctctgtcatatttcccccacccctccatgctatctgct

SEQ ID NO: 29 - DNA, 70 bp, synthetic construct
  misc_feature 1..70: note = EnhancerLAP2.25
  source 1..70: mol_type = other DNA
ccgggacaggtccgtgcggatcgggagaggccagcgcggcccgggctgaggtggatggtggggtcagggg

SEQ ID NO: 30 - DNA, 90 bp, synthetic construct
  misc_feature 1..90: note = EnhancerLAP2.26
  source 1..90: mol_type = other DNA
tctgtggcaaggggaggcgggcgcggaggaggtgagcaggcatggcagtgagcccctcctagccctgcctcccctcttccatccccacca

SEQ ID NO: 31 - DNA, 90 bp, synthetic construct
  misc_feature 1..90: note = EnhancerLAP2.27
  source 1..90: mol_type = other DNA
aggtctgtgtggggtgagtaggggtgaggtaagtggacactggggttgccttcccagggtccctggcagagtagccctctgttctcttac

SEQ ID NO: 32 - DNA, 100 bp, synthetic construct
  misc_feature 1..100: note = EnhancerLAP2.28
  source 1..100: mol_type = other DNA
gtgaggtaggttttccgccttacctgtcctgtgggggtggatggagcaggtctggggccaaggccgattgcagggcccatgcagcactcctctgggagct

SEQ ID NO: 33 - DNA, 60 bp, synthetic construct
  misc_feature 1..60: note = EnhancerLAP2.29
  source 1..60: mol_type = other DNA
gtgggtggggtcagggactttcttcaggaggttaaccttgttacacgtgaaattttcttc

SEQ ID NO: 34 - DNA, 90 bp, synthetic construct
  misc_feature 1..90: note = EnhancerLAP2.30
  source 1..90: mol_type = other DNA
acctgttgtggggagaacacagaaaggaggggtaagtcggatctcttggagatgcccaccccctgagcccacctaccccactggtgccat

SEQ ID NO: 35 - DNA, 80 bp, synthetic construct
  misc_feature 1..80: note = EnhancerLAP2.31
  source 1..80: mol_type = other DNA
ccacctggggacatgggcgagagggagggacaaggcaagctgaaaggaaggacagccaggggctggccctgggctgacct

SEQ ID NO: 36 - DNA, 90 bp, synthetic construct
  misc_feature 1..90: note = EnhancerLAP2.32
  source 1..90: mol_type = other DNA
acattctgaaaggttcaaaatagtgttaaagcagaagtatataatactttactgtcattaaatgcacacccaaggacagcaaagaatgtt

SEQ ID NO: 37 - DNA, 90 bp, synthetic construct; identical to SEQ ID NO: 36
  misc_feature 1..90: note = EnhancerLAP2.33
  source 1..90: mol_type = other DNA

SEQ ID NO: 38 - DNA, 100 bp, synthetic construct
  misc_feature 1..100: note = EnhancerLAP2.34
  source 1..100: mol_type = other DNA
ctcagcatttttttacctggctcaatagccggggactccacagaagcagagagagagagagagagagagagagagagagagagagagagagagagagacg

SEQ ID NO: 39 - DNA, 70 bp, synthetic construct
  misc_feature 1..70: note = EnhancerLAP2.35
  source 1..70: mol_type = other DNA
tctgtgggaaggcaggaagagttgaggctatgaccacagatagaaaacagcagggccacttagccccaac

SEQ ID NO: 40 - DNA, 100 bp, synthetic construct
  misc_feature 1..100: note = EnhancerLAP2.36
  source 1..100: mol_type = other DNA
tgaagcttgggtggagccacttcgcacaggttgggatgaggaccctgatttgtgtaggtcagggccaagctctcctggtgctcagctcacatggctcctt

SEQ ID NO: 41 - DNA, 90 bp, synthetic construct
  misc_feature 1..90: note = EnhancerLAP2.37
  source 1..90: mol_type = other DNA
cttccaggtgctgggggaaagatttcagacgcatttctgggtgtggctggaccagggcagggggtgggataggactggagggacagacgg

SEQ ID NO: 42 - DNA, 100 bp, synthetic construct
  misc_feature 1..100: note = EnhancerLAP2.38
  source 1..100: mol_type = other DNA
cttaaaaaaaaaaacccactatgttccatctgagaactaagcaaattattctctcctctgggtcctatatatagccacaggcagctacaatagacctata

SEQ ID NO: 43 - DNA, 80 bp, synthetic construct
  misc_feature 1..80: note = EnhancerLAP2.39
  source 1..80: mol_type = other DNA
ctatcagttaacttgtgcagctaccctgatgtgttctggagactctgtttagcgtatggccttttaaactgtctctttct

SEQ ID NO: 44 - DNA, 90 bp, synthetic construct
  misc_feature 1..90: note = EnhancerLAP2.40
  source 1..90: mol_type = other DNA
acagaagcctttccgggactgccgaggcttatctgaacttaagaatcgttggaatgttggattacttcagagtactctgttttttcattt

SEQ ID NO: 45 - DNA, 80 bp, synthetic construct
  misc_feature 1..80: note = EnhancerLAP2.41
  source 1..80: mol_type = other DNA
ccgtaagtgtcccgggggtgggcggggcgcgggccctgcaggctctggaggggacagcacgggagtggcccgcccctgga

SEQ ID NO: 46 - DNA, 100 bp, synthetic construct
  misc_feature 1..100: note = EnhancerLAP2.42
  source 1..100: mol_type = other DNA
catctttactgtgggaagaggaggcgttggatggctttaaagcggaccctgaatgcattcgatttttatttatttattgtaaggaaataaaattgaccta

SEQ ID NO: 47 - DNA, 60 bp, synthetic construct
  misc_feature 1..60: note = EnhancerLAP2.43
  source 1..60: mol_type = other DNA
agtcccgccgcgcgccccacggccccgcgggagacgacctcgcaggcaggccgccgcgca

SEQ ID NO: 48 - DNA, 100 bp, synthetic construct
  misc_feature 1..100: note = EnhancerLAP2.44
  source 1..100: mol_type = other DNA
taacacccaaggccccagttctgagtggctccttggattcccaggggcaatcaagcttcataccctacatcctcaagaatcctagttaggcacataactt

SEQ ID NO: 49 - DNA, 100 bp, synthetic construct
  misc_feature 1..100: note = EnhancerLAP2.45
  source 1..100: mol_type = other DNA
ggtaggtcccctccgcccttgagggcacgggcctcagccccggccccacccccgcggcccgtggccccggccccagctcgcagtcgagaggggtgacggg

SEQ ID NO: 50 - DNA, 90 bp, synthetic construct
  misc_feature 1..90: note = EnhancerLAP2.46
  source 1..90: mol_type = other DNA
atctgccctaaaaaaacaacctcgttgaggtacaggcgcaaggctgcacgccggtgacctgatgctgggcgaccagcgccttcccgccag

SEQ ID NO: 51 - DNA, 70 bp, synthetic construct
  misc_feature 1..70: note = EnhancerLAP2.47
  source 1..70: mol_type = other DNA
aaaggttctggcttgcgtttgcttttcaaatgacgtcacggaaaggcggagtaagccagtgcgcatgcac

SEQ ID NO: 52 - DNA, 100 bp, synthetic construct
  misc_feature 1..100: note = EnhancerLAP2.48
  source 1..100: mol_type = other DNA
agtgaggtgagttctgcgccctgggtgcctctgtcacagagttctgccgccgacgtcagtggcccgcagcttactgtctgtgctcttctctgtccccgca

SEQ ID NO: 53 - DNA, 80 bp, synthetic construct
  misc_feature 1..80: note = EnhancerLAP2.49
  source 1..80: mol_type = other DNA
cctggggagacaggagagacttgagactgttttccctccctgtgcccgctttctatccctccttccattccccgtggccc

SEQ ID NO: 54 - DNA, 60 bp, synthetic construct
  misc_feature 1..60: note = EnhancerLAP2.50
  source 1..60: mol_type = other DNA
tcctcttcacccggcaccgtgcgcccccccccccgcaacccccccaagacagcaccgccg

SEQ ID NO: 55 - DNA, 70 bp, synthetic construct
  misc_feature 1..70: note = EnhancerLAP2.51
  source 1..70: mol_type = other DNA
tggtgagtggcagaggcaggtgctgggcctcccaggggagtcagggtagaatccaccagcccttctccct

SEQ ID NO: 56 - DNA, 90 bp, synthetic construct
  misc_feature 1..90: note = EnhancerLAP2.52
  source 1..90: mol_type = other DNA
ggcctgcacctgcgcagaggggaagggagttgagtccggccgtgcgcttcggggtgcaccctgaaggtccccctcccccctgcacctttc

SEQ ID NO: 57 - DNA, 100 bp, synthetic construct
  misc_feature 1..100: note = EnhancerLAP2.53
  source 1..100: mol_type = other DNA
agcttgcaggttggtagactcggcattgggggaggggtacgggggtcgcagggacactgggggttcacagccaccccaaatccttaggcagggtgtgcag

SEQ ID NO: 58 - DNA, 100 bp, synthetic construct
  misc_feature 1..100: note = EnhancerLAP2.54
  source 1..100: mol_type = other DNA
atttggcctgcaatgggagatttaaattgtcagaagtgaggcaacagagatggcggctgggtccagcacacctctcccctttggggacctcggagtagcc

SEQ ID NO: 59 - DNA, 80 bp, synthetic construct
  misc_feature 1..80: note = EnhancerLAP2.55
  source 1..80: mol_type = other DNA
acactgcagcagagagcacagtgcatcaggcacacatgggacacaaagagccacccccaggccttgcatgccaccccgtg

SEQ ID NO: 60 - DNA, 90 bp, synthetic construct
  misc_feature 1..90: note = EnhancerLAP2.56
  source 1..90: mol_type = other DNA
tttgctgaaatagcaaggcaagtaagttcgctcagcaagtttcttactttcaatgtttttttgtttgcagtctatagatttttgtataaa

SEQ ID NO: 61 - DNA, 60 bp, synthetic construct
  misc_feature 1..60: note = EnhancerLAP2.57
  source 1..60: mol_type = other DNA
actaaagcaattaccccaaacggggggtggaacctgccacctccctctcccaggtgttta

SEQ ID NO: 62 - DNA, 100 bp, synthetic construct
  misc_feature 1..100: note = EnhancerLAP2.58
  source 1..100: mol_type = other DNA
ttttcccaagaaccacttaacccacgacaacggtgggtttccccttcaaaaggaagatttccactctcccatgatgcacaccaacccctgggcaattcta

SEQ ID NO: 63 - DNA, 80 bp, synthetic construct
  misc_feature 1..80: note = EnhancerLAP2.59
  source 1..80: mol_type = other DNA
ccagtgagctcttggggcggggtgagagtgcaggggtcttcaacagaatccagaaaccttggtccaagcccttttctctc

SEQ ID NO: 64 - DNA, 80 bp, synthetic construct
  misc_feature 1..80: note = EnhancerLAP2.60
  source 1..80: mol_type = other DNA
ctaaaaagaaacagaggccgtcaatggccggccacccaagggccagaaccagctctccagcccttccctaccgtgtgtcc

SEQ ID NO: 65 - DNA, 90 bp, synthetic construct
  misc_feature 1..90: note = EnhancerLAP2.61
  source 1..90: mol_type = other DNA
caaggtccgcggccccgggggaggagaagcgcctgctttctgcgcagtcgccccgcccggactggggggcgggacttaggcgacccgggg

SEQ ID NO: 66 - DNA, 100 bp, synthetic construct
  misc_feature 1..100: note = EnhancerLAP2.62
  source 1..100: mol_type = other DNA
gcccaatgtatgatggaggggcccaggcccatcgtggctctggatgcaagggggaggggtgggcccttcagatcactgttgaacacgagggatgttcttg

SEQ ID NO: 67 - DNA, 100 bp, synthetic construct; identical to SEQ ID NO: 66
  misc_feature 1..100: note = EnhancerLAP2.63
  source 1..100: mol_type = other DNA

SEQ ID NO: 68 - DNA, 100 bp, synthetic construct
  misc_feature 1..100: note = EnhancerLAP2.64
  source 1..100: mol_type = other DNA
gggcagcagcgaccctccccccaacacctttcccgtcccctcccccacggccggcactacctcccccccaccccaggaacaccccccccccgtgcctccc

SEQ ID NO: 69 - DNA, 100 bp, synthetic construct; identical to SEQ ID NO: 68
  misc_feature 1..100: note = EnhancerLAP2.65
  source 1..100: mol_type = other DNA

SEQ ID NO: 70 - DNA, 90 bp, synthetic construct
  misc_feature 1..90: note = EnhancerLAP2.66
  source 1..90: mol_type = other DNA
cggtgagcgcgctgcaccctcccgggaacccgcgcgcccgtccccgtcccggagctagttgtaatgctttttcctccaaaccctgttttt

SEQ ID NO: 71 - DNA, 80 bp, synthetic construct
  misc_feature 1..80: note = EnhancerLAP2.67
  source 1..80: mol_type = other DNA
gtagggcccaaccgcacgggcctgggactttgtcgttggggctgagagcccaggccaaattctcatctcccctttccccg

SEQ ID NO: 72 - DNA, 80 bp, synthetic construct
  misc_feature 1..80: note = EnhancerLAP2.68
  source 1..80: mol_type = other DNA
cacccacactctgcatgctgcatgccacaaatgtgatgtgtgaagaaaaaaagaacagaacgcaagcagaaacgcaaaca

SEQ ID NO: 73 - DNA, 90 bp, synthetic construct
  misc_feature 1..90: note = EnhancerLAP2.69
  source 1..90: mol_type = other DNA
ctagaaacaccaaagaaacgcttgaatgtcaggaaatgaggcacaacacaaaatgactcgtcacgcccaaggggaggcaaggtcccggct

SEQ ID NO: 74 - DNA, 100 bp, synthetic construct
  misc_feature 1..100: note = EnhancerLAP2.70
  source 1..100: mol_type = other DNA
catgacgccctgcgagagaaaaggccgcggtcagactcgccccacccaccgcgcgacccgggcccagagagaacacaagggaggccactcctctcaacta

SEQ ID NO: 75 - DNA, 90 bp, synthetic construct
  misc_feature 1..90: note = EnhancerLAP2.71
  source 1..90: mol_type = other DNA
ggctgggtgagtgctcccccctttctcccgcagtgctttgcgcgcggccggtgggcgggggagggaggggaggggaggcggcggcggcag

SEQ ID NO: 76 - DNA, 100 bp, synthetic construct
  misc_feature 1..100: note = EnhancerLAP2.72
  source 1..100: mol_type = other DNA
gaaggtaccaaactggccttggtctaaggtctctggactgagggctgtgtgagcacatgcctcagggaagcttggggcactgctgagtgacctggattcc

SEQ ID NO: 77 - DNA, 80 bp, synthetic construct
  misc_feature 1..80: note = EnhancerLAP2.73
  source 1..80: mol_type = other DNA
ccatggcaggtaggatggtccgcgcaggcggggacggggcagcgggacgcgacctctgcagccagttgctatttcgctgc

SEQ ID NO: 78 - DNA, 80 bp, synthetic construct; identical to SEQ ID NO: 45
  misc_feature 1..80: note = EnhancerLAP2.74
  source 1..80: mol_type = other DNA

SEQ ID NO: 79 - DNA, 100 bp, synthetic construct
  misc_feature 1..100: note = EnhancerLAP2.75
  source 1..100: mol_type = other DNA
ctgtggaacaaaatggggaggttggcgacccagcagaggtccccacccacagctgtggggcagaccgctcctcactgtgaaatgggcaagggagccttga

SEQ ID NO: 80 - DNA, 90 bp, synthetic construct
  misc_feature 1..90: note = EnhancerLAP2.76
  source 1..90: mol_type = other DNA
gtgaggcttagagggccatctccccatccatgggtctttctgtgattttcgatggatttgggagcctcaactgagatgcctgtctcccta

SEQ ID NO: 81 - DNA, 70 bp, synthetic construct
  misc_feature 1..70: note = EnhancerLAP2.77
  source 1..70: mol_type = other DNA
cctgcttggactccagtggcccaattcacttcatccaggcactgcaacccagagaggaaggcagggagac

SEQ ID NO: 82 - DNA, 60 bp, synthetic construct
  misc_feature 1..60: note = EnhancerLAP2.78
  source 1..60: mol_type = other DNA
ggaaggtaaatcctggcctgggcgcggggcccaccaggactgtcctgcccgcccgtgccc

SEQ ID NO: 83 - DNA, 90 bp, synthetic construct
  misc_feature 1..90: note = EnhancerLAP2.79
  source 1..90: mol_type = other DNA
gtaccaggcaagaaaacctaccgatttttgatcatatgcctggggtactttactcagtagctctggcctctgcttttctttctctgcctt

SEQ ID NO: 84 - DNA, 80 bp, synthetic construct; identical to SEQ ID NO: 35
  misc_feature 1..80: note = EnhancerLAP2.80
  source 1..80: mol_type = other DNA

SEQ ID NO: 85 - DNA, 100 bp, synthetic construct
  misc_feature 1..100: note = EnhancerLAP2.81
  source 1..100: mol_type = other DNA
gcagtccctgtatcagagaagggaaaaagaattgagtcagctggtgagagctgttatctagcatcccaggactccccagaatcccccacgtgttcactca

SEQ ID NO: 86 - DNA, 70 bp, synthetic construct
  misc_feature 1..70: note = EnhancerLAP2.82
  source 1..70: mol_type = other DNA
gccagcacctgcggagaggcggggagtgagcggacaccggatggcgccccagccctcccgcccgcggccc

SEQ ID NO: 87 - DNA, 80 bp, synthetic construct
  misc_feature 1..80: note = EnhancerLAP2.83
  source 1..80: mol_type = other DNA
gcctaagaaggaagaggacgcagctcaagcggcgtggagtacagcggcacaccgggtagcagctccacagcctaccccat

SEQ ID NO: 88 - DNA, 80 bp, synthetic construct
  misc_feature 1..80: note = EnhancerLAP2.84
  source 1..80: mol_type = other DNA
ggctgtcgcgttaccccggtaactacgagaggcgcacttcccggaagtgtgggcgcaccacgcggtctcccgcccctcca

SEQ ID NO: 89 - DNA, 80 bp, synthetic construct
  misc_feature 1..80: note = EnhancerLAP2.85
  source 1..80: mol_type = other DNA
cccctccccgacccccgcgtagttccgtctctcgttcgtagctcaaaccgaggttacgcgcaggcgcgccctgcccgctc

SEQ ID NO: 90 - DNA, 70 bp, synthetic construct; identical to SEQ ID NO: 51
  misc_feature 1..70: note = EnhancerLAP2.86
  source 1..70: mol_type = other DNA

SEQ ID NO: 91 - DNA, 80 bp, synthetic construct; identical to SEQ ID NO: 53
  misc_feature 1..80: note = EnhancerLAP2.87
  source 1..80: mol_type = other DNA

SEQ ID NO: 92 - DNA, 80 bp, synthetic construct; identical to SEQ ID NO: 45
  misc_feature 1..80: note = EnhancerLAP2.88
  source 1..80: mol_type = other DNA

SEQ ID NO: 93 - DNA, 90 bp, synthetic construct
  misc_feature 1..90: note = EnhancerLAP2.89
  source 1..90: mol_type = other DNA
ggcaggtgagcggtgcgctccgggcggggccggcccggcgggggcggggacgcgggcacgggccggtcccggtgacctctgagccgcttc

SEQ ID NO: 94 - DNA, 90 bp, synthetic construct; identical to SEQ ID NO: 75
  misc_feature 1..90: note = EnhancerLAP2.90
  source 1..90: mol_type = other DNA

SEQ ID NO: 95 - DNA, 80 bp, synthetic construct
  misc_feature 1..80: note = EnhancerLAP2.91
  source 1..80: mol_type = other DNA
ggtatttgcatctgagagcaatttgtcacacggcgattcgtctgctgggtgggggaggggcggggcggggggcccggtcg

SEQ ID NO: 96 - DNA, 80 bp, synthetic construct; identical to SEQ ID NO: 89
  misc_feature 1..80: note = EnhancerLAP2.92
  source 1..80: mol_type = other DNA

SEQ ID NO: 97 - DNA, 100 bp, synthetic construct
  misc_feature 1..100: note = EnhancerLAP2.93
  source 1..100: mol_type = other DNA
cgcgaaagcgctggaaacccaaaccccgccctcccgcgggggagggtcagtggcgtgacgtcacacgcgcggcgtaaggggcggggctcgcgcggcctag

SEQ ID NO: 98 - DNA, 90 bp, synthetic construct
  misc_feature 1..90: note = EnhancerLAP2.94
  source 1..90: mol_type = other DNA
aactaacatcaaagagagagagagaaatcgagattctgtcacctcaccttagatcccagcccaggcccccgcctccaaactggcccctca

SEQ ID NO: 99 - DNA, 80 bp, synthetic construct
  misc_feature 1..80: note = EnhancerLAP2.95
  source 1..80: mol_type = other DNA
actctgtggtgagggggaggggagtagtgacagacctctcgcgagacttcagcggcgctgctcacgtgtgccgggagggg

SEQ ID NO: 100 - DNA, 60 bp, synthetic construct
  misc_feature 1..60: note = EnhancerLAP2.96
  source 1..60: mol_type = other DNA
atgcgcaggcgcggctcagctacccccgcagcgtgggcggaaattaaaaaaaaaaaaaaa

SEQ ID NO: 101 - DNA, 70 bp, synthetic construct
  misc_feature 1..70: note = EnhancerLAP2.97
  source 1..70: mol_type = other DNA
gtccccggaagagaaagtgaaagtggcagtcccggaagtgctgcagaccgaacggaaaactgtcccctca

SEQ ID NO: 102 - DNA, 80 bp, synthetic construct
  misc_feature 1..80: note = EnhancerLAP2.98
  source 1..80: mol_type = other DNA
gctcgatcggtaccgccccgccccaggcccggcccggcccgattcggccgggtgacagcgcctgactcccgctcggctcc

SEQ ID NO: 103 - DNA, 100 bp, synthetic construct
  misc_feature 1..100: note = EnhancerLAP2.99
  source 1..100: mol_type = other DNA
cctgggatggggaaagtacccattacactgcagcttccacctcatcctgctcccttgccttcactaacggcagttcaacacctcctggtcaacttcacca

SEQ ID NO: 104 - DNA, 80 bp, synthetic construct
  misc_feature 1..80: note = EnhancerLAP2.100
  source 1..80: mol_type = other DNA
ggtaagtggcttaagtgtagtaggacggccggctaacggcttgtcactgggcagtgctgattcatgcttctgtccttccc

SEQ ID NO: 105 - DNA, 90 bp, synthetic construct
  misc_feature 1..90: note = EnhancerLAP2.101
  source 1..90: mol_type = other DNA
tcctcggccccgcccctgctacggtccccgcgccctcctccccctcttccgcttgggtctggtgaggccccgctgcggctgcggacgccg

SEQ ID NO: 106 - DNA, 80 bp, synthetic construct
  misc_feature 1..80: note = EnhancerLAP2.102
  source 1..80: mol_type = other DNA
tctgggtacatgaaagaatctggttgccgtttgatgcgcttgggaaacccagatcaccaccccacagggcgcccctaagt

SEQ ID NO: 107 - DNA, 100 bp, synthetic construct
  misc_feature 1..100: note = EnhancerLAP2.103
  source 1..100: mol_type = other DNA
ggcgagagctgcgaggaaagtgagggaaggatgcgcgcgctaagcgggacccacggcgatccgcgtaccgggctcagtcccacctgcccggccgcccggc

SEQ ID NO: 108 - DNA, 80 bp, synthetic construct
  misc_feature 1..80: note = EnhancerLAP.104
  source 1..80: mol_type = other DNA
gacgtgcgaagcccgagtcacgtggcctcccgggccggcgggggcgggcacatgcggccggccgccggcctcccgaccct

SEQ ID NO: 109 - DNA, 70 bp, synthetic construct
  misc_feature 1..70: note = EnhancerLAP.105
  source 1..70: mol_type = other DNA
aaaactgtgggtgtacataggtttgttatctaattttgtaataatggggaccaagctcagtctaccagta

SEQ ID NO: 110 - DNA, 100 bp, synthetic construct
  misc_feature 1..100: note = EnhancerLAP.106
  source 1..100: mol_type = other DNA
aaatggtaactatagctctcacctgcttcagggaaggaaagacaatttcattcttttttttttttttttttttttttttttttcgagacagggtttctct

SEQ ID NO: 111 - DNA, 80 bp, synthetic construct; identical to SEQ ID NO: 35
  misc_feature 1..80: note = EnhancerLAP.107
  source 1..80: mol_type = other DNA

SEQ ID NO: 112 - DNA, 70 bp, synthetic construct
  misc_feature 1..70: note = EnhancerLAP.108
  source 1..70: mol_type = other DNA
tgtggttagtatggctccgcgtgcggccgcggttccgtgagcggcacgtgggcctggtgggctggctcgg

SEQ ID NO: 113 - DNA, 80 bp, synthetic construct
  misc_feature 1..80: note = EnhancerLAP.109
  source 1..80: mol_type = other DNA
ccacatttccagcacgcctttctgtcaggatgcctgaccctccgccccgacactccagtaccagccaaacccccaaagct

SEQ ID NO: 114 - DNA, 100 bp, synthetic construct
  misc_feature 1..100: note = EnhancerLAP2.110
  source 1..100: mol_type = other DNA
gtgagtcatcttttataccacatggagacacacctgaaatggttaaggtttcttgaaatatttcagatgatactcatatttttcttttcttttaattcaa

SEQ ID NO: 115 - DNA, 90 bp, synthetic construct
  misc_feature 1..90: note = EnhancerLAP2.111
  source 1..90: mol_type = other DNA
actataaaataaatacatttgcattttaatcttaactttaaagcattttagacaaaaggacagcttaggagtcagataaatattcatact

SEQ ID NO: 116 - DNA, 100 bp, synthetic construct
  misc_feature 1..100: note = EnhancerLAP2.112
  source 1..100: mol_type = other DNA
caattcctacaataattcagaaatagaataaagatcatttaagaaaatgcaggtctcattcttggaaaaatttgaaaaacaaaacaaacaaacaaaaaaa

SEQ ID NO: 117 - DNA, 90 bp, synthetic construct
  misc_feature 1..90: note = EnhancerLAP2.113
  source 1..90: mol_type = other DNA
cctaggtgaacccgaggcaggagaggaaaggagaatgagcctgggcagtgagtcaacattagttgtgggaaataaatccaggcaaggagt

SEQ ID NO: 118 - DNA, 80 bp, synthetic construct; identical to SEQ ID NO: 53
  misc_feature 1..80: note = EnhancerLAP2.114
  source 1..80: mol_type = other DNA

SEQ ID NO: 119 - DNA, 60 bp, synthetic construct; identical to SEQ ID NO: 54
  misc_feature 1..60: note = EnhancerLAP2.115
  source 1..60: mol_type = other DNA

SEQ ID NO: 120 - DNA, 90 bp, synthetic construct
  misc_feature 1..90: note = EnhancerLAP2.116
  source 1..90: mol_type = other DNA
ctggttaggagatgggagacgtgacaaggggtgagtgggtggtgtggggtctagaggctgggcctggagagacagcttctctgcaccacc

SEQ ID NO: 121 - DNA, 100 bp, synthetic construct
  misc_feature 1..100: note = EnhancerLAP2.117
  source 1..100: mol_type = other DNA
atgcctgtagggggcaccacagcccggctctgggccacactgcccgtcctgcctctcctgctatgctgttccctccccactttaggcctgggccctttcc

SEQ ID NO: 122 - DNA, 90 bp, synthetic construct
  misc_feature 1..90: note = EnhancerLAP2.118
  source 1..90: mol_type = other DNA
accctaaggtgagaagacacgccctgagtcctcagagctgggaatagcatcccaaaccctcttccttgaccttgaaccgccacctgctct

SEQ ID NO: 123 - DNA, 100 bp, synthetic construct
  misc_feature 1..100: note = EnhancerLAP2.119
  source 1..100: mol_type = other DNA
caggtacctgcacgagggtcagaaggcatcacgtgggtgggtgggtggagggtgtgaggaacgaggacaccaatggcaggaaagatcagaagtatcctgt

SEQ ID NO: 124 - DNA, 70 bp, synthetic construct
  misc_feature 1..70: note = EnhancerLAP2.120
  source 1..70: mol_type = other DNA
ctatgagagtgggttaggggtcatgggtctgccttttatactaccactccctgacacgcccaccggccca

SEQ ID NO: 125 - DNA, 100 bp, synthetic construct
  misc_feature 1..100: note = EnhancerLAP2.121
  source 1..100: mol_type = other DNA
actccaatgtgagtctacccttctgccctgccctgggtgggggtggctagcattgacattgtatctcaccaatgcatcctcattcccctgggaccctaca

SEQ ID NO: 126 - DNA, 100 bp, synthetic construct
  misc_feature 1..100: note = EnhancerLAP2.122
  source 1..100: mol_type = other DNA
ctgcggggaaagggagaaaacaggccggggcggcgggtcagtaacgccgcgggcgctggggattcctgtccagcccggcccggccaatcgcaaatacaga

SEQ ID NO: 127 - DNA, 70 bp, synthetic construct
  misc_feature 1..70: note = EnhancerLAP2.123
  source 1..70: mol_type = other DNA
aaagacacgtaggtagagcgccgcccccgcctgcgcgccccctggcaccgggccgccccctcgggcccgg

SEQ ID NO: 128 - DNA, 90 bp, synthetic construct
  misc_feature 1..90: note = EnhancerLAP2.124
  source 1..90: mol_type = other DNA
tgagtgagtaggcggggcgtgtgaggacaggggcgggcctcggcgtgaagggggcgtgaccacaagtgggggtgtggtctgaagggatag

SEQ ID NO: 129 - DNA, 100 bp, synthetic construct
  misc_feature 1..100: note = EnhancerLAP2.125
  source 1..100: mol_type = other DNA
ggtgacgtgtggggcaaggctggggaaggagggatggaaatatggatggtggccccaggaactgtcacttctttctctagaactggggtcacctccccag

SEQ ID NO: 130 - DNA, 80 bp, synthetic construct; identical to SEQ ID NO: 89
  misc_feature 1..80: note = EnhancerLAP2.126
  source 1..80: mol_type = other DNA

SEQ ID NO: 131 - DNA, 70 bp, synthetic construct
  misc_feature 1..70: note = EnhancerLAP.127
  source 1..70: mol_type = other DNA
gatactcaggtactctgtccactctctcggaagctcctgcttcactggggtttttttgtttgtttgtttg

SEQ ID NO: 132 - DNA, 70 bp, synthetic construct
  misc_feature 1..70: note = EnhancerLAP2.128
  source 1..70: mol_type = other DNA
ggtgagaggctgcagagggagcggcttccttctcttcccccttccttcctctgactactagggggggtaa

SEQ ID NO: 133 - DNA, 90 bp, synthetic construct
  misc_feature 1..90: note = EnhancerLAP2.129
  source 1..90: mol_type = other DNA
tctctctctctctctctctctctctctctctctctctctctctgccacccccccctcgttcctctggctggctcggctgaagcgagaagg

SEQ ID NO: 134 - DNA, 90 bp, synthetic construct
  misc_feature 1..90: note = EnhancerLAP2.130
  source 1..90: mol_type = other DNA
aacaaaggagagccggatgtgtgggatatgaggctgaataaaaatggctgaaaagcaagcgaggctggagggcggccacacccagtgcta

SEQ ID NO: 135 - DNA, 90 bp, synthetic construct
  misc_feature 1..90: note = EnhancerLAP2.131
  source 1..90: mol_type = other DNA
gcggcgggtgagtccacgcttccgaacgcgtgaggaaaccgctcgacctgctacctcagctgcgggaggaggaggcggcggcggagaagc

SEQ ID NO: 136 - DNA, 70 bp, synthetic construct
  misc_feature 1..70: note = EnhancerLAP2.132
  source 1..70: mol_type = other DNA
ctcaaagaggtgagtggtcggctgcgacccctgcctcgggcgctaaggtcaccctgcccagtggtgggag

SEQ ID NO: 137 - DNA, 80 bp, synthetic construct; identical to SEQ ID NO: 99
  misc_feature 1..80: note = EnhancerLAP2.133
  source 1..80: mol_type = other DNA

SEQ ID NO: 138 - DNA, 100 bp, synthetic construct
  misc_feature 1..100: note = EnhancerLAP2.134
  source 1..100: mol_type = other DNA
ctgtggagggctgcggaggctctagaggggtggggctgggagcgcggaggtggcgaccagaggttcagtactagtttgtggtttagatagtctctttaca

SEQ ID NO: 139 - DNA, 80 bp, synthetic construct
  misc_feature 1..80: note = EnhancerLAP2.135
  source 1..80: mol_type = other DNA
cgggtgagtggggcgcggcggagcgcgaggcggcccgggcgggaaggcggcgccggcggggacgcaatggcggccacgcg

SEQ ID NO: 140 - DNA, 70 bp, synthetic construct
  misc_feature 1..70: note = EnhancerLAP2.136
  source 1..70: mol_type = other DNA
gatcccggtgaggaagcggcgggcagatcaggaagtggaaggggcgggacttgagggtgaatggcagcgg

SEQ ID NO: 141 - DNA, 60 bp, synthetic construct; identical to SEQ ID NO: 100
  misc_feature 1..60: note = EnhancerLAP2.137
  source 1..60: mol_type = other DNA

SEQ ID NO: 142 - DNA, 101 bp, synthetic construct
  misc_feature 1..101: note = EnhancerLAP2.138
  source 1..101: mol_type = other DNA
tgcgccaggtgcgccagcggggcgcgcgacggggcgcggggagggcggactcctgatggctttgcctgctcggtgtccggccgcggcctggcctcacccga

SEQ ID NO: 143 - DNA, 100 bp, synthetic construct
  misc_feature 1..100: note = EnhancerLAP2.139
  source 1..100: mol_type = other DNA
ctcctcctgatccctccccttggttatttaggagattagagttcattaattaaatcctgtgcttagatcgaactgtaacactattccaatcacatttatc

SEQ ID NO: 144 - DNA, 60 bp, synthetic construct
  misc_feature 1..60: note = EnhancerLAP2.140
  source 1..60: mol_type = other DNA
cccccctccgccccccgcacctgcagcggctcgcgcccagggccccgccccgccagccgc

SEQ ID NO: 145 - DNA, 100 bp, synthetic construct
  misc_feature 1..100: note = EnhancerLAP2.141
  source 1..100: mol_type = other DNA
ggggcaggcccggggcccggggcggggcggccgtcaggcgcgggctgcggtgctaggccagacggcggggcacgcggtgcggaccggccggggacgcgca

SEQ ID NO: 146 - DNA, 70 bp, synthetic construct
  misc_feature 1..70: note = EnhancerLAP22.142
  source 1..70: mol_type = other DNA
gcagttttagggatgttcacagagaagagagacacaagcaaggggcggacacacaaggggcgtggttctt

SEQ ID NO: 147 - DNA, 80 bp, synthetic construct
  misc_feature 1..80: note = EnhancerLAP2.143
  source 1..80: mol_type = other DNA
tgtccaggtaggaatccacccggctgggcaggctctcggcccacgcggcttcctctccccacgcacctcctccgcgccgt

SEQ ID NO: 148 - DNA, 90 bp, synthetic construct
  misc_feature 1..90: note = EnhancerLAP2.144
  source 1..90: mol_type = other DNA
ctgacagcacctgaagtggaaggaacaaggatgctcaggggacaacatcggggtaacgaccccacctgccctgtcccgcagctcgcctca

SEQ ID NO: 149 - DNA, 70 bp, synthetic construct
  misc_feature 1..70: note = EnhancerLAP2.145
  source 1..70: mol_type = other DNA
gcgtctttggtaagctggtcctccctacccaccttccctgccttcccctcctccctgtctggcagcctcc

SEQ ID NO: 150 - DNA, 80 bp, synthetic construct
  misc_feature 1..80: note = EnhancerLAP2.146
  source 1..80: mol_type = other DNA
gtgaggaccacgggggacagggaagagagatgtgtggcgtccgcactgcgggaggctggcatctgccgcaccccccacct

SEQ ID NO: 151 - DNA, 80 bp, synthetic construct; identical to SEQ ID NO: 102
  misc_feature 1..80: note = EnhancerLAP2.147
  source 1..80: mol_type = other DNA

SEQ ID NO: 152 - DNA, 70 bp, synthetic construct
  misc_feature 1..70: note = EnhancerLAP2.148
  source 1..70: mol_type = other DNA
tggtactaagtatcaaatggaaagggaaagcgagcaggcatatttctgccatggcagaaacagctctcat

SEQ ID NO: 153 - DNA, 80 bp, synthetic construct; identical to SEQ ID NO: 104
  misc_feature 1..80: note = EnhancerLAP2.149
  source 1..80: mol_type = other DNA

SEQ ID NO: 154 - DNA, 90 bp, synthetic construct; identical to SEQ ID NO: 105
  misc_feature 1..90: note = EnhancerLAP2.150
  source 1..90: mol_type = other DNA

SEQ ID NO: 155 - DNA, 60 bp, synthetic construct
  misc_feature 1..60: note = EnhancerLAP2.151
  source 1..60: mol_type = other DNA
ctcgcgggcgctgtccatggtgcctgcgcgccagccgcacccggccggcgctcgctagct

SEQ ID NO: 156 - DNA, 60 bp, synthetic construct
  misc_feature 1..60: note = EnhancerLAP2.152
  source 1..60: mol_type = other DNA
caagaagagtgagttgggacaccttgggtggcggctaaggctaggggcggggaactccta

SEQ ID NO: 157 - DNA, 90 bp, synthetic construct
  misc_feature 1..90: note = EnhancerLAP2.153
  source 1..90: mol_type = other DNA
gtgagtaaatcctgcgggtaataaacgcggaagtatgaagttgtgccgcaccagcctcagctacccaaattgttggctgcttttgtgatt

SEQ ID NO: 158 - DNA, 90 bp, synthetic construct
  misc_feature 1..90: note = EnhancerLAP2.154
  source 1..90: mol_type = other DNA
ctttcactgtggaatatacacggggttcctcatagctaccatcaccagacccagttccccacaccttggcctgcgcaggcgcccctcact

SEQ ID NO: 159 - DNA, 90 bp, synthetic construct
  misc_feature 1..90: note = EnhancerLAP2.155
  source 1..90: mol_type = other DNA
attgcatttcctctttccccgcagaacaagatggcggcggtcacgtgggaagggacagccggtacaggttggcgcgtggtccccgccgcc

SEQ ID NO: 160 - DNA, 90 bp, synthetic construct
  misc_feature 1..90: note = EnhancerLAP2.156
  source 1..90: mol_type = other DNA
caaggccatggataggaaaaatccagcagcagatggaaaggcctggatcctagacccaacccgaacccagaggaccccaggggtcccgct

SEQ ID NO: 161 - DNA, 51 bp, synthetic construct
  misc_feature 1..51: note = EnhancerLAP2.157
  source 1..51: mol_type = other DNA
gcagcagctggcggataattaattagtttaaaagtttgggacagatggcag

SEQ ID NO: 162 - DNA, 23 bp, synthetic construct
  misc_feature 1..23: note = EnhancerLAP2.158
  source 1..23: mol_type = other DNA
aacatatgtcgcagcagctggcg

SEQ ID NO: 163 - DNA, 69 bp, synthetic construct
  misc_feature 1..69: note = EnhancerLAP2.159
  source 1..69: mol_type = other DNA
gcagcagctggcgctttgttatgcaaatgtaaacaaggggtaattagctctggagcgttaattaatgta

SEQ ID NO: 164 - DNA, 21 bp, synthetic construct
  misc_feature 1..21: note = EnhancerLAP2.160
  source 1..21: mol_type = other DNA
gcagcagctggcggcaattaa

SEQ ID NO: 165 - DNA, 67 bp, synthetic construct
  misc_feature 1..67: note = EnhancerLAP2.161
  source 1..67: mol_type = other DNA
gcagcagctggcggataattaattagtttaaaagtttgcgaattaattaaaaatctgtttacttagg

SEQ ID NO: 166 - DNA, 40 bp, synthetic construct
  misc_feature 1..40: note = EnhancerLAP2.162
  source 1..40: mol_type = other DNA
gcagcagctggcggggaaatccccgaattaattaaaaatc

SEQ ID NO: 167 - DNA, 29 bp, synthetic construct
  misc_feature 1..29: note = EnhancerLAP2.163
  source 1..29: mol_type = other DNA
gcagcagctggcgagggggattagctgcc

SEQ ID NO: 168 - DNA, 101 bp, synthetic construct
  misc_feature 1..101: note = EnhancerLAP2.164
  source 1..101: mol_type = other DNA
gcagcagctggcggataattaattagtttaaaagtttgcgaattaattaaaaatcagtttttaattaatttgtgtttacttaggtgtagggattaattgtc

SEQ ID NO: 169 - DNA, 40 bp, synthetic construct
  misc_feature 1..40: note = EnhancerLAP2.165
  source 1..40: mol_type = other DNA
gcagcagctggcggggaaatcccagtttttaattaatttg

SEQ ID NO: 170 - DNA, 25 bp, synthetic construct
  misc_feature 1..25: note = EnhancerLAP2.166
  source 1..25: mol_type = other DNA
aacatatgtcaacaatttcagtgtt

SEQ ID NO: 171 - DNA, 85 bp, synthetic construct
  misc_feature 1..85: note = EnhancerLAP2.167
  source 1..85: mol_type = other DNA
gcagcagctggcggataattaattagtttaaaagtttgaacatatgtcaaattaattaatcatccattagcagaacagatggcag

SEQ ID NO: 172 - DNA, 49 bp, synthetic construct
  misc_feature 1..49: note = EnhancerLAP2.168
  source 1..49: mol_type = other DNA
aacatatgtcaacaatttcagtgttatccattagcaaaattaattaatc

SEQ ID NO: 173 - DNA, 28 bp, synthetic construct
  misc_feature 1..28: note = EnhancerLAP2.169
  source 1..28: mol_type = other DNA
tcagccaatcagcgaggccgcggacgct

SEQ ID NO: 174 - DNA, 26 bp, synthetic construct
  misc_feature 1..26: note = EnhancerLAP2.170
  source 1..26: mol_type = other DNA
ccgcgaggaggcaggccgcggacgct

SEQ ID NO: 175 - DNA, 46 bp, synthetic construct
  misc_feature 1..46: note = EnhancerLAP2.171
  source 1..46: mol_type = other DNA
tggtgccctctggtggtcttctagaggaattgcagccgcggacgct

SEQ ID NO: 176 - DNA, 16 bp, synthetic construct
  misc_feature 1..16: note = EnhancerLAP2.172
  source 1..16: mol_type = other DNA
tcagccaatcagcgag

SEQ ID NO: 177 - DNA, 484 bp, synthetic construct
  misc_feature 1..484: note = EnhancerLAP2.1
  source 1..484: mol_type = other DNA
cccaggaactcaggcttaccaggaaccccaatatttaacataagggtagcaactcagtaggaatactaagaatccaaatgatccccggtccgcgctccgcccacccatcacagcagccgcggacgctgcgcgccggagcggtccatctcgccagccagccaaccagccgagccgcccagccgacccgagagccccgagagccagactccctcagccatagaagacaccgggcgggagagacggactgaaaaaatatatctttttttattttgtctgggcctggagacccgcagcaggagcggaggtgggtgcggggccgggagccggagcaggaccgggaacaggaacaggaacaggaacaggaacaggaacaggagtggggccgggagcaggagcaggagcgggagccgaagtgggggcaggagcggcggcggccgcagcagcaacagggtcgccccagtccgcggcgaggaagagggagctc

SEQ ID NO: 178 - DNA, 504 bp, synthetic construct
  misc_feature 1..504: note = EnhancerLAP2.2
  source 1..504: mol_type = other DNA
tgtctcctgaaaagggaagcactgagtgactgactgcagggctttcagcccctcccttagccccaggagtgggtgctcctttctagacatcagatcccctatccccggtccgcgctccgcccacccatcacagcagccgcggacgctgcgcgccggagcggtccatctcgccagccagccaaccagccgagccgcccagccgacccgagagccccgagagccagactccctcagccatagaagacaccgggcgggagagacggactgaaaaaatatatctttttttattttgtctgggcctggagacccgcagcaggagcggaggtgggtgcggggccgggagccggagcaggaccgggaacaggaacaggaacaggaacaggaacaggaacaggagtggggccgggagcaggagcaggagcgggagccgaagtgggggcaggagcggcggcggccgcagcagcaacagggtcgccccagtccgcggcgaggaagagggagctc

SEQ ID NO: 179 - DNA, 100 bp, synthetic construct; identical to SEQ ID NO: 7
  misc_feature 1..100: note = EnhancerLAP2.3
  source 1..100: mol_type = other DNA

SEQ ID NO: 180 - DNA, 484 bp, synthetic construct
  misc_feature 1..484: note = EnhancerLAP2.4
  source 1..484: mol_type = other DNA
agttaaaagaaaaaagaaaagacagcaagaaatcaaccttggtatcaagtgagaaactctaaggctgatgagcctcttacatccccggtccgcgctccgcccacccatcacagcagccgcggacgctgcgcgccggagcggtccatctcgccagccagccaaccagccgagccgcccagccgacccgagagccccgagagccagactccctcagccatagaagacaccgggcgggagagacggactgaaaaaatatatctttttttattttgtctgggcctggagacccgcagcaggagcggaggtgggtgcggggccgggagccggagcaggaccgggaacaggaacaggaacaggaacaggaacaggaacaggagtggggccgggagcaggagcaggagcgggagccgaagtgggggcaggagcggcggcggccgcagcagcaacagggtcgccccagtccgcggcgaggaagagggagctc

SEQ ID NO: 181 - DNA, 484 bp, synthetic construct
  misc_feature 1..484: note = EnhancerLAP2.5
  source 1..484: mol_type = other DNA
gtaaggaagatgtcctttatggtatatttggccgtgttatttgtggctgcagagatggaggtgggcgttcacgtcgtgatatccccggtccgcgctccgcccacccatcacagcagccgcggacgctgcgcgccggagcggtccatctcgccagccagccaaccagccgagccgcccagccgacccgagagccccgagagccagactccctcagccatagaagacaccgggcgggagagacggactgaaaaaatatatctttttttattttgtctgggcctggagacccgcagcaggagcggaggtgggtgcggggccgggagccggagcaggaccgggaacaggaacaggaacaggaacaggaacaggaacaggagtggggccgggagcaggagcaggagcgggagccgaagtgggggcaggagcggcggcggccgcagcagcaacagggtcgccccagtccgcggcgaggaagagggagctc

SEQ ID NO: 182 - DNA, 504 bp, synthetic construct
  misc_feature 1..504: note = EnhancerLAP2.6
  source 1..504: mol_type = other DNA
ctgaattccttcacctttgctacctttttctgctcttgctggctgatgcaactcagctgttctggctcagactcctctcctagctgactgattcaatctgatccccggtccgcgctccgcccacccatcacagcagccgcggacgctgcgcgccggagcggtccatctcgccagccagccaaccagccgagccgcccagccgacccgagagccccgagagccagactccctcagccatagaagacaccgggcgggagagacggactgaaaaaatatatctttttttattttgtctgggcctggagacccgcagcaggagcggaggtgggtgcggggccgggagccggagcaggaccgggaacaggaacaggaacaggaacaggaacaggaacaggagtggggccgggagcaggagcaggagcgggagccgaagtgggggcaggagcggcggcggccgcagcagcaacagggtcgccccagtccgcggcgaggaagagggagctc

SEQ ID NO: 183 - DNA, 464 bp, synthetic construct
  misc_feature 1..464: note = EnhancerLAP2.7
  source 1..464: mol_type = other DNA
atgctgtaaagtagaagagataggatgagtttgattgttacagccaaccaacagaaatatatccccggtccgcgctccgcccacccatcacagcagccgcggacgctgcgcgccggagcggtccatctcgccagccagccaaccagccgagccgcccagccgacccgagagccccgagagccagactccctcagccatagaagacaccgggcgggagagacggactgaaaaaatatatctttttttattttgtctgggcctggagacccgcagcaggagcggaggtgggtgcggggccgggagccggagcaggaccgggaacaggaacaggaacaggaacaggaacaggaacaggagtggggccgggagcaggagcaggagcgggagccgaagtgggggcaggagcggcggcggccgcagcagcaacagggtcgccccagtccgcggcgaggaagagggagctc

SEQ ID NO: 184 - DNA, 494 bp, synthetic construct
  misc_feature 1..494: note = EnhancerLAP2.8
  source 1..494: mol_type = other DNA
gggctggaaaataaaaagtggggttgttgtggttaggggacaattagggaagtgatgagggaggtgggaggagcgagggtggagagctcaatccccggtccgcgctccgcccacccatcacagcagccgcggacgctgcgcgccggagcggtccatctcgccagccagccaaccagccgagccgcccagccgacccgagagccccgagagccagactccctcagccatagaagacaccgggcgggagagacggactgaaaaaatatatctttttttattttgtctgggcctggagacccgcagcaggagcggaggtgggtgcggggccgggagccggagcaggaccgggaacaggaacaggaacaggaacaggaacaggaacaggagtggggccgggagcaggagcaggagcgggagccgaagtgggggcaggagcggcggcggccgcagcagcaacagggtcgccccagtccgcggcgaggaagagggagctc

SEQ ID NO: 185 - DNA, 484 bp, synthetic construct
  misc_feature 1..484: note = EnhancerLAP2.9
  source 1..484: mol_type = other DNA
tgggcaggtatgcatcacctttttagtggatgaggagagggagagccatgatggcacggtgccatgacagtgtcctctgtatccccggtccgcgctccgcccacccatcacagcagccgcggacgctgcgcgccggagcggtccatctcgccagccagccaaccagccgagccgcccagccgacccgagagccccgagagccagactccctcagccatagaagacaccgggcgggagagacggactgaaaaaatatatctttttttattttgtctgggcctggagacccgcagcaggagcggaggtgggtgcggggccgggagccggagcaggaccgggaacaggaacaggaacaggaacaggaacaggaacaggagtggggccgggagcaggagcaggagcgggagccgaagtgggggcaggagcggcggcggccgcagcagcaacagggtcgccccagtccgcggcgaggaagagggagctc

SEQ ID NO: 186 - DNA, 504 bp, synthetic construct
  misc_feature 1..504: note = EnhancerLAP2.10
  source 1..504: mol_type = other DNA
accgttgctagaaggggaaggaaggaggcagaggacaggtgagctcaccccaggccagctcagaaagtaccacaccaactctgcccacctaactctccttatccccggtccgcgctccgcccacccatcacagcagccgcggacgctgcgcgccggagcggtccatctcgccagccagccaaccagccgagccgcccagccgacccgagagccccgagagccagactccctcagccatagaagacaccgggcgggagagacggactgaaaaaatatatctttttttattttgtctgggcctggagacccgcagcaggagcggaggtgggtgcggggccgggagccggagcaggaccgggaacaggaacaggaacaggaacaggaacaggaacaggagtggggccgggagcaggagcaggagcgggagccgaagtgggggcaggagcggcggcggccgcagcagcaacagggtcgccccagtccgcggcgaggaagagggagctc

SEQ ID NO: 187 - DNA, 464 bp, synthetic construct
  misc_feature 1..464: note = EnhancerLAP2.11
  source 1..464: mol_type = other DNA
aggtccctgagcagaaagggacagagagcaggtggagccagctctgtctccctctgttttatccccggtccgcgctccgcccacccatcacagcagccgcggacgctgcgcgccggagcggtccatctcgccagccagccaaccagccgagccgcccagccgacccgagagccccgagagccagactccctcagccatagaagacaccgggcgggagagacggactgaaaaaatatatctttttttattttgtctgggcctggagacccgcagcaggagcggaggtgggtgcggggccgggagccggagcaggaccgggaacaggaacaggaacaggaacaggaacaggaacaggagtggggccgggagcaggagcaggagcgggagccgaagtgggggcaggagcggcggcggccgcagcagcaacagggtcgccccagtccgcggcgaggaagagggagctc

SEQ ID NO: 188 - DNA, 484 bp, synthetic construct
  misc_feature 1..484: note = EnhancerLAP2.12
  source 1..484: mol_type = other DNA
ccctggggaacaaaatgctgtgtgaagctacacatctggtcattctctgagtccctctgtatgtaaccctggttgccaccatccccggtccgcgctccgcccacccatcacagcagccgcggacgctgcgcgccggagcggtccatctcgccagccagccaaccagccgagccgcccagccgacccgagagccccgagagccagactccctcagccatagaagacaccgggcgggagagacggactgaaaaaatatatctttttttattttgtctgggcctggagacccgcagcaggagcggaggtgggtgcggggccgggagccggagcaggaccgggaacaggaacaggaacaggaacaggaacaggaacaggagtggggccgggagcaggagcaggagcgggagccgaagtgggggcaggagcggcggcggccgcagcagcaacagggtcgccccagtccgcggcgaggaagagggagctc

SEQ ID NO: 189 - DNA, 494 bp, synthetic construct
  misc_feature 1..494: note = EnhancerLAP2.13
  source 1..494: mol_type = other DNA
gtctcacctaaagggacaaaaaagaaatatgagcaaggagctagggttggagagagctagggttggacctggtaagaagctgaccctagaatccccggtccgcgctccgcccacccatcacagcagccgcggacgctgcgcgccggagcggtccatctcgccagccagccaaccagccgagccgcccagccgacccgagagccccgagagccagactccctcagccatagaagacaccgggcgggagagacggactgaaaaaatatatctttttttattttgtctgggcctggagacccgcagcaggagcggaggtgggtgcggggccgggagccggagcaggaccgggaacaggaacaggaacaggaacaggaacaggaacaggagtggggccgggagcaggagcaggagcgggagccgaagtgggggcaggagcggcggcggccgcagcagcaacagggtcgccccagtccgcggcgaggaagagggagctc

SEQ ID NO: 190 - DNA, 484 bp, synthetic construct
  misc_feature 1..484: note = EnhancerLAP2.14
  source 1..484: mol_type = other DNA
ctggaggaggagaaatacaggctatgttgggtcatgccgtggggccatgagtaccccttggaatggtgggaggggtacccatccccggtccgcgctccgcccacccatcacagcagccgcggacgctgcgcgccggagcggtccatctcgccagccagccaaccagccgagccgcccagccgacccgagagccccgagagccagactccctcagccatagaagacaccgggcgggagagacggactgaaaaaatatatctttttttattttgtctgggcctggagacccgcagcaggagcggaggtgggtgcggggccgggagccggagcaggaccgggaacaggaacaggaacaggaacaggaacaggaacaggagtggggccgggagcaggagcaggagcgggagccgaagtgggggcaggagcggcggcggccgcagcagcaacagggtcgccccagtccgcggcgaggaagagggagctc

SEQ ID NO: 191 - DNA, 494 bp, synthetic construct
  misc_feature 1..494: note = EnhancerLAP2.15
  source 1..494: mol_type = other DNA
gtggaacccacagatatagagggtcaatgacaggtacactgtttaaaggtataacattacaaaacaaaacaaaactagcacactcataaaatccccggtccgcgctccgcccacccatcacagcagccgcggacgctgcgcgccggagcggtccatctcgccagccagccaaccagccgagccgcccagccgacccgagagccccgagagccagactccctcagccatagaagacaccgggcgggagagacggactgaaaaaatatatctttttttattttgtctgggcctggagacccgcagcaggagcggaggtgggtgcggggccgggagccggagcaggaccgggaacaggaacaggaacaggaacaggaacaggaacaggagtggggccgggagcaggagcaggagcgggagccgaagtgggggcaggagcggcggcggccgcagcagcaacagggtcgccccagtccgcggcgaggaagagggagctc

SEQ ID NO: 192 - DNA, 474 bp, synthetic construct
  misc_feature 1..474: note = EnhancerLAP2.16
  source 1..474: mol_type = other DNA
atacttgggacttccagaatgctatccctaccctgcctgccaggcatcagtgagtatgatgacatttggaatccccggtccgcgctccgcccacccatcacagcagccgcggacgctgcgcgccggagcggtccatctcgccagccagccaaccagccgagccgcccagccgacccgagagccccgagagccagactccctcagccatagaagacaccgggcgggagagacggactgaaaaaatatatctttttttattttgtctgggcctggagacccgcagcaggagcggaggtgggtgcggggccgggagccggagcaggaccgggaacaggaacaggaacaggaacaggaacaggaacaggagtggggccgggagcaggagcaggagcgggagccgaagtgggggcaggagcggcggcggccgcagcagcaacagggtcgccccagtccgcggcgaggaagagggagctc

SEQ ID NO: 193 - DNA, 474 bp, synthetic construct
  misc_feature 1..474: note = EnhancerLAP2.17
  source 1..474: mol_type = other DNA
cgccttccgccccgcccgcgccccgcctcgtccgcccgcgtcccggtctccgccgcacgcccgcagagtgatccccggtccgcgctccgcccacccatcacagcagccgcggacgctgcgcgccggagcggtccatctcgccagccagccaaccagccgagccgcccagccgacccgagagccccgagagccagactccctcagccatagaagacaccgggcgggagagacggactgaaaaaatatatctttttttattttgtctgggcctggagacccgcagcaggagcggaggtgggtgcggggccgggagccggagcaggaccgggaacaggaacaggaacaggaacaggaacaggaacaggagtggggccgggagcaggagcaggagcgggagccgaagtgggggcaggagcggcggcggccgcagcagcaacagggtcgccccagtccgcggcgaggaagagggagctc

SEQ ID NO: 194 - DNA, 494 bp, synthetic construct
  misc_feature 1..494: note = EnhancerLAP2.18
  source 1..494: mol_type = other DNA
ccaggaggtgagggcagccaccccacagcccctgcatccctgtccccaccccatggcttttcccacactgacatcatgtccccgtctcctatccccggtccgcgctccgcccacccatcacagcagccgcggacgctgcgcgccggagcggtccatctcgccagccagccaaccagccgagccgcccagccgacccgagagccccgagagccagactccctcagccatagaagacaccgggcgggagagacggactgaaaaaatatatctttttttattttgtctgggcctggagacccgcagcaggagcggaggtgggtgcggggccgggagccggagcaggaccgggaacaggaacaggaacaggaacaggaacaggaacaggagtggggccgggagcaggagcaggagcgggagccgaagtgggggcaggagcggcggcggccgcagcagcaacagggtcgccccagtccgcggcgaggaagagggagctc

SEQ ID NO: 195 - DNA, 474 bp, synthetic construct
  misc_feature 1..474: note = EnhancerLAP2.19
  source 1..474: mol_type = other DNA
gggtaactccgcggggctcggggcgggctggccgcgcgggtcggcgtcgctgcacctcagcgagcgcctgatccccggtccgcgctccgcccacccatcacagcagccgcggacgctgcgcgccggagcggtccatctcgccagccagccaaccagccgagccgcccagccgacccgagagccccgagagccagactccctcagccatagaagacaccgggcgggagagacggactgaaaaaatatatctttttttattttgtctgggcctggagacccgcagcaggagcggaggtgggtgcggggccgggagccggagcaggaccgggaacaggaacaggaacaggaacaggaacaggaacaggagtggggccgggagcaggagcaggagcgggagccgaagtgggggcaggagcggcggcggccgcagcagcaacagggtcgccccagtccgcggcgaggaagagggagctc

SEQ ID NO: 196 - DNA, 504 bp, synthetic construct
  misc_feature 1..504: note = EnhancerLAP2.20
  source 1..504: mol_type = other DNA
tgcagcagtcccaagttcaagaaggttccaagagataaggagggcccagtgctatctctaggagccagtgccaaggtttgcagccaaaatcaccgagaccatccccggtccgcgctccgcccacccatcacagcagccgcggacgctgcgcgccggagcggtccatctcgccagccagccaaccagccgagccgcccagccgacccgagagccccgagagccagactccctcagccatagaagacaccgggcgggagagacggactgaaaaaatatatctttttttattttgtctgggcctggagacccgcagcaggagcggaggtgggtgcggggccgggagccggagcaggaccgggaacaggaacaggaacaggaacaggaacaggaacaggagtggggccgggagcaggagcaggagcgggagccgaagtgggggcaggagcggcggcggccgcagcagcaacagggtcgccccagtccgcggcgaggaagagggagctc

SEQ ID NO: 197 - DNA, 484 bp, synthetic construct
  misc_feature 1..484: note = EnhancerLAP2.21
  source 1..484: mol_type = other DNA
ggcgcaagggtcccgggactggtcaccagaggggaacttagggtgaggagggcagcgctcctcggtgtggctctaactcaatccccggtccgcgctccgcccacccatcacagcagccgcggacgctgcgcgccggagcggtccatctcgccagccagccaaccagccgagccgcccagccgacccgagagccccgagagccagactccctcagccatagaagacaccgggcgggagagacggactgaaaaaatatatctttttttattttgtctgggcctggagacccgcagcaggagcggaggtgggtgcggggccgggagccggagcaggaccgggaacaggaacaggaacaggaacaggaacaggaacaggagtggggccgggagcaggagcaggagcgggagccgaagtgggggcaggagcggcggcggccgcagcagcaacagggtcgccccagtccgcggcgaggaagagggagctc

SEQ ID NO: 198 - DNA, 464 bp, synthetic construct
  misc_feature 1..464: note = EnhancerLAP2.22
  source 1..464: mol_type = other DNA
tatgaggtagcatctttggtttccttcatcttattactgagcgctggggccagagaccttatccccggtccgcgctccgcccacccatcacagcagccgcggacgctgcgcgccggagcggtccatctcgccagccagccaaccagccgagccgcccagccgacccgagagccccgagagccagactccctcagccatagaagacaccgggcgggagagacggactgaaaaaatatatctttttttattttgtctgggcctggagacccgcagcaggagcggaggtgggtgcggggccgggagccggagcaggaccgggaacaggaacaggaacaggaacaggaacaggaacaggagtggggccgggagcaggagcaggagcgggagccgaagtgggggcaggagcggcggcggccgcagcagcaacagggtcgccccagtccgcggcgaggaagagggagctc

SEQ ID NO: 199 - DNA, 494 bp, synthetic construct
  misc_feature 1..494: note = EnhancerLAP2.23
  source 1..494: mol_type = other DNA
actaaatagggatggatatgcagggcggaggtaaggaccctgcagccaaactagcctcgttggactaagggggctacaatgtggtgtctaatccccggtccgcgctccgcccacccatcacagcagccgcggacgctgcgcgccggagcggtccatctcgccagccagccaaccagccgagccgcccagccgacccgagagccccgagagccagactccctcagccatagaagacaccgggcgggagagacggactgaaaaaatatatctttttttattttgtctgggcctggagacccgcagcaggagcggaggtgggtgcggggccgggagccggagcaggaccgggaacaggaacaggaacaggaacaggaacaggaacaggagtggggccgggagcaggagcaggagcgggagccgaagtgggggcaggagcggcggcggccgcagcagcaacagggtcgccccagtccgcggcgaggaagagggagctc

SEQ ID NO: 200 - DNA, 474 bp, synthetic construct
  misc_feature 1..474: note = EnhancerLAP2.24
  source 1..474: mol_type = other DNA
ggtctgtggtgaccctgtccctctgtacatacctctgtcatatttcccccacccctccatgctatctgctatccccggtccgcgctccgcccacccatcacagcagccgcggacgctgcgcgccggagcggtccatctcgccagccagccaaccagccgagccgcccagccgacccgagagccccgagagccagactccctcagccatagaagacaccgggcgggagagacggactgaaaaaatatatctttttttattttgtctgggcctggagacccgcagcaggagcggaggtgggtgcggggccgggagccggagcaggaccgggaacaggaacaggaacaggaacaggaacaggaacaggagtggggccgggagcaggagcaggagcgggagccgaagtgggggcaggagcggcggcggccgcagcagcaacagggtcgccccagtccgcggcgaggaagagggagctc

SEQ ID NO: 201 - DNA, 474 bp, synthetic construct
  misc_feature 1..474: note = EnhancerLAP2.25
  source 1..474: mol_type = other DNA
ccgggacaggtccgtgcggatcgggagaggccagcgcggcccgggctgaggtggatggtggggtcaggggatccccggtccgcgctccgcccacccatcacagcagccgcggacgctgcgcgccggagcggtccatctcgccagccagccaaccagccgagccgcccagccgacccgagagccccgagagccagactccctcagccatagaagacaccgggcgggagagacggactgaaaaaatatatctttttttattttgtctgggcctggagacccgcagcaggagcggaggtgggtgcggggccgggagccggagcaggaccgggaacaggaacaggaacaggaacaggaacaggaacaggagtggggccgggagcaggagcaggagcgggagccgaagtgggggcaggagcggcggcggccgcagcagcaacagggtcgccccagtccgcggcgaggaagagggagctc

SEQ ID NO: 202 - DNA, 494 bp, synthetic construct
  misc_feature 1..494: note = EnhancerLAP2.26
  source 1..494: mol_type = other DNA
tctgtggcaaggggaggcgggcgcggaggaggtgagcaggcatggcagtgagcccctcctagccctgcctcccctcttccatccccaccaatccccggtccgcgctccgcccacccatcacagcagccgcggacgctgcgcgccggagcggtccatctcgccagccagccaaccagccgagccgcccagccgacccgagagccccgagagccagactccctcagccatagaagacaccgggcgggagagacggactgaaaaaatatatctttttttattttgtctgggcctggagacccgcagcaggagcggaggtgggtgcggggccgggagccggagcaggaccgggaacaggaacaggaacaggaacaggaacaggaacaggagtggggccgggagcaggagcaggagcgggagccgaagtgggggcaggagcggcggcggccgcagcagcaacagggtcgccccagtccgcggcgaggaagagggagctc

SEQ ID NO: 203 - DNA, 494 bp, synthetic construct
  misc_feature 1..494: note = EnhancerLAP2.27
  source 1..494: mol_type = other DNA
aggtctgtgtggggtgagtaggggtgaggtaagtggacactggggttgccttcccagggtccctggcagagtagccctctgttctcttacatccccggtccgcgctccgcccacccatcacagcagccgcggacgctgcgcgccggagcggtccatctcgccagccagccaaccagccgagccgcccagccgacccgagagccccgagagccagactccctcagccatagaagacaccgggcgggagagacggactgaaaaaatatatctttttttattttgtctgggcctggagacccgcagcaggagcggaggtgggtgcggggccgggagccggagcaggaccgggaacaggaacaggaacaggaacaggaacaggaacaggagtggggccgggagcaggagcaggagcgggagccgaagtgggggcaggagcggcggcggccgcagcagcaacagggtcgccccagtccgcggcgaggaagagggagctc

SEQ ID NO: 204 - DNA, 504 bp, synthetic construct
  misc_feature 1..504: note = EnhancerLAP2.28
  source 1..504: mol_type = other DNA
gtgaggtaggttttccgccttacctgtcctgtgggggtggatggagcaggtctggggccaaggccgattgcagggcccatgcagcactcctctgggagctatccccggtccgcgctccgcccacccatcacagcagccgcggacgctgcgcgccggagcggtccatctcgccagccagccaaccagccgagccgcccagccgacccgagagccccgagagccagactccctcagccatagaagacaccgggcgggagagacggactgaaaaaatatatctttttttattttgtctgggcctggagacccgcagcaggagcggaggtgggtgcggggccgggagccggagcaggaccgggaacaggaacaggaacaggaacaggaacaggaacaggagtggggccgggagcaggagcaggagcgggagccgaagtgggggcaggagcggcggcggccgcagcagcaacagggtcgccccagtccgcggcgaggaagagggagctc

SEQ ID NO: 205 - DNA, 464 bp, synthetic construct
  misc_feature 1..464: note = EnhancerLAP2.29
  source 1..464: mol_type = other DNA
gtgggtggggtcagggactttcttcaggaggttaaccttgttacacgtgaaattttcttcatccccggtccgcgctccgcccacccatcacagcagccgcggacgctgcgcgccggagcggtccatctcgccagccagccaaccagccgagccgcccagccgacccgagagccccgagagccagactccctcagccatagaagacaccgggcgggagagacggactgaaaaaatatatctttttttattttgtctgggcctggagacccgcagcaggagcggaggtgggtgcggggccgggagccggagcaggaccgggaacaggaacaggaacaggaacaggaacaggaacaggagtggggccgggagcaggagcaggagcgggagccgaagtgggggcaggagcggcggcggccgcagcagcaacagggtcgccccagtccgcggcgaggaagagggagctc

SEQ ID NO: 206 - DNA, 494 bp, synthetic construct
  misc_feature 1..494: note = EnhancerLAP2.30
  source 1..494: mol_type = other DNA
acctgttgtggggagaacacagaaaggaggggtaagtcggatctcttggagatgcccaccccctgagcccacctaccccactggtgccatatccccggtccgcgctccgcccacccatcacagcagccgcggacgctgcgcgccggagcggtccatctcgccagccagccaaccagccgagccgcccagccgacccgagagccccgagagccagactccctcagccatagaagacaccgggcgggagagacggactgaaaaaatatatctttttttattttgtctgggcctggagacccgcagcaggagcggaggtgggtgcggggccgggagccggagcaggaccgggaacaggaacaggaacaggaacaggaacaggaacaggagtggggccgggagcaggagcaggagcgggagccgaagtgggggcaggagcggcggcggccgcagcagcaacagggtcgccccagtccgcggcgaggaagagggagctc

SEQ ID NO: 207 - DNA, 484 bp, synthetic construct
  misc_feature 1..484: note = EnhancerLAP2.31
  source 1..484: mol_type = other DNA
ccacctggggacatgggcgagagggagggacaaggcaagctgaaaggaaggacagccaggggctggccctgggctgacctatccccggtccgcgctccgcccacccatcacagcagccgcggacgctgcgcgccggagcggtccatctcgccagccagccaaccagccgagccgcccagccgacccgagagccccgagagccagactccctcagccatagaagacaccgggcgggagagacggactgaaaaaatatatctttttttattttgtctgggcctggagacccgcagcaggagcggaggtgggtgcggggccgggagccggagcaggaccgggaacaggaacaggaacaggaacaggaacaggaacaggagtggggccgggagcaggagcaggagcgggagccgaagtgggggcaggagcggcggcggccgcagcagcaacagggtcgccccagtccgcggcgaggaagagggagctc

SEQ ID NO: 208 - DNA, 494 bp, synthetic construct
  misc_feature 1..494: note = EnhancerLAP2.32
  source 1..494: mol_type = other DNA
acattctgaaaggttcaaaatagtgttaaagcagaagtatataatactttactgtcattaaatgcacacccaaggacagcaaagaatgttatccccggtccgcgctccgcccacccatcacagcagccgcggacgctgcgcgccggagcggtccatctcgccagccagccaaccagccgagccgcccagccgacccgagagccccgagagccagactccctcagccatagaagacaccgggcgggagagacggactgaaaaaatatatctttttttattttgtctgggcctggagacccgcagcaggagcggaggtgggtgcggggccgggagccggagcaggaccgggaacaggaacaggaacaggaacaggaacaggaacaggagtggggccgggagcaggagcaggagcgggagccgaagtgggggcaggagcggcggcggccgcagcagcaacagggtcgccccagtccgcggcgaggaagagggagctc

SEQ ID NO: 209 - DNA, 494 bp, synthetic construct; identical to SEQ ID NO: 208
  misc_feature 1..494: note = EnhancerLAP2.33
  source 1..494: mol_type = other DNA

SEQ ID NO: 210 - DNA, 504 bp, synthetic construct
  misc_feature 1..504: note = EnhancerLAP2.34
  source 1..504: mol_type = other DNA
ctcagcatttttttacctggctcaatagccggggactccacagaagcagagagagagagagagagagagagagagagagagagagagagagagagagacgatccccggtccgcgctccgcccacccatcacagcagccgcggacgctgcgcgccggagcggtccatctcgccagccagccaaccagccgagccgcccagccgacccgagagccccgagagccagactccctcagccatagaagacaccgggcgggagagacggactgaaaaaatatatctttttttattttgtctgggcctggagacccgcagcaggagcggaggtgggtgcggggccgggagccggagcaggaccgggaacaggaacaggaacaggaacaggaacaggaacaggagtggggccgggagcaggagcaggagcgggagccgaagtgggggcaggagcggcggcggccgcagcagcaacagggtcgccccagtccgcggcgaggaagagggagctc

SEQ ID NO: 211 - DNA, 474 bp, synthetic construct
  misc_feature 1..474: note = EnhancerLAP2.35
  source 1..474: mol_type = other DNA
tctgtgggaaggcaggaagagttgaggctatgaccacagatagaaaacagcagggccacttagccccaacatccccggtccgcgctccgcccacccatcacagcagccgcggacgctgcgcgccggagcggtccatctcgccagccagccaaccagccgagccgcccagccgacccgagagccccgagagccagactccctcagccatagaagacaccgggcgggagagacggactgaaaaaatatatctttttttattttgtctgggcctggagacccgcagcaggagcggaggtgggtgcggggccgggagccggagcaggaccgggaacaggaacaggaacaggaacaggaacaggaacaggagtggggccgggagcaggagcaggagcgggagccgaagtgggggcaggagcggcggcggccgcagcagcaacagggtcgccccagtccgcggcgaggaagagggagctc

SEQ ID NO: 212 - DNA, 504 bp, synthetic construct
  misc_feature 1..504: note = EnhancerLAP2.36
  source 1..504: mol_type = other DNA
tgaagcttgggtggagccacttcgcacaggttgggatgaggaccctgatttgtgtaggtcagggccaagctctcctggtgctcagctcacatggctccttatccccggtccgcgctccgcccacccatcacagcagccgcggacgctgcgcgccggagcggtccatctcgccagccagccaaccagccgagccgcccagccgacccgagagccccgagagccagactccctcagccatagaagacaccgggcgggagagacggactgaaaaaatatatctttttttattttgtctgggcctggagacccgcagcaggagcggaggtgggtgcggggccgggagccggagcaggaccgggaacaggaacaggaacaggaacaggaacaggaacaggagtggggccgggagcaggagcaggagcgggagccgaagtgggggcaggagcggcggcggccgcagcagcaacagggtcgccccagtccgcggcgaggaagagggagctc

SEQ ID NO: 213 - DNA, 494 bp, synthetic construct
  misc_feature 1..494: note = EnhancerLAP2.37
  source 1..494: mol_type = other DNA
cttccaggtgctgggggaaagatttcagacgcatttctgggtgtggctggaccagggcagggggtgggataggactggagggacagacggatccccggtccgcgctccgcccacccatcacagcagccgcggacgctgcgcgccggagcggtccatctcgccagccagccaaccagccgagccgcccagccgacccgagagccccgagagccagactccctcagccatagaagacaccgggcgggagagacggactgaaaaaatatatctttttttattttgtctgggcctggagacccgcagcaggagcggaggtgggtgcggggccgggagccggagcaggaccgggaacaggaacaggaacaggaacaggaacaggaacaggagtggggccgggagcaggagcaggagcgggagccgaagtgggggcaggagcggcggcggccgcagcagcaacagggtcgccccagtccgcggcgaggaagagggagctc

SEQ ID NO: 214 - DNA, 504 bp, synthetic construct
  misc_feature 1..504: note = EnhancerLAP2.38
  source 1..504: mol_type = other DNA
cttaaaaaaaaaaacccactatgttccatctgagaactaagcaaattattctctcctctgggtcctatatatagccacaggcagctacaatagacctataatccccggtccgcgctccgcccacccatcacagcagccgcggacgctgcgcgccggagcggtccatctcgccagccagccaaccagccgagccgcccagccgacccgagagccccgagagccagactccctcagccatagaagacaccgggcgggagagacggactgaaaaaatatatctttttttattttgtctgggcctggagacccgcagcaggagcggaggtgggtgcggggccgggagccggagcaggaccgggaacaggaacaggaacaggaacaggaacaggaacaggagtggggccgggagcaggagcaggagcgggagccgaagtgggggcaggagcggcggcggccgcagcagcaacagggtcgccccagtccgcggcgaggaagagggagctc

SEQ ID NO: 215 - DNA, 484 bp, synthetic construct
  misc_feature 1..484: note = EnhancerLAP2.39
  source 1..484: mol_type = other DNA
ctatcagttaacttgtgcagctaccctgatgtgttctggagactctgtttagcgtatggccttttaaactgtctctttctatccccggtccgcgctccgcccacccatcacagcagccgcggacgctgcgcgccggagcggtccatctcgccagccagccaaccagccgagccgcccagccgacccgagagccccgagagccagactccctcagccatagaagacaccgggcgggagagacggactgaaaaaatatatctttttttattttgtctgggcctggagacccgcagcaggagcggaggtgggtgcggggccgggagccggagcaggaccgggaacaggaacaggaacaggaacaggaacaggaacaggagtggggccgggagcaggagcaggagcgggagccgaagtgggggcaggagcggcggcggccgcagcagcaacagggtcgccccagtccgcggcgaggaagagggagctc

SEQ ID NO: 216 - DNA, 494 bp, synthetic construct
  misc_feature 1..494: note = EnhancerLAP2.40
  source 1..494: mol_type = other DNA
acagaagcctttccgggactgccgaggcttatctgaacttaagaatcgttggaatgttggattacttcagagtactctgttttttcatttatccccggtccgcgctccgcccacccatcacagcagccgcggacgctgcgcgccggagcggtccatctcgccagccagccaaccagccgagccgcccagccgacccgagagccccgagagccagactccctcagccatagaagacaccgggcgggagagacggactgaaaaaatatatctttttttattttgtctgggcctggagacccgcagcaggagcggaggtgggtgcggggccgggagccggagcaggaccgggaacaggaacaggaacaggaacaggaacaggaacaggagtggggccgggagcaggagcaggagcgggagccgaagtgggggcaggagcggcggcggccgcagcagcaacagggtcgccccagtccgcggcgaggaagagggagctc

SEQ ID NO: 217 - DNA, 484 bp, synthetic construct
  misc_feature 1..484: note = EnhancerLAP2.41
  source 1..484: mol_type = other DNA
ccgtaagtgtcccgggggtgggcggggcgcgggccctgcaggctctggaggggacagcacgggagtggcccgcccctggaatccccggtccgcgctccgcccacccatcacagcagccgcggacgctgcgcgccggagcggtccatctcgccagccagccaaccagccgagccgcccagccgacccgagagccccgagagccagactccctcagccatagaagacaccgggcgggagagacggactgaaaaaatatatctttttttattttgtctgggcctggagacccgcagcaggagcggaggtgggtgcggggccgggagccggagcaggaccgggaacaggaacaggaacaggaacaggaacaggaacaggagtggggccgggagcaggagcaggagcgggagccgaagtgggggcaggagcggcggcggccgcagcagcaacagggtcgccccagtccgcggcgaggaagagggagctc

SEQ ID NO: 218 - DNA, 504 bp, synthetic construct
  misc_feature 1..504: note = EnhancerLAP2.42
  source 1..504: mol_type = other DNA
catctttactgtgggaagaggaggcgttggatggctttaaagcggaccctgaatgcattcgatttttatttatttattgtaaggaaataaaattgacctaatccccggtccgcgctccgcccacccatcacagcagccgcggacgctgcgcgccggagcggtccatctcgccagccagccaaccagccgagccgcccagccgacccgagagccccgagagccagactccctcagccatagaagacaccgggcgggagagacggactgaaaaaatatatctttttttattttgtctgggcctggagacccgcagcaggagcggaggtgggtgcggggccgggagccggagcaggaccgggaacaggaacaggaacaggaacaggaacaggaacaggagtggggccgggagcaggagcaggagcgggagccgaagtgggggcaggagcggcggcggccgcagcagcaacagggtcgccccagtccgcggcgaggaagagggagctc

SEQ ID NO: 219 - DNA, 464 bp, synthetic construct
  misc_feature 1..464: note = EnhancerLAP2.43
  source 1..464: mol_type = other DNA
agtcccgccgcgcgccccacggccccgcgggagacgacctcgcaggcaggccgccgcgcaatccccggtccgcgctccgcccacccatcacagcagccgcggacgctgcgcgccggagcggtccatctcgccagccagccaaccagccgagccgcccagccgacccgagagccccgagagccagactccctcagccatagaagacaccgggcgggagagacggactgaaaaaatatatctttttttattttgtctgggcctggagacccgcagcaggagcggaggtgggtgcggggccgggagccggagcaggaccgggaacaggaacaggaacaggaacaggaacaggaacaggagtggggccgggagcaggagcaggagcgggagccgaagtgggggcaggagcggcggcggccgcagcagcaacagggtcgccccagtccgcggcgaggaagagggagctc

SEQ ID NO: 220 - DNA, 504 bp, synthetic construct
  misc_feature 1..504: note = EnhancerLAP2.44
  source 1..504: mol_type = other DNA
taacacccaaggccccagttctgagtggctccttggattcccaggggcaatcaagcttcataccctacatcctcaagaatcctagttaggcacataacttatccccggtccgcgctccgcccacccatcacagcagccgcggacgctgcgcgccggagcggtccatctcgccagccagccaaccagccgagccgcccagccgacccgagagccccgagagccagactccctcagccatagaagacaccgggcgggagagacggactgaaaaaatatatctttttttattttgtctgggcctggagacccgcagcaggagcggaggtgggtgcggggccgggagccggagcaggaccgggaacaggaacaggaacaggaacaggaacaggaacaggagtggggccgggagcaggagcaggagcgggagccgaagtgggggcaggagcggcggcggccgcagcagcaacagggtcgccccagtccgcggcgaggaagagggagctc

SEQ ID NO: 221 - DNA, 504 bp, synthetic construct
  misc_feature 1..504: note = EnhancerLAP2.45
  source 1..504: mol_type = other DNA
ggtaggtcccctccgcccttgagggcacgggcctcagccccggccccacccccgcggcccgtggccccggccccagctcgcagtcgagaggggtgacgggatccccggtccgcgctccgcccacccatcacagcagccgcggacgctgcgcgccggagcggtccatctcgccagccagccaaccagccgagccgcccagccgacccgagagccccgagagccagactccctcagccatagaagacaccgggcgggagagacggactgaaaaaatatatctttttttattttgtctgggcctggagacccgcagcaggagcggaggtgggtgcggggccgggagccggagcaggaccgggaacaggaacaggaacaggaacaggaacaggaacaggagtggggccgggagcaggagcaggagcgggagccgaagtgggggcaggagcggcggcggccgcagcagcaacagggtcgccccagtccgcggcgaggaagagggagctc

SEQ ID NO: 222 - DNA, 494 bp, synthetic construct
  misc_feature 1..494: note = EnhancerLAP2.46
  source 1..494: mol_type = other DNA
atctgccctaaaaaaacaacctcgttgaggtacaggcgcaaggctgcacgccggtgacctgatgctgggcgaccagcgccttcccgccagatccccggtccgcgctccgcccacccatcacagcagccgcggacgctgcgcgccggagcggtccatctcgccagccagccaaccagccgagccgcccagccgacccgagagccccgagagccagactccctcagccatagaagacaccgggcgggagagacggactgaaaaaatatatctttttttattttgtctgggcctggagacccgcagcaggagcggaggtgggtgcggggccgggagccggagcaggaccgggaacaggaacaggaacaggaacaggaacaggaacaggagtggggccgggagcaggagcaggagcgggagccgaagtgggggcaggagcggcggcggccgcagcagcaacagggtcgccccagtccgcggcgaggaagagggagctc

SEQ ID NO: 223 - DNA, 474 bp, synthetic construct
  misc_feature 1..474: note = EnhancerLAP2.47
  source 1..474: mol_type = other DNA
aaaggttctggcttgcgtttgcttttcaaatgacgtcacggaaaggcggagtaagccagtgcgcatgcacatccccggtccgcgctccgcccacccatcacagcagccgcggacgctgcgcgccggagcggtccatctcgccagccagccaaccagccgagccgcccagccgacccgagagccccgagagccagactccctcagccatagaagacaccgggcgggagagacggactgaaaaaatatatctttttttattttgtctgggcctggagacccgcagcaggagcggaggtgggtgcggggccgggagccggagcaggaccgggaacaggaacaggaacaggaacaggaacaggaacaggagtggggccgggagcaggagcaggagcgggagccgaagtgggggcaggagcggcggcggccgcagcagcaacagggtcgccccagtccgcggcgaggaagagggagctc

SEQ ID NO: 224 - DNA, 504 bp, synthetic construct
  misc_feature 1..504: note = EnhancerLAP2.48
  source 1..504: mol_type = other DNA
agtgaggtgagttctgcgccctgggtgcctctgtcacagagttctgccgccgacgtcagtggcccgcagcttactgtctgtgctcttctctgtccccgcaatccccggtccgcgctccgcccacccatcacagcagccgcggacgctgcgcgccggagcggtccatctcgccagccagccaaccagccgagccgcccagccgacccgagagccccgagagccagactccctcagccatagaagacaccgggcgggagagacggactgaaaaaatatatctttttttattttgtctgggcctggagacccgcagcaggagcggaggtgggtgcggggccgggagccggagcaggaccgggaacaggaacaggaacaggaacaggaacaggaacaggagtggggccgggagcaggagcaggagcgggagccgaagtgggggcaggagcggcggcggccgcagcagcaacagggtcgccccagtccgcggcgaggaagagggagctc

SEQ ID NO: 225 - DNA, 484 bp, synthetic construct
  misc_feature 1..484: note = EnhancerLAP2.49
  source 1..484: mol_type = other DNA
cctggggagacaggagagacttgagactgttttccctccctgtgcccgctttctatccctccttccattccccgtggcccatccccggtccgcgctccgcccacccatcacagcagccgcggacgctgcgcgccggagcggtccatctcgccagccagccaaccagccgagccgcccagccgacccgagagccccgagagccagactccctcagccatagaagacaccgggcgggagagacggactgaaaaaatatatctttttttattttgtctgggcctggagacccgcagcaggagcggaggtgggtgcggggccgggagccggagcaggaccgggaacaggaacaggaacaggaacaggaacaggaacaggagtggggccgggagcaggagcaggagcgggagccgaagtgggggcaggagcggcggcggccgcagcagcaacagggtcgccccagtccgcggcgaggaagagggagctc

SEQ ID NO: 226 - DNA, 464 bp, synthetic construct
  misc_feature 1..464: note = EnhancerLAP2.50
  source 1..464: mol_type = other DNA
tcctcttcacccggcaccgtgcgcccccccccccgcaacccccccaagacagcaccgccgatccccggtccgcgctccgcccacccatcacagcagccgcggacgctgcgcgccggagcggtccatctcgccagccagccaaccagccgagccgcccagccgacccgagagccccgagagccagactccctcagccatagaagacaccgggcgggagagacggactgaaaaaatatatctttttttattttgtctgggcctggagacccgcagcaggagcggaggtgggtgcggggccgggagccggagcaggaccgggaacaggaacaggaacaggaacaggaacaggaacaggagtggggccgggagcaggagcaggagcgggagccgaagtgggggcaggagcggcggcggccgcagcagcaacagggtcgccccagtccgcggcgaggaagagggagctc

SEQ ID NO: 227 - DNA, 474 bp, synthetic construct
  misc_feature 1..474: note = EnhancerLAP2.51
  source 1..474: mol_type = other DNA
tggtgagtggcagaggcaggtgctgggcctcccaggggagtcagggtagaatccaccagcccttctccctatccccggtccgcgctccgcccacccatcacagcagccgcggacgctgcgcgccggagcggtccatctcgccagccagccaaccagccgagccgcccagccgacccgagagccccgagagccagactccctcagccatagaagacaccgggcgggagagacggactgaaaaaatatatctttttttattttgtctgggcctggagacccgcagcaggagcggaggtgggtgcggggccgggagccggagcaggaccgggaacaggaacaggaacaggaacaggaacaggaacaggagtggggccgggagcaggagcaggagcgggagccgaagtgggggcaggagcggcggcggccgcagcagcaacagggtcgccccagtccgcggcgaggaagagggagctc

SEQ ID NO: 228 - DNA, 494 bp, synthetic construct
  misc_feature 1..494: note = EnhancerLAP2.52
  source 1..494: mol_type = other DNA
ggcctgcacctgcgcagaggggaagggagttgagtccggccgtgcgcttcggggtgcaccctgaaggtccccctcccccctgcacctttcatccccggtccgcgctccgcccacccatcacagcagccgcggacgctgcgcgccggagcggtccatctcgccagccagccaaccagccgagccgcccagccgacccgagagccccgagagccagactccctcagccatagaagacaccgggcgggagagacggactgaaaaaatatatctttttttattttgtctgggcctggagacccgcagcaggagcggaggtgggtgcggggccgggagccggagcaggaccgggaacaggaacaggaacaggaacaggaacaggaacaggagtggggccgggagcaggagcaggagcgggagccgaagtgggggcaggagcggcggcggccgcagcagcaacagggtcgccccagtccgcggcgaggaagagggagctc

SEQ ID NO: 229 - DNA, 504 bp, synthetic construct
  misc_feature 1..504: note = EnhancerLAP2.53
  source 1..504: mol_type = other DNA
agcttgcaggttggtagactcggcattgggggaggggtacgggggtcgcagggacactgggggttcacagccaccccaaatccttaggcagggtgtgcagatccccggtccgcgctccgcccacccatcacagcagccgcggacgctgcgcgccggagcggtccatctcgccagccagccaaccagccgagccgcccagccgacccgagagccccgagagccagactccctcagccatagaagacaccgggcgggagagacggactgaaaaaatatatctttttttattttgtctgggcctggagacccgcagcaggagcggaggtgggtgcggggccgggagccggagcaggaccgggaacaggaacaggaacaggaacaggaacaggaacaggagtggggccgggagcaggagcaggagcgggagccgaagtgggggcaggagcggcggcggccgcagcagcaacagggtcgccccagtccgcggcgaggaagagggagctc

SEQ ID NO: 230 - DNA, 504 bp, synthetic construct
  misc_feature 1..504: note = EnhancerLAP2.54
  source 1..504: mol_type = other DNA
atttggcctgcaatgggagatttaaattgtcagaagtgaggcaacagagatggcggctgggtccagcacacctctcccctttggggacctcggagtagccatccccggtccgcgctccgcccacccatcacagcagccgcggacgctgcgcgccggagcggtccatctcgccagccagccaaccagccgagccgcccagccgacccgagagccccgagagccagactccctcagccatagaagacaccgggcgggagagacggactgaaaaaatatatctttttttattttgtctgggcctggagacccgcagcaggagcggaggtgggtgcggggccgggagccggagcaggaccgggaacaggaacaggaacaggaacaggaacaggaacaggagtggggccgggagcaggagcaggagcgggagccgaagtgggggcaggagcggcggcggccgcagcagcaacagggtcgccccagtccgcggcgaggaagagggagctc

SEQ ID NO: 231 - DNA, 484 bp, synthetic construct
  misc_feature 1..484: note = EnhancerLAP2.55
  source 1..484: mol_type = other DNA
acactgcagcagagagcacagtgcatcaggcacacatgggacacaaagagccacccccaggccttgcatgccaccccgtgatccccggtccgcgctccgcccacccatcacagcagccgcggacgctgcgcgccggagcggtccatctcgccagccagccaaccagccgagccgcccagccgacccgagagccccgagagccagactccctcagccatagaagacaccgggcgggagagacggactgaaaaaatatatctttttttattttgtctgggcctggagacccgcagcaggagcggaggtgggtgcggggccgggagccggagcaggaccgggaacaggaacaggaacaggaacaggaacaggaacaggagtggggccgggagcaggagcaggagcgggagccgaagtgggggcaggagcggcggcggccgcagcagcaacagggtcgccccagtccgcggcgaggaagagggagctc

SEQ ID NO: 232 - DNA, 494 bp, synthetic construct
  misc_feature 1..494: note = EnhancerLAP2.56
  source 1..494: mol_type = other DNA
tttgctgaaatagcaaggcaagtaagttcgctcagcaagtttcttactttcaatgtttttttgtttgcagtctatagatttttgtataaaatccccggtccgcgctccgcccacccatcacagcagccgcggacgctgcgcgccggagcggtccatctcgccagccagccaaccagccgagccgcccagccgacccgagagccccgagagccagactccctcagccatagaagacaccgggcgggagagacggactgaaaaaatatatctttttttattttgtctgggcctggagacccgcagcaggagcggaggtgggtgcggggccgggagccggagcaggaccgggaacaggaacaggaacaggaacaggaacaggaacaggagtggggccgggagcaggagcaggagcgggagccgaagtgggggcaggagcggcggcggccgcagcagcaacagggtcgccccagtccgcggcgaggaagagggagctc

SEQ ID NO: 233 - DNA, 464 bp, synthetic construct
  misc_feature 1..464: note = EnhancerLAP2.57
  source 1..464: mol_type = other DNA
actaaagcaattaccccaaacggggggtggaacctgccacctccctctcccaggtgtttaatccccggtccgcgctccgcccacccatcacagcagccgcggacgctgcgcgccggagcggtccatctcgccagccagccaaccagccgagccgcccagccgacccgagagccccgagagccagactccctcagccatagaagacaccgggcgggagagacggactgaaaaaatatatctttttttattttgtctgggcctggagacccgcagcaggagcggaggtgggtgcggggccgggagccggagcaggaccgggaacaggaacaggaacaggaacaggaacaggaacaggagtggggccgggagcaggagcaggagcgggagccgaagtgggggcaggagcggcggcggccgcagcagcaacagggtcgccccagtccgcggcgaggaagagggagctc

SEQ ID NO: 234 - DNA, 504 bp, synthetic construct
  misc_feature 1..504: note = EnhancerLAP2.58
  source 1..504: mol_type = other DNA
ttttcccaagaaccacttaacccacgacaacggtgggtttccccttcaaaaggaagatttccactctcccatgatgcacaccaacccctgggcaattctaatccccggtccgcgctccgcccacccatcacagcagccgcggacgctgcgcgccggagcggtccatctcgccagccagccaaccagccgagccgcccagccgacccgagagccccgagagccagactccctcagccatagaagacaccgggcgggagagacggactgaaaaaatatatctttttttattttgtctgggcctggagacccgcagcaggagcggaggtgggtgcggggccgggagccggagcaggaccgggaacaggaacaggaacaggaacaggaacaggaacaggagtggggccgggagcaggagcaggagcgggagccgaagtgggggcaggagcggcggcggccgcagcagcaacagggtcgccccagtccgcggcgaggaagagggagctc

SEQ ID NO: 235 - DNA, 484 bp, synthetic construct
  misc_feature 1..484: note = EnhancerLAP2.59
  source 1..484: mol_type = other DNA
ccagtgagctcttggggcggggtgagagtgcaggggtcttcaacagaatccagaaaccttggtccaagcccttttctctcatccccggtccgcgctccgcccacccatcacagcagccgcggacgctgcgcgccggagcggtccatctcgccagccagccaaccagccgagccgcccagccgacccgagagccccgagagccagactccctcagccatagaagacaccgggcgggagagacggactgaaaaaatatatctttttttattttgtctgggcctggagacccgcagcaggagcggaggtgggtgcggggccgggagccggagcaggaccgggaacaggaacaggaacaggaacaggaacaggaacaggagtggggccgggagcaggagcaggagcgggagccgaagtgggggcaggagcggcggcggccgcagcagcaacagggtcgccccagtccgcggcgaggaagagggagctc

SEQ ID NO: 236 - DNA, 484 bp, synthetic construct
  misc_feature 1..484: note = EnhancerLAP2.60
  source 1..484: mol_type = other DNA
ctaaaaagaaacagaggccgtcaatggccggccacccaagggccagaaccagctctccagcccttccctaccgtgtgtccatccccggtccgcgctccgcccacccatcacagcagccgcggacgctgcgcgccggagcggtccatctcgccagccagccaaccagccgagccgcccagccgacccgagagccccgagagccagactccctcagccatagaagacaccgggcgggagagacggactgaaaaaatatatctttttttattttgtctgggcctggagacccgcagcaggagcggaggtgggtgcggggccgggagccggagcaggaccgggaacaggaacaggaacaggaacaggaacaggaacaggagtggggccgggagcaggagcaggagcgggagccgaagtgggggcaggagcggcggcggccgcagcagcaacagggtcgccccagtccgcggcgaggaagagggagctc

SEQ ID NO: 237 - DNA, 494 bp, synthetic construct
  misc_feature 1..494: note = EnhancerLAP2.61
  source 1..494: mol_type = other DNA
caaggtccgcggccccgggggaggagaagcgcctgctttctgcgcagtcgccccgcccggactggggggcgggacttaggcgacccggggatccccggtccgcgctccgcccacccatcacagcagccgcggacgctgcgcgccggagcggtccatctcgccagccagccaaccagccgagccgcccagccgacccgagagccccgagagccagactccctcagccatagaagacaccgggcgggagagacggactgaaaaaatatatctttttttattttgtctgggcctggagacccgcagcaggagcggaggtgggtgcggggccgggagccggagcaggaccgggaacaggaacaggaacaggaacaggaacaggaacaggagtggggccgggagcaggagcaggagcgggagccgaagtgggggcaggagcggcggcggccgcagcagcaacagggtcgccccagtccgcggcgaggaagagggagctc

SEQ ID NO: 238 - DNA, 504 bp, synthetic construct
  misc_feature 1..504: note = EnhancerLAP2.62
  source 1..504: mol_type = other DNA
gcccaatgtatgatggaggggcccaggcccatcgtggctctggatgcaagggggaggggtgggcccttcagatcactgttgaacacgagggatgttcttgatccccggtccgcgctccgcccacccatcacagcagccgcggacgctgcgcgccggagcggtccatctcgccagccagccaaccagccgagccgcccagccgacccgagagccccgagagccagactccctcagccatagaagacaccgggcgggagagacggactgaaaaaatatatctttttttattttgtctgggcctggagacccgcagcaggagcggaggtgggtgcggggccgggagccggagcaggaccgggaacaggaacaggaacaggaacaggaacaggaacaggagtggggccgggagcaggagcaggagcgggagccgaagtgggggcaggagcggcggcggccgcagcagcaacagggtcgccccagtccgcggcgaggaagagggagctc

SEQ ID NO: 239 - DNA, 504 bp, synthetic construct; identical to SEQ ID NO: 238
  misc_feature 1..504: note = EnhancerLAP2.63
  source 1..504: mol_type = other DNA

SEQ ID NO: 240 - DNA, 504 bp, synthetic construct
  misc_feature 1..504: note = EnhancerLAP2.64
  source 1..504: mol_type = other DNA
gggcagcagcgaccctccccccaacacctttcccgtcccctcccccacggccggcactacctcccccccaccccaggaacaccccccccccgtgcctcccatccccggtccgcgctccgcccacccatcacagcagccgcggacgctgcgcgccggagcggtccatctcgccagccagccaaccagccgagccgcccagccgacccgagagccccgagagccagactccctcagccatagaagacaccgggcgggagagacggactgaaaaaatatatctttttttattttgtctgggcctggagacccgcagcaggagcggaggtgggtgcggggccgggagccggagcaggaccgggaacaggaacaggaacaggaacaggaacaggaacaggagtggggccgggagcaggagcaggagcgggagccgaagtgggggcaggagcggcggcggccgcagcagcaacagggtcgccccagtccgcggcgaggaagagggagctc

SEQ ID NO: 241 - DNA, 504 bp, synthetic construct; identical to SEQ ID NO: 240
  misc_feature 1..504: note = EnhancerLAP2.65
  source 1..504: mol_type = other DNA

SEQ ID NO: 242 - DNA, 494 bp, synthetic construct
  misc_feature 1..494: note = EnhancerLAP2.66
  source 1..494: mol_type = other DNA
cggtgagcgcgctgcaccctcccgggaacccgcgcgcccgtccccgtcccggagctagttgtaatgctttttcctccaaaccctgtttttatccccggtccgcgctccgcccacccatcacagcagccgcggacgctgcgcgccggagcggtccatctcgccagccagccaaccagccgagccgcccagccgacccgagagccccgagagccagactccctcagccatagaagacaccgggcgggagagacggactgaaaaaatatatctttttttattttgtctgggcctggagacccgcagcaggagcggaggtgggtgcggggccgggagccggagcaggaccgggaacaggaacaggaacaggaacaggaacaggaacaggagtggggccgggagcaggagcaggagcgggagccgaagtgggggcaggagcggcggcggccgcagcagcaacagggtcgccccagtccgcggcgaggaagagggagctc

SEQ ID NO: 243 - DNA, 484 bp, synthetic construct
  misc_feature 1..484: note = EnhancerLAP2.67
  source 1..484: mol_type = other DNA
gtagggcccaaccgcacgggcctgggactttgtcgttggggctgagagcccaggccaaattctcatctcccctttccccgatccccggtccgcgctccgcccacccatcacagcagccgcggacgctgcgcgccggagcggtccatctcgccagccagccaaccagccgagccgcccagccgacccgagagccccgagagccagactccctcagccatagaagacaccgggcgggagagacggactgaaaaaatatatctttttttattttgtctgggcctggagacccgcagcaggagcggaggtgggtgcggggccgggagccggagcaggaccgggaacaggaacaggaacaggaacaggaacaggaacaggagtggggccgggagcaggagcaggagcgggagccgaagtgggggcaggagcggcggcggccgcagcagcaacagggtcgccccagtccgcggcgaggaagagggagctc

SEQ ID NO: 244 - DNA, 484 bp, synthetic construct
  misc_feature 1..484: note = EnhancerLAP2.68
  source 1..484: mol_type = other DNA
cacccacactctgcatgctgcatgccacaaatgtgatgtgtgaagaaaaaaagaacagaacgcaagcagaaacgcaaacaatccccggtccgcgctccgcccacccatcacagcagccgcggacgctgcgcgccggagcggtccatctcgccagccagccaaccagccgagccgcccagccgacccgagagccccgagagccagactccctcagccatagaagacaccgggcgggagagacggactgaaaaaatatatctttttttattttgtctgggcctggagacccgcagcaggagcggaggtgggtgcggggccgggagccggagcaggaccgggaacaggaacaggaacaggaacaggaacaggaacaggagtggggccgggagcaggagcaggagcgggagccgaagtgggggcaggagcggcggcggccgcagcagcaacagggtcgccccagtccgcggcgaggaagagggagctc

SEQ ID NO: 245 - DNA, 494 bp, synthetic construct
  misc_feature 1..494: note = EnhancerLAP2.69
  source 1..494: mol_type = other DNA
ctagaaacaccaaagaaacgcttgaatgtcaggaaatgaggcacaacacaaaatgactcgtcacgcccaaggggaggcaaggtcccggctatccccggtccgcgctccgcccacccatcacagcagccgcggacgctgcgcgccggagcggtccatctcgccagccagccaaccagccgagccgcccagccgacccgagagccccgagagccagactccctcagccatagaagacaccgggcgggagagacggactgaaaaaatatatctttttttattttgtctgggcctggagacccgcagcaggagcggaggtgggtgcggggccgggagccggagcaggaccgggaacaggaacaggaacaggaacaggaacaggaacaggagtggggccgggagcaggagcaggagcgggagccgaagtgggggcaggagcggcggcggccgcagcagcaacagggtcgccccagtccgcggcgaggaagagggagctc

SEQ ID NO: 246 - DNA, 504 bp, synthetic construct
  misc_feature 1..504: note = EnhancerLAP2.70
  source 1..504: mol_type = other DNA
catgacgccctgcgagagaaaaggccgcggtcagactcgccccacccaccgcgcgacccgggcccagagagaacacaagggaggccactcctctcaactaatccccggtccgcgctccgcccacccatcacagcagccgcggacgctgcgcgccggagcggtccatctcgccagccagccaaccagccgagccgcccagccgacccgagagccccgagagccagactccctcagccatagaagacaccgggcgggagagacggactgaaaaaatatatctttttttattttgtctgggcctggagacccgcagcaggagcggaggtgggtgcggggccgggagccggagcaggaccgggaacaggaacaggaacaggaacaggaacaggaacaggagtggggccgggagcaggagcaggagcgggagccgaagtgggggcaggagcggcggcggccgcagcagcaacagggtcgccccagtccgcggcgaggaagagggagctc

SEQ ID NO: 247 - DNA, 494 bp, synthetic construct
  misc_feature 1..494: note = EnhancerLAP2.71
  source 1..494: mol_type = other DNA
ggctgggtgagtgctcccccctttctcccgcagtgctttgcgcgcggccggtgggcgggggagggaggggaggggaggcggcggcggcagatccccggtccgcgctccgcccacccatcacagcagccgcggacgctgcgcgccggagcggtccatctcgccagccagccaaccagccgagccgcccagccgacccgagagccccgagagccagactccctcagccatagaagacaccgggcgggagagacggactgaaaaaatatatctttttttattttgtctgggcctggagacccgcagcaggagcggaggtgggtgcggggccgggagccggagcaggaccgggaacaggaacaggaacaggaacaggaacaggaacaggagtggggccgggagcaggagcaggagcgggagccgaagtgggggcaggagcggcggcggccgcagcagcaacagggtcgccccagtccgcggcgaggaagagggagctc

SEQ ID NO: 248 - DNA, 504 bp, synthetic construct
  misc_feature 1..504: note = EnhancerLAP2.72
  source 1..504: mol_type = other DNA
gaaggtaccaaactggccttggtctaaggtctctggactgagggctgtgtgagcacatgcctcagggaagcttggggcactgctgagtgacctggattccatccccggtccgcgctccgcccacccatcacagcagccgcggacgctgcgcgccggagcggtccatctcgccagccagccaaccagccgagccgcccagccgacccgagagccccgagagccagactccctcagccatagaagacaccgggcgggagagacggactgaaaaaatatatctttttttattttgtctgggcctggagacccgcagcaggagcggaggtgggtgcggggccgggagccggagcaggaccgggaacaggaacaggaacaggaacaggaacaggaacaggagtggggccgggagcaggagcaggagcgggagccgaagtgggggcaggagcggcggcggccgcagcagcaacagggtcgccccagtccgcggcgaggaagagggagctc

SEQ ID NO: 249 - DNA, 484 bp, synthetic construct
  misc_feature 1..484: note = EnhancerLAP2.73
  source 1..484: mol_type = other DNA
ccatggcaggtaggatggtccgcgcaggcggggacggggcagcgggacgcgacctctgcagccagttgctatttcgctgcatccccggtccgcgctccgcccacccatcacagcagccgcggacgctgcgcgccggagcggtccatctcgccagccagccaaccagccgagccgcccagccgacccgagagccccgagagccagactccctcagccatagaagacaccgggcgggagagacggactgaaaaaatatatctttttttattttgtctgggcctggagacccgcagcaggagcggaggtgggtgcggggccgggagccggagcaggaccgggaacaggaacaggaacaggaacaggaacaggaacaggagtggggccgggagcaggagcaggagcgggagccgaagtgggggcaggagcggcggcggccgcagcagcaacagggtcgccccagtccgcggcgaggaagagggagctc

SEQ ID NO: 250 - DNA, 484 bp, synthetic construct; identical to SEQ ID NO: 217
  misc_feature 1..484: note = EnhancerLAP2.74
  source 1..484: mol_type = other DNA

SEQ ID NO: 251 - DNA, 504 bp, synthetic construct
  misc_feature 1..504: note = EnhancerLAP2.75
  source 1..504: mol_type = other DNA
ctgtggaacaaaatggggaggttggcgacccagcagaggtccccacccacagctgtggggcagaccgctcctcactgtgaaatgggcaagggagccttgaatccccggtccgcgctccgcccacccatcacagcagccgcggacgctgcgcgccggagcggtccatctcgccagccagccaaccagccgagccgcccagccgacccgagagccccgagagccagactccctcagccatagaagacaccgggcgggagagacggactgaaaaaatatatctttttttattttgtctgggcctggagacccgcagcaggagcggaggtgggtgcggggccgggagccggagcaggaccgggaacaggaacaggaacaggaacaggaacaggaacaggagtggggccgggagcaggagcaggagcgggagccgaagtgggggcaggagcggcggcggccgcagcagcaacagggtcgccccagtccgcggcgaggaagagggagctc

SEQ ID NO: 252 - DNA, 494 bp, synthetic construct
  misc_feature 1..494: note = EnhancerLAP2.76
  source 1..494: mol_type = other DNA
gtgaggcttagagggccatctccccatccatgggtctttctgtgattttcgatggatttgggagcctcaactgagatgcctgtctccctaatccccggtccgcgctccgcccacccatcacagcagccgcggacgctgcgcgccggagcggtccatctcgccagccagccaaccagccgagccgcccagccgacccgagagccccgagagccagactccctcagccatagaagacaccgggcgggagagacggactgaaaaaatatatctttttttattttgtctgggcctggagacccgcagcaggagcggaggtgggtgcggggccgggagccggagcaggaccgggaacaggaacaggaacaggaacaggaacaggaacaggagtggggccgggagcaggagcaggagcgggagccgaagtgggggcaggagcggcggcggccgcagcagcaacagggtcgccccagtccgcggcgaggaagagggagctc

SEQ ID NO: 253 - DNA, 474 bp, synthetic construct
  misc_feature 1..474: note = EnhancerLAP2.77
  source 1..474: mol_type = other DNA
cctgcttggactccagtggcccaattcacttcatccaggcactgcaacccagagaggaaggcagggagacatccccggtccgcgctccgcccacccatcacagcagccgcggacgctgcgcgccggagcggtccatctcgccagccagccaaccagccgagccgcccagccgacccgagagccccgagagccagactccctcagccatagaagacaccgggcgggagagacggactgaaaaaatatatctttttttattttgtctgggcctggagacccgcagcaggagcggaggtgggtgcggggccgggagccggagcaggaccgggaacaggaacaggaacaggaacaggaacaggaacaggagtggggccgggagcaggagcaggagcgggagccgaagtgggggcaggagcggcggcggccgcagcagcaacagggtcgccccagtccgcggcgaggaagagggagctc

SEQ ID NO: 254 - DNA, 464 bp, synthetic construct
  misc_feature 1..464: note = EnhancerLAP2.78
  source 1..464: mol_type = other DNA
ggaaggtaaatcctggcctgggcgcggggcccaccaggactgtcctgcccgcccgtgcccatccccggtccgcgctccgcccacccatcacagcagccgcggacgctgcgcgccggagcggtccatctcgccagccagccaaccagccgagccgcccagccgacccgagagccccgagagccagactccctcagccatagaagacaccgggcgggagagacggactgaaaaaatatatctttttttattttgtctgggcctggagacccgcagcaggagcggaggtgggtgcggggccgggagccggagcaggaccgggaacaggaacaggaacaggaacaggaacaggaacaggagtggggccgggagcaggagcaggagcgggagccgaagtgggggcaggagcggcggcggccgcagcagcaacagggtcgccccagtccgcggcgaggaagagggagctc

SEQ ID NO: 255 - DNA, 494 bp, synthetic construct
  misc_feature 1..494: note = EnhancerLAP2.79
  source 1..494: mol_type = other DNA
gtaccaggcaagaaaacctaccgatttttgatcatatgcctggggtactttactcagtagctctggcctctgcttttctttctctgccttatccccggtccgcgctccgcccacccatcacagcagccgcggacgctgcgcgccggagcggtccatctcgccagccagccaaccagccgagccgcccagccgacccgagagccccgagagccagactccctcagccatagaagacaccgggcgggagagacggactgaaaaaatatatctttttttattttgtctgggcctggagacccgcagcaggagcggaggtgggtgcggggccgggagccggagcaggaccgggaacaggaacaggaacaggaacaggaacaggaacaggagtggggccgggagcaggagcaggagcgggagccgaagtgggggcaggagcggcggcggccgcagcagcaacagggtcgccccagtccgcggcgaggaagagggagctc

SEQ ID NO: 256 - DNA, 484 bp, synthetic construct; identical to SEQ ID NO: 207
  misc_feature 1..484: note = EnhancerLAP2.80
  source 1..484: mol_type = other DNA

SEQ ID NO: 257 - DNA, 504 bp, synthetic construct
  misc_feature 1..504: note = EnhancerLAP2.81
  source 1..504: mol_type = other DNA
gcagtccctgtatcagagaagggaaaaagaattgagtcagctggtgagagctgttatctagcatcccaggactccccagaatcccccacgtgttcactcaatccccggtccgcgctccgcccacccatcacagcagccgcggacgctgcgcgccggagcggtccatctcgccagccagccaaccagccgagccgcccagccgacccgagagccccgagagccagactccctcagccatagaagacaccgggcgggagagacggactgaaaaaatatatctttttttattttgtctgggcctggagacccgcagcaggagcggaggtgggtgcggggccgggagccggagcaggaccgggaacaggaacaggaacaggaacaggaacaggaacaggagtggggccgggagcaggagcaggagcgggagccgaagtgggggcaggagcggcggcggccgcagcagcaacagggtcgccccagtccgcggcgaggaagagggagctc

SEQ ID NO: 258 - DNA, 474 bp, synthetic construct
  misc_feature 1..474: note = EnhancerLAP2.82
  source 1..474: mol_type = other DNA
gccagcacctgcggagaggcggggagtgagcggacaccggatggcgccccagccctcccgcccgcggcccatccccggtccgcgctccgcccacccatcacagcagccgcggacgctgcgcgccggagcggtccatctcgccagccagccaaccagccgagccgcccagccgacccgagagccccgagagccagactccctcagccatagaagacaccgggcgggagagacggactgaaaaaatatatctttttttattttgtctgggcctggagacccgcagcaggagcggaggtgggtgcggggccgggagccggagcaggaccgggaacaggaacaggaacaggaacaggaacaggaacaggagtggggccgggagcaggagcaggagcgggagccgaagtgggggcaggagcggcggcggccgcagcagcaacagggtcgccccagtccgcggcgaggaagagggagctc

SEQ ID NO: 259 - DNA, 484 bp, synthetic construct
  misc_feature 1..484: note = EnhancerLAP2.83
  source 1..484: mol_type = other DNA
gcctaagaaggaagaggacgcagctcaagcggcgtggagtacagcggcacaccgggtagcagctccacagcctaccccatatccccggtccgcgctccgcccacccatcacagcagccgcggacgctgcgcgccggagcggtccatctcgccagccagccaaccagccgagccgcccagccgacccgagagccccgagagccagactccctcagccatagaagacaccgggcgggagagacggactgaaaaaatatatctttttttattttgtctgggcctggagacccgcagcaggagcggaggtgggtgcggggccgggagccggagcaggaccgggaacaggaacaggaacaggaacaggaacaggaacaggagtggggccgggagcaggagcaggagcgggagccgaagtgggggcaggagcggcggcggccgcagcagcaacagggtcgccccagtccgcggcgaggaagagggagctc

SEQ ID NO: 260 - DNA, 484 bp, synthetic construct
  misc_feature 1..484: note = EnhancerLAP2.84
  source 1..484: mol_type = other DNA
ggctgtcgcgttaccccggtaactacgagaggcgcacttcccggaagtgtgggcgcaccacgcggtctcccgcccctccaatccccggtccgcgctccgcccacccatcacagcagccgcggacgctgcgcgccggagcggtccatctcgccagccagccaaccagccgagccgcccagccgacccgagagccccgagagccagactccctcagccatagaagacaccgggcgggagagacggactgaaaaaatatatctttttttattttgtctgggcctggagacccgcagcaggagcggaggtgggtgcggggccgggagccggagcaggaccgggaacaggaacaggaacaggaacaggaacaggaacaggagtggggccgggagcaggagcaggagcgggagccgaagtgggggcaggagcggcggcggccgcagcagcaacagggtcgccccagtccgcggcgaggaagagggagctc

SEQ ID NO: 261 - DNA, 484 bp, synthetic construct
  misc_feature 1..484: note = EnhancerLAP2.85
  source 1..484: mol_type = other DNA
cccctccccgacccccgcgtagttccgtctctcgttcgtagctcaaaccgaggttacgcgcaggcgcgccctgcccgctcatccccggtccgcgctccgcccacccatcacagcagccgcggacgctgcgcgccggagcggtccatctcgccagccagccaaccagccgagccgcccagccgacccgagagccccgagagccagactccctcagccatagaagacaccgggcgggagagacggactgaaaaaatatatctttttttattttgtctgggcctggagacccgcagcaggagcggaggtgggtgcggggccgggagccggagcaggaccgggaacaggaacaggaacaggaacaggaacaggaacaggagtggggccgggagcaggagcaggagcgggagccgaagtgggggcaggagcggcggcggccgcagcagcaacagggtcgccccagtccgcggcgaggaagagggagctc

SEQ ID NO: 262 - DNA, 474 bp, synthetic construct; identical to SEQ ID NO: 223
  misc_feature 1..474: note = EnhancerLAP2.86
  source 1..474: mol_type = other DNA

SEQ ID NO: 263 - DNA, 484 bp, synthetic construct; identical to SEQ ID NO: 225
  misc_feature 1..484: note = EnhancerLAP2.87
  source 1..484: mol_type = other DNA

SEQ ID NO: 264 - DNA, 484 bp, synthetic construct; identical to SEQ ID NO: 217
  misc_feature 1..484: note = EnhancerLAP2.88
  source 1..484: mol_type = other DNA

SEQ ID NO: 265 - DNA, 494 bp, synthetic construct
  misc_feature 1..494: note = EnhancerLAP2.89
  source 1..494: mol_type = other DNA
ggcaggtgagcggtgcgctccgggcggggccggcccggcgggggcggggacgcgggcacgggccggtcccggtgacctctgagccgcttcatccccggtccgcgctccgcccacccatcacagcagccgcggacgctgcgcgccggagcggtccatctcgccagccagccaaccagccgagccgcccagccgacccgagagccccgagagccagactccctcagccatagaagacaccgggcgggagagacggactgaaaaaatatatctttttttattttgtctgggcctggagacccgcagcaggagcggaggtgggtgcggggccgggagccggagcaggaccgggaacaggaacaggaacaggaacaggaacaggaacaggagtggggccgggagcaggagcaggagcgggagccgaagtgggggcaggagcggcggcggccgcagcagcaacagggtcgccccagtccgcggcgaggaagagggagctc

SEQ ID NO: 266 - DNA, 494 bp, synthetic construct; identical to SEQ ID NO: 247
  misc_feature 1..494: note = EnhancerLAP2.90
  source 1..494: mol_type = other DNA

SEQ ID NO: 267 - DNA, 484 bp, synthetic construct
  misc_feature 1..484: note = EnhancerLAP2.91
  source 1..484: mol_type = other DNA
ggtatttgcatctgagagcaatttgtcacacggcgattcgtctgctgggtgggggaggggcggggcggggggcccggtcgatccccggtccgcgctccgcccacccatcacagcagccgcggacgctgcgcgccggagcggtccatctcgccagccagccaaccagccgagccgcccagccgacccgagagccccgagagccagactccctcagccatagaagacaccgggcgggagagacggactgaaaaaatatatctttttttattttgtctgggcctggagacccgcagcaggagcggaggtgggtgcggggccgggagccggagcaggaccgggaacaggaacaggaacaggaacaggaacaggaacaggagtggggccgggagcaggagcaggagcgggagccgaagtgggggcaggagcggcggcggccgcagcagcaacagggtcgccccagtccgcggcgaggaagagggagctc

SEQ ID NO: 268 - DNA, 484 bp, synthetic construct; identical to SEQ ID NO: 261
  misc_feature 1..484: note = EnhancerLAP2.92
  source 1..484: mol_type = other DNA

SEQ ID NO: 269 - DNA, 504 bp, synthetic construct
  misc_feature 1..504: note = EnhancerLAP2.93
  source 1..504: mol_type = other DNA
cgcgaaagcgctggaaacccaaaccccgccctcccgcgggggagggtcagtggcgtgacgtcacacgcgcggcgtaaggggcggggctcgcgcggcctagatccccggtccgcgctccgcccacccatcacagcagccgcggacgctgcgcgccggagcggtccatctcgccagccagccaaccagccgagccgcccagccgacccgagagccccgagagccagactccctcagccatagaagacaccgggcgggagagacggactgaaaaaatatatctttttttattttgtctgggcctggagacccgcagcaggagcggaggtgggtgcggggccgggagccggagcaggaccgggaacaggaacaggaacaggaacaggaacaggaacaggagtggggccgggagcaggagcaggagcgggagccgaagtgggggcaggagcggcggcggccgcagcagcaacagggtcgccccagtccgcggcgaggaagagggagctc

SEQ ID NO: 270 - DNA, 494 bp, synthetic construct
  misc_feature 1..494: note = EnhancerLAP2.94
  source 1..494: mol_type = other DNA
aactaacatcaaagagagagagagaaatcgagattctgtcacctcaccttagatcccagcccaggcccccgcctccaaactggcccctcaatccccggtccgcgctccgcccacccatcacagcagccgcggacgctgcgcgccggagcggtccatctcgccagccagccaaccagccgagccgcccagccgacccgagagccccgagagccagactccctcagccatagaagacaccgggcgggagagacggactgaaaaaatatatctttttttattttgtctgggcctggagacccgcagcaggagcggaggtgggtgcggggccgggagccggagcaggaccgggaacaggaacaggaacaggaacaggaacaggaacaggagtggggccgggagcaggagcaggagcgggagccgaagtgggggcaggagcggcggcggccgcagcagcaacagggtcgccccagtccgcggcgaggaagagggagctc

SEQ ID NO: 271 - DNA, 484 bp, synthetic construct
  misc_feature 1..484: note = EnhancerLAP2.95
  source 1..484: mol_type = other DNA
actctgtggtgagggggaggggagtagtgacagacctctcgcgagacttcagcggcgctgctcacgtgtgccgggaggggatccccggtccgcgctccgcccacccatcacagcagccgcggacgctgcgcgccggagcggtccatctcgccagccagccaaccagccgagccgcccagccgacccgagagccccgagagccagactccctcagccatagaagacaccgggcgggagagacggactgaaaaaatatatctttttttattttgtctgggcctggagacccgcagcaggagcggaggtgggtgcggggccgggagccggagcaggaccgggaacaggaacaggaacaggaacaggaacaggaacaggagtggggccgggagcaggagcaggagcgggagccgaagtgggggcaggagcggcggcggccgcagcagcaacagggtcgccccagtccgcggcgaggaagagggagctc

SEQ ID NO: 272 - DNA, 464 bp, synthetic construct
  misc_feature 1..464: note = EnhancerLAP2.96
  source 1..464: mol_type = other DNA
atgcgcaggcgcggctcagctacccccgcagcgtgggcggaaattaaaaaaaaaaaaaaaatccccggtccgcgctccgcccacccatcacagcagccgcggacgctgcgcgccggagcggtccatctcgccagccagccaaccagccgagccgcccagccgacccgagagccccgagagccagactccctcagccatagaagacaccgggcgggagagacggactgaaaaaatatatctttttttattttgtctgggcctggagacccgcagcaggagcggaggtgggtgcggggccgggagccggagcaggaccgggaacaggaacaggaacaggaacaggaacaggaacaggagtggggccgggagcaggagcaggagcgggagccgaagtgggggcaggagcggcggcggccgcagcagcaacagggtcgccccagtccgcggcgaggaagagggagctc

SEQ ID NO: 273 - DNA, 474 bp, synthetic construct
  misc_feature 1..474: note = EnhancerLAP2.97
  source 1..474: mol_type = other DNA
gtccccggaagagaaagtgaaagtggcagtcccggaagtgctgcagaccgaacggaaaactgtcccctcaatccccggtccgcgctccgcccacccatcacagcagccgcggacgctgcgcgccggagcggtccatctcgccagccagccaaccagccgagccgcccagccgacccgagagccccgagagccagactccctcagccatagaagacaccgggcgggagagacggactgaaaaaatatatctttttttattttgtctgggcctggagacccgcagcaggagcggaggtgggtgcggggccgggagccggagcaggaccgggaacaggaacaggaacaggaacaggaacaggaacaggagtggggccgggagcaggagcaggagcgggagccgaagtgggggcaggagcggcggcggccgcagcagcaacagggtcgccccagtccgcggcgaggaagagggagctc

SEQ ID NO: 274 - DNA, 484 bp, synthetic construct
  misc_feature 1..484: note = EnhancerLAP2.98
  source 1..484: mol_type = other DNA
gctcgatcggtaccgccccgccccaggcccggcccggcccgattcggccgggtgacagcgcctgactcccgctcggctccatccccggtccgcgctccgcccacccatcacagcagccgcggacgctgcgcgccggagcggtccatctcgccagccagccaaccagccgagccgcccagccgacccgagagccccgagagccagactccctcagccatagaagacaccgggcgggagagacggactgaaaaaatatatctttttttattttgtctgggcctggagacccgcagcaggagcggaggtgggtgcggggccgggagccggagcaggaccgggaacaggaacaggaacaggaacaggaacaggaacaggagtggggccgggagcaggagcaggagcgggagccgaagtgggggcaggagcggcggcggccgcagcagcaacagggtcgccccagtccgcggcgaggaagagggagctc

SEQ ID NO: 275 - DNA, 504 bp, synthetic construct
  misc_feature 1..504: note = EnhancerLAP2.99
  source 1..504: mol_type = other DNA
cctgggatggggaaagtacccattacactgcagcttccacctcatcctgctcccttgccttcactaacggcagttcaacacctcctggtcaacttcaccaatccccggtccgcgctccgcccacccatcacagcagccgcggacgctgcgcgccggagcggtccatctcgccagccagccaaccagccgagccgcccagccgacccgagagccccgagagccagactccctcagccatagaagacaccgggcgggagagacggactgaaaaaatatatctttttttattttgtctgggcctggagacccgcagcaggagcggaggtgggtgcggggccgggagccggagcaggaccgggaacaggaacaggaacaggaacaggaacaggaacaggagtggggccgggagcaggagcaggagcgggagccgaagtgggggcaggagcggcggcggccgcagcagcaacagggtcgccccagtccgcggcgaggaagagggagctc

SEQ ID NO: 276 - DNA, 484 bp, synthetic construct
  misc_feature 1..484: note = EnhancerLAP2.100
  source 1..484: mol_type = other DNA
ggtaagtggcttaagtgtagtaggacggccggctaacggcttgtcactgggcagtgctgattcatgcttctgtccttcccatccccggtccgcgctccgcccacccatcacagcagccgcggacgctgcgcgccggagcggtccatctcgccagccagccaaccagccgagccgcccagccgacccgagagccccgagagccagactccctcagccatagaagacaccgggcgggagagacggactgaaaaaatatatctttttttattttgtctgggcctggagacccgcagcaggagcggaggtgggtgcggggccgggagccggagcaggaccgggaacaggaacaggaacaggaacaggaacaggaacaggagtggggccgggagcaggagcaggagcgggagccgaagtgggggcaggagcggcggcggccgcagcagcaacagggtcgccccagtccgcggcgaggaagagggagctc

SEQ ID NO: 277 - DNA, 494 bp, synthetic construct
  misc_feature 1..494: note = EnhancerLAP2.101
  source 1..494: mol_type = other DNA
tcctcggccccgcccctgctacggtccccgcgccctcctccccctcttccgcttgggtctggtgaggccccgctgcggctgcggacgccgatccccggtccgcgctccgcccacccatcacagcagccgcggacgctgcgcgccggagcggtccatctcgccagccagccaaccagccgagccgcccagccgacccgagagccccgagagccagactccctcagccatagaagacaccgggcgggagagacggactgaaaaaatatatctttttttattttgtctgggcctggagacccgcagcaggagcggaggtgggtgcggggccgggagccggagcaggaccgggaacaggaacaggaacaggaacaggaacaggaacaggagtggggccgggagcaggagcaggagcgggagccgaagtgggggcaggagcggcggcggccgcagcagcaacagggtcgccccagtccgcggcgaggaagagggagctc

SEQ ID NO: 278 - DNA, 484 bp, synthetic construct
  misc_feature 1..484: note = EnhancerLAP2.102
  source 1..484: mol_type = other DNA
tctgggtacatgaaagaatctggttgccgtttgatgcgcttgggaaacccagatcaccaccccacagggcgcccctaagtatccccggtccgcgctccgcccacccatcacagcagccgcggacgctgcgcgccggagcggtccatctcgccagccagccaaccagccgagccgcccagccgacccgagagccccgagagccagactccctcagccatagaagacaccgggcgggagagacggactgaaaaaatatatctttttttattttgtctgggcctggagacccgcagcaggagcggaggtgggtgcggggccgggagccggagcaggaccgggaacaggaacaggaacaggaacaggaacaggaacaggagtggggccgggagcaggagcaggagcgggagccgaagtgggggcaggagcggcggcggccgcagcagcaacagggtcgccccagtccgcggcgaggaagagggagctc

SEQ ID NO: 279 - DNA, 504 bp, synthetic construct
  misc_feature 1..504: note = EnhancerLAP2.103
  source 1..504: mol_type = other DNA
ggcgagagctgcgaggaaagtgagggaaggatgcgcgcgctaagcgggacccacggcgatccgcgtaccgggctcagtcccacctgcccggccgcccggcatccccggtccgcgctccgcccacccatcacagcagccgcggacgctgcgcgccggagcggtccatctcgccagccagccaaccagccgagccgcccagccgacccgagagccccgagagccagactccctcagccatagaagacaccgggcgggagagacggactgaaaaaatatatctttttttattttgtctgggcctggagacccgcagcaggagcggaggtgggtgcggggccgggagccggagcaggaccgggaacaggaacaggaacaggaacaggaacaggaacaggagtggggccgggagcaggagcaggagcgggagccgaagtgggggcaggagcggcggcggccgcagcagcaacagggtcgccccagtccgcggcgaggaagagggagctc

SEQ ID NO: 280 - DNA, 484 bp, synthetic construct
  misc_feature 1..484: note = EnhancerLAP.104
  source 1..484: mol_type = other DNA
gacgtgcgaagcccgagtcacgtggcctcccgggccggcgggggcgggcacatgcggccggccgccggcctcccgaccctatccccggtccgcgctccgcccacccatcacagcagccgcggacgctgcgcgccggagcggtccatctcgccagccagccaaccagccgagccgcccagccgacccgagagccccgagagccagactccctcagccatagaagacaccgggcgggagagacggactgaaaaaatatatctttttttattttgtctgggcctggagacccgcagcaggagcggaggtgggtgcggggccgggagccggagcaggaccgggaacaggaacaggaacaggaacaggaacaggaacaggagtggggccgggagcaggagcaggagcgggagccgaagtgggggcaggagcggcggcggccgcagcagcaacagggtcgccccagtccgcggcgaggaagagggagctc

SEQ ID NO: 281 - DNA, 474 bp, synthetic construct
  misc_feature 1..474: note = EnhancerLAP.105
  source 1..474: mol_type = other DNA
aaaactgtgggtgtacataggtttgttatctaattttgtaataatggggaccaagctcagtctaccagtaatccccggtccgcgctccgcccacccatcacagcagccgcggacgctgcgcgccggagcggtccatctcgccagccagccaaccagccgagccgcccagccgacccgagagccccgagagccagactccctcagccatagaagacaccgggcgggagagacggactgaaaaaatatatctttttttattttgtctgggcctggagacccgcagcaggagcggaggtgggtgcggggccgggagccggagcaggaccgggaacaggaacaggaacaggaacaggaacaggaacaggagtggggccgggagcaggagcaggagcgggagccgaagtgggggcaggagcggcggcggccgcagcagcaacagggtcgccccagtccgcggcgaggaagagggagctc

SEQ ID NO: 282 - DNA, 504 bp, synthetic construct
  misc_feature 1..504: note = EnhancerLAP.106
  source 1..504: mol_type = other DNA
aaatggtaactatagctctcacctgcttcagggaaggaaagacaatttcattcttttttttttttttttttttttttttttttcgagacagggtttctctatccccggtccgcgctccgcccacccatcacagcagccgcggacgctgcgcgccggagcggtccatctcgccagccagccaaccagccgagccgcccagccgacccgagagccccgagagccagactccctcagccatagaagacaccgggcgggagagacggactgaaaaaatatatctttttttattttgtctgggcctggagacccgcagcaggagcggaggtgggtgcggggccgggagccggagcaggaccgggaacaggaacaggaacaggaacaggaacaggaacaggagtggggccgggagcaggagcaggagcgggagccgaagtgggggcaggagcggcggcggccgcagcagcaacagggtcgccccagtccgcggcgaggaagagggagctc

SEQ ID NO: 283 - DNA, 484 bp, synthetic construct; identical to SEQ ID NO: 207
  misc_feature 1..484: note = EnhancerLAP.107
  source 1..484: mol_type = other DNA

SEQ ID NO: 284 - DNA, 474 bp, synthetic construct
  misc_feature 1..474: note = EnhancerLAP.108
  source 1..474: mol_type = other DNA
tgtggttagtatggctccgcgtgcggccgcggttccgtgagcggcacgtgggcctggtgggctggctcggatccccggtccgcgctccgcccacccatcacagcagccgcggacgctgcgcgccggagcggtccatctcgccagccagccaaccagccgagccgcccagccgacccgagagccccgagagccagactccctcagccatagaagacaccgggcgggagagacggactgaaaaaatatatctttttttattttgtctgggcctggagacccgcagcaggagcggaggtgggtgcggggccgggagccggagcaggaccgggaacaggaacaggaacaggaacaggaacaggaacaggagtggggccgggagcaggagcaggagcgggagccgaagtgggggcaggagcggcggcggccgcagcagcaacagggtcgccccagtccgcggcgaggaagagggagctc

SEQ ID NO: 285 - DNA, 484 bp, synthetic construct
  misc_feature 1..484: note = EnhancerLAP.109
  source 1..484: mol_type = other DNA
ccacatttccagcacgcctttctgtcaggatgcctgaccctccgccccgacactccagtaccagccaaacccccaaagctatccccggtccgcgctccgcccacccatcacagcagccgcggacgctgcgcgccggagcggtccatctcgccagccagccaaccagccgagccgcccagccgacccgagagccccgagagccagactccctcagccatagaagacaccgggcgggagagacggactgaaaaaatatatctttttttattttgtctgggcctggagacccgcagcaggagcggaggtgggtgcggggccgggagccggagcaggaccgggaacaggaacaggaacaggaacaggaacaggaacaggagtggggccgggagcaggagcaggagcgggagccgaagtgggggcaggagcggcggcggccgcagcagcaacagggtcgccccagtccgcggcgaggaagagggagctc

SEQ ID NO: 286 - DNA, 504 bp, synthetic construct
  misc_feature 1..504: note = EnhancerLAP2.110
  source 1..504: mol_type = other DNA
gtgagtcatcttttataccacatggagacacacctgaaatggttaaggtttcttgaaatatttcagatgatactcatatttttcttttcttttaattcaaatccccggtccgcgctccgcccacccatcacagcagccgcggacgctgcgcgccggagcggtccatctcgccagccagccaaccagccgagccgcccagccgacccgagagccccgagagccagactccctcagccatagaagacaccgggcgggagagacggactgaaaaaatatatctttttttattttgtctgggcctggagacccgcagcaggagcggaggtgggtgcggggccgggagccggagcaggaccgggaacaggaacaggaacaggaacaggaacaggaacaggagtggggccgggagcaggagcaggagcgggagccgaagtgggggcaggagcggcggcggccgcagcagcaacagggtcgccccagtccgcggcgaggaagagggagctc

SEQ ID NO: 287 - DNA, 494 bp, synthetic construct
  misc_feature 1..494: note = EnhancerLAP2.111
  source 1..494: mol_type = other DNA
actataaaataaatacatttgcattttaatcttaactttaaagcattttagacaaaaggacagcttaggagtcagataaatattcatactatccccggtccgcgctccgcccacccatcacagcagccgcggacgctgcgcgccggagcggtccatctcgccagccagccaaccagccgagccgcccagccgacccgagagccccgagagccagactccctcagccatagaagacaccgggcgggagagacggactgaaaaaatatatctttttttattttgtctgggcctggagacccgcagcaggagcggaggtgggtgcggggccgggagccggagcaggaccgggaacaggaacaggaacaggaacaggaacaggaacaggagtggggccgggagcaggagcaggagcgggagccgaagtgggggcaggagcggcggcggccgcagcagcaacagggtcgccccagtccgcggcgaggaagagggagctc

SEQ ID NO: 288 - DNA, 504 bp, synthetic construct
  misc_feature 1..504: note = EnhancerLAP2.112
  source 1..504: mol_type = other DNA
caattcctacaataattcagaaatagaataaagatcatttaagaaaatgcaggtctcattcttggaaaaatttgaaaaacaaaacaaacaaacaaaaaaaatccccggtccgcgctccgcccacccatcacagcagccgcggacgctgcgcgccggagcggtccatctcgccagccagccaaccagccgagccgcccagccgacccgagagccccgagagccagactccctcagccatagaagacaccgggcgggagagacggactgaaaaaatatatctttttttattttgtctgggcctggagacccgcagcaggagcggaggtgggtgcggggccgggagccggagcaggaccgggaacaggaacaggaacaggaacaggaacaggaacaggagtggggccgggagcaggagcaggagcgggagccgaagtgggggcaggagcggcggcggccgcagcagcaacagggtcgccccagtccgcggcgaggaagagggagctc

SEQ ID NO: 289 - DNA, 494 bp, synthetic construct
  misc_feature 1..494: note = EnhancerLAP2.113
  source 1..494: mol_type = other DNA
cctaggtgaacccgaggcaggagaggaaaggagaatgagcctgggcagtgagtcaacattagttgtgggaaataaatccaggcaaggagtatccccggtccgcgctccgcccacccatcacagcagccgcggacgctgcgcgccggagcggtccatctcgccagccagccaaccagccgagccgcccagccgacccgagagccccgagagccagactccctcagccatagaagacaccgggcgggagagacggactgaaaaaatatatctttttttattttgtctgggcctggagacccgcagcaggagcggaggtgggtgcggggccgggagccggagcaggaccgggaacaggaacaggaacaggaacaggaacaggaacaggagtggggccgggagcaggagcaggagcgggagccgaagtgggggcaggagcggcggcggccgcagcagcaacagggtcgccccagtccgcggcgaggaagagggagctc

SEQ ID NO: 290 - DNA, 484 bp, synthetic construct; identical to SEQ ID NO: 225
  misc_feature 1..484: note = EnhancerLAP2.114
  source 1..484: mol_type = other DNA

SEQ ID NO: 291 - DNA, 464 bp, synthetic construct; identical to SEQ ID NO: 226
  misc_feature 1..464: note = EnhancerLAP2.115
  source 1..464: mol_type = other DNA

SEQ ID NO: 292 - DNA, 494 bp, synthetic construct
  misc_feature 1..494: note = EnhancerLAP2.116
  source 1..494: mol_type = other DNA
ctggttaggagatgggagacgtgacaaggggtgagtgggtggtgtggggtctagaggctgggcctggagagacagcttctctgcaccaccatccccggtccgcgctccgcccacccatcacagcagccgcggacgctgcgcgccggagcggtccatctcgccagccagccaaccagccgagccgcccagccgacccgagagccccgagagccagactccctcagccatagaagacaccgggcgggagagacggactgaaaaaatatatctttttttattttgtctgggcctggagacccgcagcaggagcggaggtgggtgcggggccgggagccggagcaggaccgggaacaggaacaggaacaggaacaggaacaggaacaggagtggggccgggagcaggagcaggagcgggagccgaagtgggggcaggagcggcggcggccgcagcagcaacagggtcgccccagtccgcggcgaggaagagggagctc

SEQ ID NO: 293 - DNA, 504 bp, synthetic construct
  misc_feature 1..504: note = EnhancerLAP2.117
  source 1..504: mol_type = other DNA
atgcctgtagggggcaccacagcccggctctgggccacactgcccgtcctgcctctcctgctatgctgttccctccccactttaggcctgggccctttccatccccggtccgcgctccgcccacccatcacagcagccgcggacgctgcgcgccggagcggtccatctcgccagccagccaaccagccgagccgcccagccgacccgagagccccgagagccagactccctcagccatagaagacaccgggcgggagagacggactgaaaaaatatatctttttttattttgtctgggcctggagacccgcagcaggagcggaggtgggtgcggggccgggagccggagcaggaccgggaacaggaacaggaacaggaacaggaacaggaacaggagtggggccgggagcaggagcaggagcgggagccgaagtgggggcaggagcggcggcggccgcagcagcaacagggtcgccccagtccgcggcgaggaagagggagctc

SEQ ID NO: 294 - DNA, 494 bp, synthetic construct
  misc_feature 1..494: note = EnhancerLAP2.118
  source 1..494: mol_type = other DNA
accctaaggtgagaagacacgccctgagtcctcagagctgggaatagcatcccaaaccctcttccttgaccttgaaccgccacctgctctatccccggtccgcgctccgcccacccatcacagcagccgcggacgctgcgcgccggagcggtccatctcgccagccagccaaccagccgagccgcccagccgacccgagagccccgagagccagactccctcagccatagaagacaccgggcgggagagacggactgaaaaaatatatctttttttattttgtctgggcctggagacccgcagcaggagcggaggtgggtgcggggccgggagccggagcaggaccgggaacaggaacaggaacaggaacaggaacaggaacaggagtggggccgggagcaggagcaggagcgggagccgaagtgggggcaggagcggcggcggccgcagcagcaacagggtcgccccagtccgcggcgaggaagagggagctc

SEQ ID NO: 295 - DNA, 504 bp, synthetic construct
  misc_feature 1..504: note = EnhancerLAP2.119
  source 1..504: mol_type = other DNA
caggtacctgcacgagggtcagaaggcatcacgtgggtgggtgggtggagggtgtgaggaacgaggacaccaatggcaggaaagatcagaagtatcctgtatccccggtccgcgctccgcccacccatcacagcagccgcggacgctgcgcgccggagcggtccatctcgccagccagccaaccagccgagccgcccagccgacccgagagccccgagagccagactccctcagccatagaagacaccgggcgggagagacggactgaaaaaatatatctttttttattttgtctgggcctggagacccgcagcaggagcggaggtgggtgcggggccgggagccggagcaggaccgggaacaggaacaggaacaggaacaggaacaggaacaggagtggggccgggagcaggagcaggagcgggagccgaagtgggggcaggagcggcggcggccgcagcagcaacagggtcgccccagtccgcggcgaggaagagggagctc

SEQ ID NO: 296 - DNA, 474 bp, synthetic construct
  misc_feature 1..474: note = EnhancerLAP2.120
  source 1..474: mol_type = other DNA
ctatgagagtgggttaggggtcatgggtctgccttttatactaccactccctgacacgcccaccggcccaatccccggtccgcgctccgcccacccatcacagcagccgcggacgctgcgcgccggagcggtccatctcgccagccagccaaccagccgagccgcccagccgacccgagagccccgagagccagactccctcagccatagaagacaccgggcgggagagacggactgaaaaaatatatctttttttattttgtctgggcctggagacccgcagcaggagcggaggtgggtgcggggccgggagccggagcaggaccgggaacaggaacaggaacaggaacaggaacaggaacaggagtggggccgggagcaggagcaggagcgggagccgaagtgggggcaggagcggcggcggccgcagcagcaacagggtcgccccagtccgcggcgaggaagagggagctc

SEQ ID NO: 297 - DNA, 504 bp, synthetic construct
  misc_feature 1..504: note = EnhancerLAP2.121
  source 1..504: mol_type = other DNA
actccaatgtgagtctacccttctgccctgccctgggtgggggtggctagcattgacattgtatctcaccaatgcatcctcattcccctgggaccctacaatccccggtccgcgctccgcccacccatcacagcagccgcggacgctgcgcgccggagcggtccatctcgccagccagccaaccagccgagccgcccagccgacccgagagccccgagagccagactccctcagccatagaagacaccgggcgggagagacggactgaaaaaatatatctttttttattttgtctgggcctggagacccgcagcaggagcggaggtgggtgcggggccgggagccggagcaggaccgggaacaggaacaggaacaggaacaggaacaggaacaggagtggggccgggagcaggagcaggagcgggagccgaagtgggggcaggagcggcggcggccgcagcagcaacagggtcgccccagtccgcggcgaggaagagggagctc

SEQ ID NO: 298 - DNA, 504 bp, synthetic construct
  misc_feature 1..504: note = EnhancerLAP2.122
  source 1..504: mol_type = other DNA
ctgcggggaaagggagaaaacaggccggggcggcgggtcagtaacgccgcgggcgctggggattcctgtccagcccggcccggccaatcgcaaatacagaatccccggtccgcgctccgcccacccatcacagcagccgcggacgctgcgcgccggagcggtccatctcgccagccagccaaccagccgagccgcccagccgacccgagagccccgagagccagactccctcagccatagaagacaccgggcgggagagacggactgaaaaaatatatctttttttattttgtctgggcctggagacccgcagcaggagcggaggtgggtgcggggccgggagccggagcaggaccgggaacaggaacaggaacaggaacaggaacaggaacaggagtggggccgggagcaggagcaggagcgggagccgaagtgggggcaggagcggcggcggccgcagcagcaacagggtcgccccagtccgcggcgaggaagagggagctc

SEQ ID NO: 299 - DNA, 474 bp, synthetic construct
  misc_feature 1..474: note = EnhancerLAP2.123
  source 1..474: mol_type = other DNA
aaagacacgtaggtagagcgccgcccccgcctgcgcgccccctggcaccgggccgccccctcgggcccggatccccggtccgcgctccgcccacccatcacagcagccgcggacgctgcgcgccggagcggtccatctcgccagccagccaaccagccgagccgcccagccgacccgagagccccgagagccagactccctcagccatagaagacaccgggcgggagagacggactgaaaaaatatatctttttttattttgtctgggcctggagacccgcagcaggagcggaggtgggtgcggggccgggagccggagcaggaccgggaacaggaacaggaacaggaacaggaacaggaacaggagtggggccgggagcaggagcaggagcgggagccgaagtgggggcaggagcggcggcggccgcagcagcaacagggtcgccccagtccgcggcgaggaagagggagctc

SEQ ID NO: 300 - DNA, 494 bp, synthetic construct
  misc_feature 1..494: note = EnhancerLAP2.124
  source 1..494: mol_type = other DNA
tgagtgagtaggcggggcgtgtgaggacaggggcgggcctcggcgtgaagggggcgtgaccacaagtgggggtgtggtctgaagggatagatccccggtccgcgctccgcccacccatcacagcagccgcggacgctgcgcgccggagcggtccatctcgccagccagccaaccagccgagccgcccagccgacccgagagccccgagagccagactccctcagccatagaagacaccgggcgggagagacggactgaaaaaatatatctttttttattttgtctgggcctggagacccgcagcaggagcggaggtgggtgcggggccgggagccggagcaggaccgggaacaggaacaggaacaggaacaggaacaggaacaggagtggggccgggagcaggagcaggagcgggagccgaagtgggggcaggagcggcggcggccgcagcagcaacagggtcgccccagtccgcggcgaggaagagggagctc

SEQ ID NO: 301 - DNA, 504 bp, synthetic construct
  misc_feature 1..504: note = EnhancerLAP2.125
  source 1..504: mol_type = other DNA
ggtgacgtgtggggcaaggctggggaaggagggatggaaatatggatggtggccccaggaactgtcacttctttctctagaactggggtcacctccccagatccccggtccgcgctccgcccacccatcacagcagccgcggacgctgcgcgccggagcggtccatctcgccagccagccaaccagccgagccgcccagccgacccgagagccccgagagccagactccctcagccatagaagacaccgggcgggagagacggactgaaaaaatatatctttttttattttgtctgggcctggagacccgcagcaggagcggaggtgggtgcggggccgggagccggagcaggaccgggaacaggaacaggaacaggaacaggaacaggaacaggagtggggccgggagcaggagcaggagcgggagccgaagtgggggcaggagcggcggcggccgcagcagcaacagggtcgccccagtccgcggcgaggaagagggagctc

SEQ ID NO: 302 - DNA, 484 bp, synthetic construct; identical to SEQ ID NO: 261
  misc_feature 1..484: note = EnhancerLAP2.126
  source 1..484: mol_type = other DNA

SEQ ID NO: 303 - DNA, 474 bp, synthetic construct
  misc_feature 1..474: note = EnhancerLAP.127
  source 1..474: mol_type = other DNA
gatactcaggtactctgtccactctctcggaagctcctgcttcactggggtttttttgtttgtttgtttgatccccggtccgcgctccgcccacccatcacagcagccgcggacgctgcgcgccggagcggtccatctcgccagccagccaaccagccgagccgcccagccgacccgagagccccgagagccagactccctcagccatagaagacaccgggcgggagagacggactgaaaaaatatatctttttttattttgtctgggcctggagacccgcagcaggagcggaggtgggtgcggggccgggagccggagcaggaccgggaacaggaacaggaacaggaacaggaacaggaacaggagtggggccgggagcaggagcaggagcgggagccgaagtgggggcaggagcggcggcggccgcagcagcaacagggtcgccccagtccgcggcgaggaagagggagctc

SEQ ID NO: 304 - DNA, 474 bp, synthetic construct
  misc_feature 1..474: note = EnhancerLAP2.128
  source 1..474: mol_type = other DNA
ggtgagaggctgcagagggagcggcttccttctcttcccccttccttcctctgactactagggggggtaaatccccggtccgcgctccgcccacccatcacagcagccgcggacgctgcgcgccggagcggtccatctcgccagccagccaaccagccgagccgcccagccgacccgagagccccgagagccagactccctcagccatagaagacaccgggcgggagagacggactgaaaaaatatatctttttttattttgtctgggcctggagacccgcagcaggagcggaggtgggtgcggggccgggagccggagcaggaccgggaacaggaacaggaacaggaacaggaacaggaacaggagtggggccgggagcaggagcaggagcgggagccgaagtgggggcaggagcggcggcggccgcagcagcaacagggtcgccccagtccgcggcgaggaagagggagctc

SEQ ID NO: 305 - DNA, 494 bp, synthetic construct
  misc_feature 1..494: note = EnhancerLAP2.129
  source 1..494: mol_type = other DNA
tctctctctctctctctctctctctctctctctctctctctctgccacccccccctcgttcctctggctggctcggctgaagcgagaaggatccccggtccgcgctccgcccacccatcacagcagccgcggacgctgcgcgccggagcggtccatctcgccagccagccaaccagccgagccgcccagccgacccgagagccccgagagccagactccctcagccatagaagacaccgggcgggagagacggactgaaaaaatatatctttttttattttgtctgggcctggagacccgcagcaggagcggaggtgggtgcggggccgggagccggagcaggaccgggaacaggaacaggaacaggaacaggaacaggaacaggagtggggccgggagcaggagcaggagcgggagccgaagtgggggcaggagcggcggcggccgcagcagcaacagggtcgccccagtccgcggcgaggaagagggagctc

SEQ ID NO: 306 - DNA, 494 bp, synthetic construct
  misc_feature 1..494: note = EnhancerLAP2.130
  source 1..494: mol_type = other DNA
aacaaaggagagccggatgtgtgggatatgaggctgaataaaaatggctgaaaagcaagcgaggctggagggcggccacacccagtgctaatccccggtccgcgctccgcccacccatcacagcagccgcggacgctgcgcgccggagcggtccatctcgccagccagccaaccagccgagccgcccagccgacccgagagccccgagagccagactccctcagccatagaagacaccgggcgggagagacggactgaaaaaatatatctttttttattttgtctgggcctggagacccgcagcaggagcggaggtgggtgcggggccgggagccggagcaggaccgggaacaggaacaggaacaggaacaggaacaggaacaggagtggggccgggagcaggagcaggagcgggagccgaagtgggggcaggagcggcggcggccgcagcagcaacagggtcgccccagtccgcggcgaggaagagggagctc

SEQ ID NO: 307 - DNA, 494 bp, synthetic construct
  misc_feature 1..494: note = EnhancerLAP2.131
  source 1..494: mol_type = other DNA
gcggcgggtgagtccacgcttccgaacgcgtgaggaaaccgctcgacctgctacctcagctgcgggaggaggaggcggcggcggagaagcatccccggtccgcgctccgcccacccatcacagcagccgcggacgctgcgcgccggagcggtccatctcgccagccagccaaccagccgagccgcccagccgacccgagagccccgagagccagactccctcagccatagaagacaccgggcgggagagacggactgaaaaaatatatctttttttattttgtctgggcctggagacccgcagcaggagcggaggtgggtgcggggccgggagccggagcaggaccgggaacaggaacaggaacaggaacaggaacaggaacaggagtggggccgggagcaggagcaggagcgggagccgaagtgggggcaggagcggcggcggccgcagcagcaacagggtcgccccagtccgcggcgaggaagagggagctc

SEQ ID NO: 308 - DNA, 474 bp, synthetic construct
  misc_feature 1..474: note = EnhancerLAP2.132
  source 1..474: mol_type = other DNA
ctcaaagaggtgagtggtcggctgcgacccctgcctcgggcgctaaggtcaccctgcccagtggtgggagatccccggtccgcgctccgcccacccatcacagcagccgcggacgctgcgcgccggagcggtccatctcgccagccagccaaccagccgagccgcccagccgacccgagagccccgagagccagactccctcagccatagaagacaccgggcgggagagacggactgaaaaaatatatctttttttattttgtctgggcctggagacccgcagcaggagcggaggtgggtgcggggccgggagccggagcaggaccgggaacaggaacaggaacaggaacaggaacaggaacaggagtggggccgggagcaggagcaggagcgggagccgaagtgggggcaggagcggcggcggccgcagcagcaacagggtcgccccagtccgcggcgaggaagagggagctc

SEQ ID NO: 309 - DNA, 484 bp, synthetic construct; identical to SEQ ID NO: 271
  misc_feature 1..484: note = EnhancerLAP2.133
  source 1..484: mol_type = other DNA

SEQ ID NO: 310 - DNA, 504 bp, synthetic construct
  misc_feature 1..504: note = EnhancerLAP2.134
  source 1..504: mol_type = other DNA
ctgtggagggctgcggaggctctagaggggtggggctgggagcgcggaggtggcgaccagaggttcagtactagtttgtggtttagatagtctctttacaatccccggtccgcgctccgcccacccatcacagcagccgcggacgctgcgcgccggagcggtccatctcgccagccagccaaccagccgagccgcccagccgacccgagagccccgagagccagactccctcagccatagaagacaccgggcgggagagacggactgaaaaaatatatctttttttattttgtctgggcctggagacccgcagcaggagcggaggtgggtgcggggccgggagccggagcaggaccgggaacaggaacaggaacaggaacaggaacaggaacaggagtggggccgggagcaggagcaggagcgggagccgaagtgggggcaggagcggcggcggccgcagcagcaacagggtcgccccagtccgcggcgaggaagagggagctc

SEQ ID NO: 311 - DNA, 484 bp, synthetic construct
  misc_feature 1..484: note = EnhancerLAP2.135
  source 1..484: mol_type = other DNA
cgggtgagtggggcgcggcggagcgcgaggcggcccgggcgggaaggcggcgccggcggggacgcaatggcggccacgcgatccccggtccgcgctccgcccacccatcacagcagccgcggacgctgcgcgccggagcggtccatctcgccagccagccaaccagccgagccgcccagccgacccgagagccccgagagccagactccctcagccatagaagacaccgggcgggagagacggactgaaaaaatatatctttttttattttgtctgggcctggagacccgcagcaggagcggaggtgggtgcggggccgggagccggagcaggaccgggaacaggaacaggaacaggaacaggaacaggaacaggagtggggccgggagcaggagcaggagcgggagccgaagtgggggcaggagcggcggcggccgcagcagcaacagggtcgccccagtccgcggcgaggaagagggagctc

SEQ ID NO: 312 - DNA, 474 bp, synthetic construct
  misc_feature 1..474: note = EnhancerLAP2.136
  source 1..474: mol_type = other DNA
gatcccggtgaggaagcggcgggcagatcaggaagtggaaggggcgggacttgagggtgaatggcagcggatccccggtccgcgctccgcccacccatcacagcagccgcggacgctgcgcgccggagcggtccatctcgccagccagccaaccagccgagccgcccagccgacccgagagccccgagagccagactccctcagccatagaagacaccgggcgggagagacggactgaaaaaatatatctttttttattttgtctgggcctggagacccgcagcaggagcggaggtgggtgcggggccgggagccggagcaggaccgggaacaggaacaggaacaggaacaggaacaggaacaggagtggggccgggagcaggagcaggagcgggagccgaagtgggggcaggagcggcggcggccgcagcagcaacagggtcgccccagtccgcggcgaggaagagggagctc

SEQ ID NO: 313 - DNA, 464 bp, synthetic construct; identical to SEQ ID NO: 272
  misc_feature 1..464: note = EnhancerLAP2.137
  source 1..464: mol_type = other DNA

SEQ ID NO: 314 - DNA, 504 bp, synthetic construct
  misc_feature 1..504: note = EnhancerLAP2.138
  source 1..504: mol_type = other DNA
tgcgccaggtgcgccagcggggcgcgcgacggggcgcggggagggcggactcctgatggctttgcctgctcggtgtccggccgcggcctggcctcacccgatccccggtccgcgctccgcccacccatcacagcagccgcggacgctgcgcgccggagcggtccatctcgccagccagccaaccagccgagccgcccagccgacccgagagccccgagagccagactccctcagccatagaagacaccgggcgggagagacggactgaaaaaatatatctttttttattttgtctgggcctggagacccgcagcaggagcggaggtgggtgcggggccgggagccggagcaggaccgggaacaggaacaggaacaggaacaggaacaggaacaggagtggggccgggagcaggagcaggagcgggagccgaagtgggggcaggagcggcggcggccgcagcagcaacagggtcgccccagtccgcggcgaggaagagggagctc

SEQ ID NO: 315 - DNA, 504 bp, synthetic construct
  misc_feature 1..504: note = EnhancerLAP2.139
  source 1..504: mol_type = other DNA
ctcctcctgatccctccccttggttatttaggagattagagttcattaattaaatcctgtgcttagatcgaactgtaacactattccaatcacatttatcatccccggtccgcgctccgcccacccatcacagcagccgcggacgctgcgcgccggagcggtccatctcgccagccagccaaccagccgagccgcccagccgacccgagagccccgagagccagactccctcagccatagaagacaccgggcgggagagacggactgaaaaaatatatctttttttattttgtctgggcctggagacccgcagcaggagcggaggtgggtgcggggccgggagccggagcaggaccgggaacaggaacaggaacaggaacaggaacaggaacaggagtggggccgggagcaggagcaggagcgggagccgaagtgggggcaggagcggcggcggccgcagcagcaacagggtcgccccagtccgcggcgaggaagagggagctc

SEQ ID NO: 316 - DNA, 464 bp, synthetic construct
  misc_feature 1..464: note = EnhancerLAP2.140
  source 1..464: mol_type = other DNA
cccccctccgccccccgcacctgcagcggctcgcgcccagggccccgccccgccagccgcatccccggtccgcgctccgcccacccatcacagcagccgcggacgctgcgcgccggagcggtccatctcgccagccagccaaccagccgagccgcccagccgacccgagagccccgagagccagactccctcagccatagaagacaccgggcgggagagacggactgaaaaaatatatctttttttattttgtctgggcctggagacccgcagcaggagcggaggtgggtgcggggccgggagccggagcaggaccgggaacaggaacaggaacaggaacaggaacaggaacaggagtggggccgggagcaggagcaggagcgggagccgaagtgggggcaggagcggcggcggccgcagcagcaacagggtcgccccagtccgcggcgaggaagagggagctc

SEQ ID NO: 317 - DNA, 504 bp, synthetic construct
  misc_feature 1..504: note = EnhancerLAP2.141
  source 1..504: mol_type = other DNA
ggggcaggcccggggcccggggcggggcggccgtcaggcgcgggctgcggtgctaggccagacggcggggcacgcggtgcggaccggccggggacgcgcaatccccggtccgcgctccgcccacccatcacagcagccgcggacgctgcgcgccggagcggtccatctcgccagccagccaaccagccgagccgcccagccgacccgagagccccgagagccagactccctcagccatagaagacaccgggcgggagagacggactgaaaaaatatatctttttttattttgtctgggcctggagacccgcagcaggagcggaggtgggtgcggggccgggagccggagcaggaccgggaacaggaacaggaacaggaacaggaacaggaacaggagtggggccgggagcaggagcaggagcgggagccgaagtgggggcaggagcggcggcggccgcagcagcaacagggtcgccccagtccgcggcgaggaagagggagctc

SEQ ID NO: 318 - DNA, 474 bp, synthetic construct
  misc_feature 1..474: note = EnhancerLAP22.142
  source 1..474: mol_type = other DNA
gcagttttagggatgttcacagagaagagagacacaagcaaggggcggacacacaaggggcgtggttcttatccccggtccgcgctccgcccacccatcacagcagccgcggacgctgcgcgccggagcggtccatctcgccagccagccaaccagccgagccgcccagccgacccgagagccccgagagccagactccctcagccatagaagacaccgggcgggagagacggactgaaaaaatatatctttttttattttgtctgggcctggagacccgcagcaggagcggaggtgggtgcggggccgggagccggagcaggaccgggaacaggaacaggaacaggaacaggaacaggaacaggagtggggccgggagcaggagcaggagcgggagccgaagtgggggcaggagcggcggcggccgcagcagcaacagggtcgccccagtccgcggcgaggaagagggagctc

SEQ ID NO: 319 - DNA, 484 bp, synthetic construct
  misc_feature 1..484: note = EnhancerLAP2.143
  source 1..484: mol_type = other DNA
tgtccaggtaggaatccacccggctgggcaggctctcggcccacgcggcttcctctccccacgcacctcctccgcgccgtatccccggtccgcgctccgcccacccatcacagcagccgcggacgctgcgcgccggagcggtccatctcgccagccagccaaccagccgagccgcccagccgacccgagagccccgagagccagactccctcagccatagaagacaccgggcgggagagacggactgaaaaaatatatctttttttattttgtctgggcctggagacccgcagcaggagcggaggtgggtgcggggccgggagccggagcaggaccgggaacaggaacaggaacaggaacaggaacaggaacaggagtggggccgggagcaggagcaggagcgggagccgaagtgggggcaggagcggcggcggccgcagcagcaacagggtcgccccagtccgcggcgaggaagagggagctc

SEQ ID NO: 320 - DNA, 494 bp, synthetic construct
  misc_feature 1..494: note = EnhancerLAP2.144 (
  source 1..494: mol_type = other DNA
ctgacagcacctgaagtggaaggaacaaggatgctcaggggacaacatcggggtaacgaccccacctgccctgtcccgcagctcgcctcaatccccggtccgcgctccgcccacccatcacagcagccgcggacgctgcgcgccggagcggtccatctcgccagccagccaaccagccgagccgcccagccgacccgagagccccgagagccagactccctcagccatagaagacaccgggcgggagagacggactgaaaaaatatatctttttttattttgtctgggcctggagacccgcagcaggagcggaggtgggtgcggggccgggagccggagcaggaccgggaacaggaacaggaacaggaacaggaacaggaacaggagtggggccgggagcaggagcaggagcgggagccgaagtgggggcaggagcggcggcggccgcagcagcaacagggtcgccccagtccgcggcgaggaagagggagctc

SEQ ID NO: 321 - DNA, 474 bp, synthetic construct
  misc_feature 1..474: note = EnhancerLAP2.145
  source 1..474: mol_type = other DNA
gcgtctttggtaagctggtcctccctacccaccttccctgccttcccctcctccctgtctggcagcctccatccccggtccgcgctccgcccacccatcacagcagccgcggacgctgcgcgccggagcggtccatctcgccagccagccaaccagccgagccgcccagccgacccgagagccccgagagccagactccctcagccatagaagacaccgggcgggagagacggactgaaaaaatatatctttttttattttgtctgggcctggagacccgcagcaggagcggaggtgggtgcggggccgggagccggagcaggaccgggaacaggaacaggaacaggaacaggaacaggaacaggagtggggccgggagcaggagcaggagcgggagccgaagtgggggcaggagcggcggcggccgcagcagcaacagggtcgccccagtccgcggcgaggaagagggagctc

SEQ ID NO: 322 - DNA, 484 bp, synthetic construct
  misc_feature 1..484: note = EnhancerLAP2.146
  source 1..484: mol_type = other DNA
gtgaggaccacgggggacagggaagagagatgtgtggcgtccgcactgcgggaggctggcatctgccgcaccccccacctatccccggtccgcgctccgcccacccatcacagcagccgcggacgctgcgcgccggagcggtccatctcgccagccagccaaccagccgagccgcccagccgacccgagagccccgagagccagactccctcagccatagaagacaccgggcgggagagacggactgaaaaaatatatctttttttattttgtctgggcctggagacccgcagcaggagcggaggtgggtgcggggccgggagccggagcaggaccgggaacaggaacaggaacaggaacaggaacaggaacaggagtggggccgggagcaggagcaggagcgggagccgaagtgggggcaggagcggcggcggccgcagcagcaacagggtcgccccagtccgcggcgaggaagagggagctc

SEQ ID NO: 323 - DNA, 484 bp, synthetic construct; identical to SEQ ID NO: 274
  misc_feature 1..484: note = EnhancerLAP2.147
  source 1..484: mol_type = other DNA

SEQ ID NO: 324 - DNA, 474 bp, synthetic construct
  misc_feature 1..474: note = EnhancerLAP2.148
  source 1..474: mol_type = other DNA
tggtactaagtatcaaatggaaagggaaagcgagcaggcatatttctgccatggcagaaacagctctcatatccccggtccgcgctccgcccacccatcacagcagccgcggacgctgcgcgccggagcggtccatctcgccagccagccaaccagccgagccgcccagccgacccgagagccccgagagccagactccctcagccatagaagacaccgggcgggagagacggactgaaaaaatatatctttttttattttgtctgggcctggagacccgcagcaggagcggaggtgggtgcggggccgggagccggagcaggaccgggaacaggaacaggaacaggaacaggaacaggaacaggagtggggccgggagcaggagcaggagcgggagccgaagtgggggcaggagcggcggcggccgcagcagcaacagggtcgccccagtccgcggcgaggaagagggagctc

SEQ ID NO: 325 - DNA, 484 bp, synthetic construct; identical to SEQ ID NO: 276
  misc_feature 1..484: note = EnhancerLAP2.149
  source 1..484: mol_type = other DNA

SEQ ID NO: 326 - DNA, 494 bp, synthetic construct; identical to SEQ ID NO: 277
  misc_feature 1..494: note = EnhancerLAP2.150
  source 1..494: mol_type = other DNA

SEQ ID NO: 327 - DNA, 464 bp, synthetic construct
  misc_feature 1..464: note = EnhancerLAP2.151
  source 1..464: mol_type = other DNA
ctcgcgggcgctgtccatggtgcctgcgcgccagccgcacccggccggcgctcgctagctatccccggtccgcgctccgcccacccatcacagcagccgcggacgctgcgcgccggagcggtccatctcgccagccagccaaccagccgagccgcccagccgacccgagagccccgagagccagactccctcagccatagaagacaccgggcgggagagacggactgaaaaaatatatctttttttattttgtctgggcctggagacccgcagcaggagcggaggtgggtgcggggccgggagccggagcaggaccgggaacaggaacaggaacaggaacaggaacaggaacaggagtggggccgggagcaggagcaggagcgggagccgaagtgggggcaggagcggcggcggccgcagcagcaacagggtcgccccagtccgcggcgaggaagagggagctc

SEQ ID NO: 328 - DNA, 464 bp, synthetic construct
  misc_feature 1..464: note = EnhancerLAP2.152
  source 1..464: mol_type = other DNA
caagaagagtgagttgggacaccttgggtggcggctaaggctaggggcggggaactcctaatccccggtccgcgctccgcccacccatcacagcagccgcggacgctgcgcgccggagcggtccatctcgccagccagccaaccagccgagccgcccagccgacccgagagccccgagagccagactccctcagccatagaagacaccgggcgggagagacggactgaaaaaatatatctttttttattttgtctgggcctggagacccgcagcaggagcggaggtgggtgcggggccgggagccggagcaggaccgggaacaggaacaggaacaggaacaggaacaggaacaggagtggggccgggagcaggagcaggagcgggagccgaagtgggggcaggagcggcggcggccgcagcagcaacagggtcgccccagtccgcggcgaggaagagggagctc

SEQ ID NO: 329 - DNA, 494 bp, synthetic construct
  misc_feature 1..494: note = EnhancerLAP2.153
  source 1..494: mol_type = other DNA
gtgagtaaatcctgcgggtaataaacgcggaagtatgaagttgtgccgcaccagcctcagctacccaaattgttggctgcttttgtgattatccccggtccgcgctccgcccacccatcacagcagccgcggacgctgcgcgccggagcggtccatctcgccagccagccaaccagccgagccgcccagccgacccgagagccccgagagccagactccctcagccatagaagacaccgggcgggagagacggactgaaaaaatatatctttttttattttgtctgggcctggagacccgcagcaggagcggaggtgggtgcggggccgggagccggagcaggaccgggaacaggaacaggaacaggaacaggaacaggaacaggagtggggccgggagcaggagcaggagcgggagccgaagtgggggcaggagcggcggcggccgcagcagcaacagggtcgccccagtccgcggcgaggaagagggagctc

SEQ ID NO: 330 - DNA, 494 bp, synthetic construct
  misc_feature 1..494: note = EnhancerLAP2.154
  source 1..494: mol_type = other DNA
ctttcactgtggaatatacacggggttcctcatagctaccatcaccagacccagttccccacaccttggcctgcgcaggcgcccctcactatccccggtccgcgctccgcccacccatcacagcagccgcggacgctgcgcgccggagcggtccatctcgccagccagccaaccagccgagccgcccagccgacccgagagccccgagagccagactccctcagccatagaagacaccgggcgggagagacggactgaaaaaatatatctttttttattttgtctgggcctggagacccgcagcaggagcggaggtgggtgcggggccgggagccggagcaggaccgggaacaggaacaggaacaggaacaggaacaggaacaggagtggggccgggagcaggagcaggagcgggagccgaagtgggggcaggagcggcggcggccgcagcagcaacagggtcgccccagtccgcggcgaggaagagggagctc

SEQ ID NO: 331 - DNA, 494 bp, synthetic construct
  misc_feature 1..494: note = EnhancerLAP2.155
  source 1..494: mol_type = other DNA
attgcatttcctctttccccgcagaacaagatggcggcggtcacgtgggaagggacagccggtacaggttggcgcgtggtccccgccgccatccccggtccgcgctccgcccacccatcacagcagccgcggacgctgcgcgccggagcggtccatctcgccagccagccaaccagccgagccgcccagccgacccgagagccccgagagccagactccctcagccatagaagacaccgggcgggagagacggactgaaaaaatatatctttttttattttgtctgggcctggagacccgcagcaggagcggaggtgggtgcggggccgggagccggagcaggaccgggaacaggaacaggaacaggaacaggaacaggaacaggagtggggccgggagcaggagcaggagcgggagccgaagtgggggcaggagcggcggcggccgcagcagcaacagggtcgccccagtccgcggcgaggaagagggagctc

SEQ ID NO: 332 - DNA, 494 bp, synthetic construct
  misc_feature 1..494: note = EnhancerLAP2.156
  source 1..494: mol_type = other DNA
caaggccatggataggaaaaatccagcagcagatggaaaggcctggatcctagacccaacccgaacccagaggaccccaggggtcccgctatccccggtccgcgctccgcccacccatcacagcagccgcggacgctgcgcgccggagcggtccatctcgccagccagccaaccagccgagccgcccagccgacccgagagccccgagagccagactccctcagccatagaagacaccgggcgggagagacggactgaaaaaatatatctttttttattttgtctgggcctggagacccgcagcaggagcggaggtgggtgcggggccgggagccggagcaggaccgggaacaggaacaggaacaggaacaggaacaggaacaggagtggggccgggagcaggagcaggagcgggagccgaagtgggggcaggagcggcggcggccgcagcagcaacagggtcgccccagtccgcggcgaggaagagggagctc

SEQ ID NO: 333 - DNA, 455 bp, synthetic construct
  misc_feature 1..455: note = EnhancerLAP2.157
  source 1..455: mol_type = other DNA
gcagcagctggcggataattaattagtttaaaagtttgggacagatggcagatccccggtccgcgctccgcccacccatcacagcagccgcggacgctgcgcgccggagcggtccatctcgccagccagccaaccagccgagccgcccagccgacccgagagccccgagagccagactccctcagccatagaagacaccgggcgggagagacggactgaaaaaatatatctttttttattttgtctgggcctggagacccgcagcaggagcggaggtgggtgcggggccgggagccggagcaggaccgggaacaggaacaggaacaggaacaggaacaggaacaggagtggggccgggagcaggagcaggagcgggagccgaagtgggggcaggagcggcggcggccgcagcagcaacagggtcgccccagtccgcggcgaggaagagggagctc

SEQ ID NO: 334 - DNA, 427 bp, synthetic construct
  misc_feature 1..427: note = EnhancerLAP2.158
  source 1..427: mol_type = other DNA
aacatatgtcgcagcagctggcgatccccggtccgcgctccgcccacccatcacagcagccgcggacgctgcgcgccggagcggtccatctcgccagccagccaaccagccgagccgcccagccgacccgagagccccgagagccagactccctcagccatagaagacaccgggcgggagagacggactgaaaaaatatatctttttttattttgtctgggcctggagacccgcagcaggagcggaggtgggtgcggggccgggagccggagcaggaccgggaacaggaacaggaacaggaacaggaacaggaacaggagtggggccgggagcaggagcaggagcgggagccgaagtgggggcaggagcggcggcggccgcagcagcaacagggtcgccccagtccgcggcgaggaagagggagctc

SEQ ID NO: 335 - DNA, 473 bp, synthetic construct
  misc_feature 1..473: note = EnhancerLAP2.159
  source 1..473: mol_type = other DNA
gcagcagctggcgctttgttatgcaaatgtaaacaaggggtaattagctctggagcgttaattaatgtaatccccggtccgcgctccgcccacccatcacagcagccgcggacgctgcgcgccggagcggtccatctcgccagccagccaaccagccgagccgcccagccgacccgagagccccgagagccagactccctcagccatagaagacaccgggcgggagagacggactgaaaaaatatatctttttttattttgtctgggcctggagacccgcagcaggagcggaggtgggtgcggggccgggagccggagcaggaccgggaacaggaacaggaacaggaacaggaacaggaacaggagtggggccgggagcaggagcaggagcgggagccgaagtgggggcaggagcggcggcggccgcagcagcaacagggtcgccccagtccgcggcgaggaagagggagctc

SEQ ID NO: 336 - DNA, 425 bp, synthetic construct
  misc_feature 1..425: note = EnhancerLAP2.160
  source 1..425: mol_type = other DNA
gcagcagctggcggcaattaaatccccggtccgcgctccgcccacccatcacagcagccgcggacgctgcgcgccggagcggtccatctcgccagccagccaaccagccgagccgcccagccgacccgagagccccgagagccagactccctcagccatagaagacaccgggcgggagagacggactgaaaaaatatatctttttttattttgtctgggcctggagacccgcagcaggagcggaggtgggtgcggggccgggagccggagcaggaccgggaacaggaacaggaacaggaacaggaacaggaacaggagtggggccgggagcaggagcaggagcgggagccgaagtgggggcaggagcggcggcggccgcagcagcaacagggtcgccccagtccgcggcgaggaagagggagctc

SEQ ID NO: 337 - DNA, 471 bp, synthetic construct
  misc_feature 1..471: note = EnhancerLAP2.161
  source 1..471: mol_type = other DNA
gcagcagctggcggataattaattagtttaaaagtttgcgaattaattaaaaatctgtttacttaggatccccggtccgcgctccgcccacccatcacagcagccgcggacgctgcgcgccggagcggtccatctcgccagccagccaaccagccgagccgcccagccgacccgagagccccgagagccagactccctcagccatagaagacaccgggcgggagagacggactgaaaaaatatatctttttttattttgtctgggcctggagacccgcagcaggagcggaggtgggtgcggggccgggagccggagcaggaccgggaacaggaacaggaacaggaacaggaacaggaacaggagtggggccgggagcaggagcaggagcgggagccgaagtgggggcaggagcggcggcggccgcagcagcaacagggtcgccccagtccgcggcgaggaagagggagctc

SEQ ID NO: 338 - DNA, 444 bp, synthetic construct
  misc_feature 1..444: note = EnhancerLAP2.162
  source 1..444: mol_type = other DNA
gcagcagctggcggggaaatccccgaattaattaaaaatcatccccggtccgcgctccgcccacccatcacagcagccgcggacgctgcgcgccggagcggtccatctcgccagccagccaaccagccgagccgcccagccgacccgagagccccgagagccagactccctcagccatagaagacaccgggcgggagagacggactgaaaaaatatatctttttttattttgtctgggcctggagacccgcagcaggagcggaggtgggtgcggggccgggagccggagcaggaccgggaacaggaacaggaacaggaacaggaacaggaacaggagtggggccgggagcaggagcaggagcgggagccgaagtgggggcaggagcggcggcggccgcagcagcaacagggtcgccccagtccgcggcgaggaagagggagctc

SEQ ID NO: 339 - DNA, 433 bp, synthetic construct
  misc_feature 1..433: note = EnhancerLAP2.163
  source 1..433: mol_type = other DNA
gcagcagctggcgagggggattagctgccatccccggtccgcgctccgcccacccatcacagcagccgcggacgctgcgcgccggagcggtccatctcgccagccagccaaccagccgagccgcccagccgacccgagagccccgagagccagactccctcagccatagaagacaccgggcgggagagacggactgaaaaaatatatctttttttattttgtctgggcctggagacccgcagcaggagcggaggtgggtgcggggccgggagccggagcaggaccgggaacaggaacaggaacaggaacaggaacaggaacaggagtggggccgggagcaggagcaggagcgggagccgaagtgggggcaggagcggcggcggccgcagcagcaacagggtcgccccagtccgcggcgaggaagagggagctc

SEQ ID NO: 340 - DNA, 505 bp, synthetic construct
  misc_feature 1..505: note = EnhancerLAP2.164
  source 1..505: mol_type = other DNA
gcagcagctggcggataattaattagtttaaaagtttgcgaattaattaaaaatcagtttttaattaatttgtgtttacttaggtgtagggattaattgtcatccccggtccgcgctccgcccacccatcacagcagccgcggacgctgcgcgccggagcggtccatctcgccagccagccaaccagccgagccgcccagccgacccgagagccccgagagccagactccctcagccatagaagacaccgggcgggagagacggactgaaaaaatatatctttttttattttgtctgggcctggagacccgcagcaggagcggaggtgggtgcggggccgggagccggagcaggaccgggaacaggaacaggaacaggaacaggaacaggaacaggagtggggccgggagcaggagcaggagcgggagccgaagtgggggcaggagcggcggcggccgcagcagcaacagggtcgccccagtccgcggcgaggaagagggagctc

SEQ ID NO: 341 - DNA, 444 bp, synthetic construct
  misc_feature 1..444: note = EnhancerLAP2.165
  source 1..444: mol_type = other DNA
gcagcagctggcggggaaatcccagtttttaattaatttgatccccggtccgcgctccgcccacccatcacagcagccgcggacgctgcgcgccggagcggtccatctcgccagccagccaaccagccgagccgcccagccgacccgagagccccgagagccagactccctcagccatagaagacaccgggcgggagagacggactgaaaaaatatatctttttttattttgtctgggcctggagacccgcagcaggagcggaggtgggtgcggggccgggagccggagcaggaccgggaacaggaacaggaacaggaacaggaacaggaacaggagtggggccgggagcaggagcaggagcgggagccgaagtgggggcaggagcggcggcggccgcagcagcaacagggtcgccccagtccgcggcgaggaagagggagctc

SEQ ID NO: 342 - DNA, 429 bp, synthetic construct
  misc_feature 1..429: note = EnhancerLAP2.166
  source 1..429: mol_type = other DNA
aacatatgtcaacaatttcagtgttatccccggtccgcgctccgcccacccatcacagcagccgcggacgctgcgcgccggagcggtccatctcgccagccagccaaccagccgagccgcccagccgacccgagagccccgagagccagactccctcagccatagaagacaccgggcgggagagacggactgaaaaaatatatctttttttattttgtctgggcctggagacccgcagcaggagcggaggtgggtgcggggccgggagccggagcaggaccgggaacaggaacaggaacaggaacaggaacaggaacaggagtggggccgggagcaggagcaggagcgggagccgaagtgggggcaggagcggcggcggccgcagcagcaacagggtcgccccagtccgcggcgaggaagagggagctc

SEQ ID NO: 343 - DNA, 489 bp, synthetic construct
  misc_feature 1..489: note = EnhancerLAP2.167
  source 1..489: mol_type = other DNA
gcagcagctggcggataattaattagtttaaaagtttgaacatatgtcaaattaattaatcatccattagcagaacagatggcagatccccggtccgcgctccgcccacccatcacagcagccgcggacgctgcgcgccggagcggtccatctcgccagccagccaaccagccgagccgcccagccgacccgagagccccgagagccagactccctcagccatagaagacaccgggcgggagagacggactgaaaaaatatatctttttttattttgtctgggcctggagacccgcagcaggagcggaggtgggtgcggggccgggagccggagcaggaccgggaacaggaacaggaacaggaacaggaacaggaacaggagtggggccgggagcaggagcaggagcgggagccgaagtgggggcaggagcggcggcggccgcagcagcaacagggtcgccccagtccgcggcgaggaagagggagctc

SEQ ID NO: 344 - DNA, 453 bp, synthetic construct
  misc_feature 1..453: note = EnhancerLAP2.168
  source 1..453: mol_type = other DNA
aacatatgtcaacaatttcagtgttatccattagcaaaattaattaatcatccccggtccgcgctccgcccacccatcacagcagccgcggacgctgcgcgccggagcggtccatctcgccagccagccaaccagccgagccgcccagccgacccgagagccccgagagccagactccctcagccatagaagacaccgggcgggagagacggactgaaaaaatatatctttttttattttgtctgggcctggagacccgcagcaggagcggaggtgggtgcggggccgggagccggagcaggaccgggaacaggaacaggaacaggaacaggaacaggaacaggagtggggccgggagcaggagcaggagcgggagccgaagtgggggcaggagcggcggcggccgcagcagcaacagggtcgccccagtccgcggcgaggaagagggagctc

SEQ ID NO: 345 - DNA, 260 bp, synthetic construct
  misc_feature 1..260: note = EnhancerLAP2.169
  source 1..260: mol_type = other DNA
tcagccaatcagcgaggccgcggacgctatatatctttttttattttgtctgggcctggagacccgcagcaggagcggaggtgggtgcggggccgggagccggagcaggaccgggaacaggaacaggaacaggaacaggaacaggaacaggagtggggccgggagcaggagcaggagcgggagccgaagtgggggcaggagcggcggcggccgcagcagcaacagggtcgccccagtccgcggcgaggaagagggagctc

SEQ ID NO: 346 - DNA, 258 bp, synthetic construct
  misc_feature 1..258: note = EnhancerLAP2.170
  source 1..258: mol_type = other DNA
ccgcgaggaggcaggccgcggacgctatatatctttttttattttgtctgggcctggagacccgcagcaggagcggaggtgggtgcggggccgggagccggagcaggaccgggaacaggaacaggaacaggaacaggaacaggaacaggagtggggccgggagcaggagcaggagcgggagccgaagtgggggcaggagcggcggcggccgcagcagcaacagggtcgccccagtccgcggcgaggaagagggagctc

SEQ ID NO: 347 - DNA, 278 bp, synthetic construct
  misc_feature 1..278: note = EnhancerLAP2.171
  source 1..278: mol_type = other DNA
tggtgccctctggtggtcttctagaggaattgcagccgcggacgctatatatctttttttattttgtctgggcctggagacccgcagcaggagcggaggtgggtgcggggccgggagccggagcaggaccgggaacaggaacaggaacaggaacaggaacaggaacaggagtggggccgggagcaggagcaggagcgggagccgaagtgggggcaggagcggcggcggccgcagcagcaacagggtcgccccagtccgcggcgaggaagagggagctc

SEQ ID NO: 348 - DNA, 363 bp, synthetic construct
  misc_feature 1..363: note = EnhancerLAP2.172
  source 1..363: mol_type = other DNA
tcagccaatcagcgagcacccatcacagcagccgcggacgctgcgcgccggagcggtccatctcgccagccagccaaccagccgagccgcccagccgacccgagagccccgagagccagactccctcagccatagaagacaccgggcgggagagacggactgaaaaaatatatctttttttattttgtctgggcctggagacccgcagcaggagcggaggtgggtgcggggccgggagccggagcaggaccgggaacaggaacaggaacaggaacaggaacaggaacaggagtggggccgggagcaggagcaggagcgggagccgaagtgggggcaggagcggcggcggccgcagcagcaaca

SEQ ID NO: 349 - DNA, 253 bp, synthetic construct
  source 1..253: mol_type = other DNA
tggtgccctctggtggtcttctagaggaattgcaatccccggtccgcgctccgcccacccatcacagcagccgcggacgctgcgcgccggagcggtccatctcgccagccagccaaccagccgagccgcccagccgacccgagagccccgagagccagactccctcagccatagaagacaccgggcggcgggagagacggactgaaaaaatatatctttttttattttgtctgggcctggagacccgcagcag

SEQ ID NO: 350 - DNA, 34 bp, synthetic construct
  source 1..34: mol_type = other DNA
tggtgccctctggtggtcttctagaggaattgca

SEQ ID NO: 351 - DNA, 902 bp, synthetic construct
  source 1..902: mol_type = other DNA
gggggagtcgggggggagtcgggggggagtcggggggtcccagttcggcgcggtctccacagcgcacaaagtccggctgcagtaaatttactgcgggtgcagttaccctggcggccacaccggcccgatggcgctgcagtaactctactgcagctacagtttactacagttgcagtaccgcgcgccgccgccaaatactacagtagatttcctgtggccgccgcgtactgcagtttaccgcggctgcagtaaactgcagtatcgcgcggtaaattgcagtctggcagccgcgcgttactgcaattagcggtggctcccgacactctggccaattggtgctaatgggccgtgatggtccatgtgggggtgatgtaaccgccgggcccggttgggcactcagatggtggccgggcgccaggccaaagtgctgtctgagtgccactttatgactttgtttttctcaaacaacatcaattatggatgcacatcgtgtatataatccccggtccgcgctccgcccacccatcacagcagccgcggacgctgcgcgccggagcggtccatctcgccagccagccaaccagccgagccgcccagccgacccgagagccccgagagccagactccctcagccatagaagacaccgggcgggagagacggactgaaaaaatatatctttttttattttgtctgggcctggagacccgcagcaggagcggaggtgggtgcggggccgggagccggagcaggaccgggaacaggaacaggaacaggaacaggaacaggaacaggagtggggccgggagcaggagcaggagcgggagccgaagtgggggcaggagcggcggcggccgcagcagcaacagggtcgccccagtccgcggcgaggaagagggagctc